(19) United States
(12) Patent Application Publication
(10) Pub. No.: US 2006/0292150 A1
(43) Pub. Date: Dec. 28, 2006

(54) Methods of using wisp antagonists

(76) Inventor: Luc Desnoyers, San Francisco, CA (US)

Correspondence Address: GENENTECH, INC., 1 DNA WAY, SOUTH SAN FRANCISCO, CA, 94080, US

(21) Appl. No.: 10/570,826

(22) PCT Filed: Sep. 9, 2004
(86) PCT No.: PCT/US04/29510

§ 371 Date: Mar. 6, 2006

Related U.S. Application Data
(60) Provisional application No. 60/502,013, filed on Sep. 11, 2003.

(51) Int. Cl. A61K 39/395; A61K 38/54

(52) U.S. Cl. 424/146.1; 514/12

(57) ABSTRACT

Methods and compositions for use in blocking or inhibiting the activity(s) of WISP-1 polypeptide on chondrocytes are provided. WISP-1 antagonists include anti-WISP-1 antibodies, WISP-1 immunoadhesins and WISP-1 variants (and fusion proteins thereof) which inhibit or neutralize the effects of WISP-1 on mammalian chondrocytes.

### RELATED APPLICATIONS

This application is a non-provisional application claiming priority under Section 119(e) to provisional application No. 60/502,013 filed Sep. 11, 2003, the contents of which are incorporated herein by reference.

### FIELD OF THE INVENTION

The present invention relates generally to methods of using WISP antagonists in the treatment of chondrocyte and cartilage-related disorders.

### BACKGROUND OF THE INVENTION

Connective tissue growth factor (CTGF) is a growth factor induced in fibroblasts by many factors, including TGF-β, and is essential for the ability of TGF-β to induce anchorage-independent growth (AIG), a property of transformed cells. CTGF was discovered in an attempt to identify the type of platelet-derived growth factor (PDGF) dimers present in the growth media of cultured endothelial cells. See U.S. Pat. No. 5,408,040. CTGF is also mitogenic and chemotactic for cells, and hence growth factors in this family are believed to play a role in the normal development, growth, and repair of human tissue.

Proteins related to CTGF, including the chicken ortholog for Cyr61, CEF10, human, mouse, and Xenopus laevis CTGF, and human, chicken, and Xenopus laevis Nov have been isolated, cloned, sequenced, and characterized as belonging to the CCN gene family. Oemar and Luescher, Arterioscler. Thromb. Vasc. Biol., 17: 1483-1489 (1997). Cyr61 promotes angiogenesis, tumor growth, and vascularization. Babic et al., Proc. Natl. Acad. Sci. USA, 95: 6355-6360 (1998). The nov gene is expressed in the kidney at the embryonic stage, and alterations of nov expression, relative to the normal kidney, have been detected in both avian nephroblastomas and human Wilms' tumors. Martinerie et al., Oncogene, 9: 2729-2732 (1994). Wt1 downregulates nov expression, which downregulation might represent a key element in normal and tumoral nephrogenesis. Martinerie et al., Oncogene, 12: 1479-1492 (1996). The different members of the CCN family interact with various soluble or matrix associated macromolecules in particular sulfated glycoconjugates (Holt et al., J. Biol. Chem., 265:2852-2855 (1990)). This interaction was used to purify Cyr61 and CTGF by affinity chromatography on heparin-agarose (Frazier et al., J. Invest. Dermatol., 107:404-411 (1996); Kireeva et al., Mol. Cell. Biol., 16:1326-1334 (1996)). Cyr61 is secreted and associated with both the extracellular matrix and the cell surface due to its affinity for heparan sulfate (Yang et al., Cell. Growth Diff., 2:351-357 (1991)).

ELM1 was identified in low metastatic mouse cells. Hashimoto et al., J. Exp. Med., 187: 289-296 (1998). The elm1gene, the mouse orthologue of WISP-1 disclosed below, is another member of the CTGF, Cyr61/Cef10, and neuroblastoma overexpressed-gene family and suppresses in vivo tumor growth and metastasis of K-1735 murine melanoma cells. Another recent article on rCop-1, the rat orthologue of WISP-2 described below describes the loss of expression of this gene after cell transformation. Zhang et al., Mol. Cell. Biol., 18:6131-6141 (1998).

CCN family members (with the exception of nov) are immediate early growth-responsive genes that are thought to regulate cell proliferation, differentiation, embryogenesis, and wound healing. Sequence homology among members of the CCN gene family is somewhat high; however, functions of these proteins in vitro range from growth stimulatory (i.e., human CTGF) to growth inhibitory (i.e., chicken Nov and also possibly hCTGF). Further, some molecules homologous to CTGF are indicated to be useful in the prevention of desmoplasia, the formation of highly cellular, excessive connective tissue stroma associated with some cancers, and fibrotic lesions associated with various skin disorders such as scleroderma, keloid, eosinophilic fasciitis, nodular fasciitis, and Dupuytren's contracture. Moreover, CTGF expression has recently been demonstrated in the fibrous stroma of mammary tumors, suggesting cancer stroma formation involves the induction of similar fibroproliferative growth factors as wound repair. Human CTGF is also expressed at very high levels in advanced atherosclerotic lesions, but not in normal arteries, suggesting it may play a role in atherosclerosis. Oemar and Luescher, supra.

Wnts are encoded by a large gene family whose members have been found in round worms, insects, cartilaginous fish, and vertebrates. Holland et al., Dev. Suppl., 125-133 (1994). Wnts are thought to function in a variety of developmental and physiological processes since many diverse species have multiple conserved Wnt genes. McMahon, Trends Genet., 8: 236-242 (1992); Nusse and Varmus, Cell, 69: 1073-1087 (1992). Wnt genes encode secreted glycoproteins that are thought to function as paracrine or autocrine signals active in several primitive cell types. McMahon, supra (1992); Nusse and Varmus, supra (1992). The Wnt growth factor family includes more than ten genes identified in the mouse (Wnt-1, -2, -3A, -3B, -4, -5A, -5B, -6, -7A, -7B, -8A, -8B, -10B, -11, -12, and -13) (see, e.g., Gavin et al., Genes Dev., 4: 2319-2332 (1990); Lee et al., Proc. Natl. Acad. Sci. USA, 92: 2268-2272 (1995); Christiansen et al., Mech. Dev., 51: 341-350 (1995)) and at least nine genes identified in the human (Wnt-1, -2, -3, -5A, -7A, -7B, -8B, -10B, and -11) by CDNA cloning. See, e.g., Vant Veer et al., Mol. Cell. Biol., 4: 2532-2534 (1984).

The Wnt-1 proto-oncogene (int-1) was originally identified from mammary tumors induced by mouse mammary tumor virus (MMTV) due to an insertion of viral DNA sequence. Nusse and Varmus, Cell, 31: 99-109 (1982). In adult mice, the expression level of Wnt-1 mRNA is detected only in the testis during later stages of sperm development. Wnt-1 protein is about 42 kDa and contains an amino-terminal hydrophobic region, which may function as a signal sequence for secretion (Nusse and Varmus, supra, 1992). The expression of Wnt-2/irp is detected in mouse fetal and adult tissues and its distribution does not overlap with the expression pattern for Wnt-1. Wnt-3 is associated with mouse mammary tumorigenesis. The expression of Wnt-3 in mouse embryos is detected in the neural tubes and in the limb buds. Wnt-5a transcripts are detected in the developing fore- and hind limbs at 9.5 through 14.5 days and highest levels are concentrated in apical ectoderm at the distal tip of limbs. Nusse and Varmus, supra (1992). Recently, a Wnt growth factor, termed Wnt-x, was described (WO95/17416) along with the detection of Wnt-x expression in bone tissues and in bone-derived cells. Also described was the role of Wnt-x in the maintenance of mature osteoblasts and the use of the Wnt-x growth factor as a therapeutic agent or in the development of other therapeutic agents to treat bone-related diseases. It has been described that the Wnt pathway may affect growth, patterning and morphogenesis of skeletal elements by modulating chondrocytes and osteoblast differentiation. Gong et al., Cell, 107: 513-523 (2001); Hartmann et al., Development, 127: 3141-3159 (2000); Hartmann and Tabin, Cell, 104: 341-351 (2001); Rudnicki and Brown, Dev Biol, 185:104-118 (1997).

Wnts may play a role in local cell signaling. Biochemical studies have shown that much of the secreted Wnt protein can be found associated with the cell surface or extracellular matrix rather than freely diffusible in the medium. Papkoff and Schryver, Mol. Cell. Biol., 10: 2723-2730 (1990); Bradley and Brown, EMBO J., 9: 1569-1575 (1990).

Studies of mutations in Wnt genes have indicated a role for Wnts in growth control and tissue patterning. In Drosophila, wingless (wg) encodes a Wnt-related gene (Rijsewik et al., Cell, 50: 649-657 (1987)) and wg mutations alter the pattern of embryonic ectoderm, neurogenesis, and imaginal disc outgrowth. Morata and Lawerence, Dev. Biol., 56: 227-240 (1977); Baker, Dev. Biol., 125: 96-108 (1988); Klingensmith and Nusse, Dev. Biol., 166: 396-414 (1994). In Caenorhabditis elegans, lin-44 encodes a Wnt homolog which is required for asymmetric cell divisions. Herman and Horvitz, Development, 120: 1035-1047 (1994). Knock-out mutations in mice have shown Wnts to be essential for brain development (McMahon and Bradley, Cell, 62: 1073-1085 (1990); Thomas and Cappechi, Nature, 346: 847-850 (1990)), and the outgrowth of embryonic primordia for kidney (Stark et al., Nature, 372: 679-683 (1994)), tail bud (Takada et al., Genes Dev., 8: 174-189 (1994)), and limb bud. Parr and McMahon, Nature, 374: 350-353 (1995). Overexpression of Wnts in the mammary gland can result in mammary hyperplasia (McMahon, supra (1992); Nusse and Varmus, supra (1992)), and precocious alveolar development. Bradbury et al., Dev. Biol., 170: 553-563 (1995).

Wnt-5a and Wnt-5b are expressed in the posterior and lateral mesoderm and the extraembryonic mesoderm of the day 7-8 murine embryo. Gavin et al., supra (1990). These embryonic domains contribute to the AGM region and yolk sac tissues from which multipotent hematopoietic precursors and HSCs are derived. Dzierzak and Medvinsky, Trends Genet., 11: 359-366 (1995); Zon et al., in Gluckman and Coulombel, ed., Colloque, INSERM, 235: 17-22 (1995), presented at the Joint International Workshop on Foetal and Neonatal Hematopoiesis and Mechanism of Bone Marrow Failure, Paris France, Apr. 3-6, 1995; Kanatsu and Nishikawa, Development, 122 : 823-830 (1996). Wnt-5a, Wnt-10b, and other Wnts have been detected in limb buds, indicating possible roles in the development and patterning of the early bone microenvironment as shown for Wnt-7b. Gavin et al., supra (1990); Christiansen et al., Mech. Devel., 51: 341-350 (1995); Parr and McMahon, supra (1995).

The Wnt/Wg signal transduction pathway plays an important role in the biological development of the organism and has been implicated in several human cancers. This pathway also includes the tumor suppressor gene, APC. Mutations in the APC gene are associated with the development of sporadic and inherited forms of human colorectal cancer. The Wnt/Wg signal leads to the accumulation of beta-catenin/Armadillo in the cell, resulting in the formation of a bipartite transcription complex consisting of beta-catenin and a member of the lymphoid enhancer binding factor/T cell factor (LEF/TCF)HMG box transcription factor family. This complex translocates to the nucleus where it can activate expression of genes downstream of the Wnt/Wg signal, such as the engrailed and Ultr-abithorax genes in Drosophila.

For a review on Wnt, see Cadigan and Nusse, Genes & Dev., 11: 3286-3305 (1997).

Pennica et al., Proc. Natl. Acad. Sci., 95:14717-14722 (1998) describe the cloning and characterization of two genes, WISP-1 and WISP-2, and a third related gene, WISP-3. Pennica et al. report that these WISP genes may be downstream of Wnt-1 signaling and that aberrant levels of WISP expression in colon cancer may play a role in colon tumorigenesis. WISP-1 has recently been identified as a β-catenin-regulated gene and the characterization of its oncogenic activity demonstrated that WISP-1 might contribute to β-catenin-mediated tumorigenesis (Xu et al., Gene & Develop., 14:585-595 (2000)). WISP-1 overexpression in normal rat kidney cells (NRK-49F) induced morphological transformation, accelerated cell growth and enhanced saturation density. In addition, these cells readily form tumors when injected into nude mice suggesting that WISP-1 may play sorne role in tumorigenesis (Xu et al., supra 2000).

Hurvitz et al., Nature Genetics, 23:94-97 (1999) describe a study involving WISP3 in which nine different mutations of WISP3 in unrelated individuals were found to be associated with the autosomal recessive skeletal disorder, progressive pseudorheumatoid dysplasia (PPD). WISP3 expression by RT-PCR was observed by Hurvitz et al. in human synoviocytes, articular cartilage chondrocytes, and bone-marrow-derived mesenchymal progenitor cells.

PCT application WO98/21236 published May 22, 1998 discloses a human connective tissue growth factor gene-3 (CTGF-3) encoding a 26 kDa member of the growth factor superfamily. WO98/21236 discloses that the CTGF-3 amino acid sequence was deduced from a human osteoblast cDNA clone, and that CTGF-3 was expressed in multiple tissues like ovary, testis, heart, lung, skeletal muscle, adrenal medulla, adrenal cortex, thymus, prostate, small intestine and colon.

Several investigators have documented changes in the proteoglycan composition in neoplasms. Especially, a marked production of chondroitin sulfate proteoglycan is a well-recognized phenomenon in a variety of malignant tumors. In addition, the expression of decorin, a dermatan sulfate containing proteoglycan, has been shown to be well correlated with malignancy in human carcinoma (Adany et al., J. Biol. Chem., 265:11389-11396 (1990); Hunzlemann et al., J. Invest. Dermatol., 104:509-513 (1995)). It was demonstrated that decorin suppresses the growth of several carcinomas (Santra 1997). Although the function of decorin in tumorigenic development is not fully understood, it was proposed that the decorin expression in the peritumorous stroma may reflect a regional response of the host connective tissue cells to the invading neoplastic cells (Ständer et al., Gene Therapy, 5:1187-1194 (1999)).

For a recent review of various members of the connective tissue growth factor/cysteine-rich 61/nephroblastoma overexpressed (CNN) family, and their respective properties and activities, see Brigstock, Endocrine Reviews, 20:189-206 (1999).

Degenerative cartilagenous disorders broadly describe a collection of diseases characterized by degeneration or metabolic abnormalities of the connective tissues which can be manifested by pain, stiffness and limitation of motion of the affected body parts. The origin of these disorders can be, for example, pathological or as a result of trauma or injury.

Osteoarthritis (OA), also known as osteoarthrosis or degenerative joint disease, is typically the result of a series of localized degenerative processes that affect the articular structure and result in pain and diminished function. OA is often accompanied by a local inflammatory component that may accelerate joint destruction. OA is characterized by disruption of the smooth articulating surface of cartilage, with early loss of proteoglycans (PG) and collagens, followed by formation of clefts and fibrillation, and ultimately by full-thickness loss of cartilage. OA symptoms include local pain at the affected joints, especially after use. With disease progression, symptoms may progress to a continuous aching sensation, local discomfort and cosmetic alterations such as deformity of the affected joint.

In contrast to the localized nature of OA, rheumatoid arthritis (RA) is a systemic, inflammatory disease which likely begins in the synovium, the tissues surrounding the joint space. RA is a chronic autoimmune disorder characterized by symmetrical synovitis of the joint and typically affects small and large diarthrodial joints, leading to their progressive destruction. As the disease progresses, the symptoms of RA may also include fever, weight loss, thinning of the skin, multiorgan involvement, scleritis, corneal ulcers, formation of subcutaneous or subperiosteal nodules and premature death. While the cause(s) or origins of RA and OA are distinctly different, the cytokines and enzymes involved in cartilage destruction appear to be similar.

Peptide growth factors are believed to be important regulators of cartilage growth and cartilage cell (chondrocyte) behavior (i.e., differentiation, migration, division, and matrix synthesis or breakdown) F. S. Chen et al., Am J. Orthop. 26: 396-406 (1997). Growth factors that have been previously proposed to stimulate cartilage repair include insulin-like growth factor (IGF-1), Osborn, J. Orthop. Res. 7: 35-42 (1989); Florini & Roberts, J. Gerontol. 35: 23-30 (1980); basic fibroblast growth factor (bFGF), Toolan et al., J. Biomec. Mat. Res. 41: 244-50 (1998); Sah et al., Arch. Biochem. Biophys. 308: 137-47 (1994); bone morphogenetic protein (BMP), Sato & Urist, Clin. Orthop. Relat. Res. 183: 180-87 (1984); Chin et al., Arthritis Rheum. 34: 314-24 (1991) and transforming growth factor beta (TGF-beta), Hill & Logan, Prog. Growth Fac. Res. 4: 45-68 (1992); Guerne et al., J. Cell Physiol. 158: 476-84 (1994); Van der Kraan et al., Ann. Rheum. Dis. 51: 643-47 (1992).

Insulin-like growth factor (IGF-1) stimulates both matrix synthesis and cell proliferation in culture, K. Osborn. J. Orthop. Res. 7: 35-42 (1989), and insufficiency of IGF-1 may have an etiologic role in the development of osteoarthritis. R. D. Coutts, et al., Instructional Course Lect. 47: 487-94, Amer. Acad. Orthop. Surg. Rosemont, Ill. (1997). Some studies indicate that serum IGF-1 concentrations are lower in osteoaxthritic patients than control groups, while other studies have found no difference. Nevertheless, both serum IGF-1 levels and chondrocyte responsiveness to IGF-1 decrease with age. J. R. Florini & S. B. Roberts, J. Gerontol. 35: 23-30 (1980). Thus, both the decreased availability of IGF-1 as well as diminished chondrocyte responsiveness to IGF-1 may contribute to cartilage homeostasis and lead to degeneration with advancing age.

IGF-1 has been proposed for the treatment of prevention of osteoarthritis. Intra-articular administration of IGF-1 in combination with sodium pentosan polysulfate (a chondrocyte catabolic activity inhibitor) caused improved histological appearance, and near-normal levels of degradative enzymes (neutral metalloproteinases and collagenase), tissue inhibitors of metalloproteinase and matrix collagen. R. A. Rogachefsky, et al., Ann. NY Acad. Sci. 732: 889-95 (1994). The use of IGF-1 either alone or as an adjuvant with other growth factors to stimulate cartilage regeneration has been described in WO 91/19510, Wo 92/13565, U.S. Pat. No. 5,444,047, and EP 434,652,

Bone morphogenetic proteins (BMPs) are members of the large transforming growth factor beta (TGF-β) family of growth factors. In vitro and in vivo studies have shown that BMP induces the differentiation of mesenchymal cells into chondrocytes. K. Sato & M. Urist, Clin. Orthop. Relat. Res. 183: 180-87 (1984). Furthermore, skeletal growth factor and cartilage-derived growth factors have synergistic effects with BMP, as the combination of these growth factors with BMP and growth hormone initiates mesenchymal cell differentiation. Subsequent proliferation of the differentiated cells are stimulated by other factors. D. J. Hill & A Logan, Prog. Growth Fac. Res. 4: 45-68 (1992).

Transforming growth factor beta (TGF-β) is produced by osteoblasts, chondrocytes, platelets, activated lymphocytes, and other cells. R. D. Coutts et al., supra. TGF-β can have both stimulatory and inhibitory properties on matrix synthesis and cell proliferation depending on the target cell, dosage, and cell culture conditions. P. Guerne et al., J. Cell Physiol. 158: 476-84 (1994); H. Van Beuningen et al., Ann. Rheum. Dis. 52: 185-91 (1993); P. Van der Kraan et al., Ann. Rheum. Dis. 51: 643-47 (1992). Furthermore, as with IGF-1, TGF-β responsiveness is decreased with age. P. Guerne et al., J. Cell Physiol. 158: 476-84 (1994). However, TGF-β is a more potent stimulator of chondrocyte proliferation than other growth factors, including platelet-derived growth factor (PDGF), bFGF, and IGF-1 (Guerne et al., supra), and can stimulate proteoglycan production by chondrocytes. TGF-β also down-regulates the effects of cytokines which stimulate chondrocyte catabolism Van der Kraan et al., supra. In vivo, TGF-β induces proliferation and differentiation of mesenchymal cells into chondrocytes and enhances repair of partial-thickness defects in rabbit articular cartilage. E. B. Hunziker & L. Rosenberg, Trans. Orthopaed. Res. Soc. 19: 236 (1994).

While some investigators have focused on the use of certain growth factors to repair cartilage or chondrocyte tissue, others have looked at inhibiting the activity of molecules which induce cartilage destruction and/or inhibit matrix synthesis. One such molecule is the cytokine IL-1alpha, which has detrimental effects on several tissues within the joint, including generation of synovial inflammation and up-regulation matrix metalloproteinases and prostaglandin expression. V. Baragi, et al., J. Clin. Invest. 96: 2454-60 (1995); V. M. Baragi et al., Osteoarthritis Cartilage 5: 275-82 (1997); C. H. Evans et al., J. Keukoc. Biol. 64: 55-61 (1998); C. H Evans and P. D. Robbins, J. Rheumatol. 24: 2061-63 (1997) ; R. Kang et al., Biochem. Soc. Trans. 25: 533-37 (1997); R. Kang et al., Osteoarthritis Cartilage 5: 139-43 (1997). One means of antagonizing IL-1alpha is through treatment with soluble IL-1 receptor antagonist (IL-1ra), a naturally occurring protein that prevents IL-1 from binding to its receptor, thereby inhibiting both direct and indirect effects of IL-1 on cartilage. In mammals only one protease, named interleukin 1beta-convertase (ICE), can specifically generate mature, active IL-1alpha. Inhibition of ICE has been shown to block IL-1alpha production and may slow arthritic degeneration (reviewed in Martel-Pelletier J. et al. Front. Biosci. 4: d694-703). The soluble IL-1 receptor antagonist (IL-1ra), a naturally occurring protein that can inhibit the effects of IL-1 by preventing IL-1 from interacting with chondrocytes, has also been shown to be effective in animal models of arthritis and is currently being tested in humans for its ability to prevent incidence or progression of arthritis. Other cytokines, such as IL-1beta, tumor necrosis factor-alpha, interferon gamma, IL-6, and IL-8 have been linked to increased activation of synovial fibroblast-like cells, chondrocytes and/or macrophages. The inhibition of these cytokines may be of therapeutic benefit in preventing inflammation and cartilage destruction. Molecules which inhibit TNF-alpha activity have been shown to have beneficial effects on the joints of patients with rheumatoid arthritis.

Cartilage matrix degradation is believed to be due to cleavage of matrix molecules (proteoglycans and collagens) by proteases (reviewed in Woessner J F Jr., “Proteases of the extracellular matrix”, in Mow, V., Ratcliffe, A. (eds): Structure and Function of Articular Cartilage. Boca Raton, Fla., CRC Press, 1994 and Smith R. L., Front. In Biosci. 4:d704-712. While the key enzymes involved in matrix breakdown have not yet been clearly identified, matrix metalloproteinases (MMPs) and “aggrecanases” appear to play key roles in joint destruction. In addition, members of the serine and cysteine family of proteinases (for example, the cathepsins and urokinase or tissue plasminogen activator (uPA and tPA)) may also be involved. Plasmin, urokinase plasminogen activator (uPA) and tissue plasminogen activator (tPA) may play an important role in the activation pathway of the metalloproteinases. Evidence connects the closely related group of cathepsin B, L and S to matrix breakdown, and these cathepsins are scmewhat increased in OA. Many cytokines, including IL-1, TNF-alpha and LIF induce MMP expression in chondrocytes. Induction of MMPs can be antagonized by TGF-β and IL-4 and potentiated, at least in rabbits, by FGF and PDGF. As shown by animal studies, inhibitors of these proteases (MMPs and aggrecanases) may at least partially protect joint tissue from damage in vivo.

Nitric oxide (NO) may also play a substantial role in the destruction of cartilage. Ashok et al., Curr. Opin. Rheum. 10: 263-268 (1998). Unlike normal cartilage which does not produce NO unless stimulated with cytokines such as IL-1, cartilage obtained from osteoarthritic joints produces large amounts of nitric oxide for over 3 days in culture despite the absence of added stimuli. Moreover, inhibition of NO production has been shown to prevent IL-1 mediated cartilage destruction and chondrocyte death as well as progression of osteoarthritis in animal models.

### SUMMARY OF THE INVENTION

Applicants have found that certain WISP polypeptides may block or inhibit chondrocyte differentiation. Accordingly, it is presently believed that molecules which antagonize such activity (e.g., WISP antagonists) can be useful for the treatment of disorders, for instance, affecting cartilage repair, including osteoarthritis.

In one embodiment, the present invention concerns a method for the treatment of damaged cartilage comprising contacting said affected joint tissue with an effective amount of WISP antagonist. WISP antagonists contemplated for use in the invention include but are not limited to WISP-1 antibodies and WISP-1 polypeptides consisting of select domains of WISP-1, described further below. Optionally, the tissue is cartilage, and the amount of WISP antagonist employed is a therapeutically effective amount. In a preferred embodiment, the disorder is osteoarthritis. The methods may be conducted in vivo, such as by administering the therapeutically effective amount of WISP antagonist to the mammal, or ex vivo, by contacting said cartilage tissue with an effective amount of WISP antagonist in culture and then transplanting the treated cartilage tissue into the mammal. In addition, the methods may be conducted by employing WISP antagonist alone as a therapeutic agent, or in combination with an effective amount of another agent or other therapeutic technique. For example, the WISP antagonist may be employed in combination with any standard surgical technique. The WISP antagonist may be administered prior, after and/or simultaneous to the standard surgical technique.

In a further embodiment, the present invention concerns a method for the treatment of cartilage damaged by injury or preventing the initial or continued damage comprising contacting said cartilage tissue with an effective amount of WISP antagonist. More specifically, the injury treated is microdamage or blunt trauma, a chondral fracture, an osteochondral fracture, or damage to tendons, menisci, or ligaments. In a specific aspect, the injury can be the result of excessive mechanical stress or other biomechanical instability resulting from a sports injury or obesity.

In another embodiment, the invention concerns a method of stimulating differentiation of chondrocyte precursor cells by contacting the chondrocyte precursor cells with an effective amount of WISP antagonist.

In another embodiment, the present invention concerns a kit or article of manufacture, comprising WISP antagonist and a carrier, excipient and/or stabilizer (e.g. a buffer) in suitable packaging. The kit or article preferably contains instructions for using WISP antagonist to treat cartilage or to prevent initial or continued damage to cartilage tissue as a result of a disorder. Alternatively, the kit may contain instructions for using WISP antagonist to treat a cartilage disorder.

More particular embodiments of the present invention include methods of treating mammalian cartilage cells or tissue, comprising contacting mammalian cartilage cells or tissue damaged from a degenerative cartilagenous disorder (or damaged from an injury) with an effective amount of WISP antagonist.

Various embodiments of the invention are illustrated more particularly by the following claims:
1. A method for treating damaged cartilage tissue comprising contacting said cartilage tissue with an effective amount of WISP antagonist.
2. The method of claim 1 wherein said WISP antagonist is selected from the group consisting of a WISP-1 antibody, WISP-1 immunoadhesin, WISP-1 polypeptide, and WISP-1 variant.
3. The method of claim 2 wherein said WISP-1 polypeptide consists of Domain 1 amino acids 24 to 117 of human WISP-1 (SEQ ID NO:1).
4. The method of claim 2 wherein said WISP antagonist is a WISP-1 monoclonal antibody.
5. The method of claim 4 wherein said WISP-1 monoclonal antibody is a human antibody, chimeric antibody or humanized antibody.
6. The method of claim 1 wherein said cartilage tissue is articular cartilage tissue.
7. The method of claim 1 wherein said effective amount of WISP antagonist is contacted with the damaged cartilage tissue in vivo in a mammal.
8. The method of claim 1 wherein said effective amount of WISP antagonist is contacted with the damaged cartilage tissue in vitro and subsequently transplanted into a mammal.
9. A method of stimulating differentiation of chondrocyte precursor cells, comprising contacting mammalian chondrocyte precursor cells with an effective amount of WISP antagonist.
10. The method of claim 9 wherein said WISP antagonist is selected from the group consisting of a WISP-1 antibody, WISP-1 immunoadhesin, WISP-1 polypeptide, and WISP-1 variant.
11. The method of claim 10 wherein said WISP-1 polypeptide consists of Domain 1 amino acids 24 to 117 of human WISP-1 (SEQ ID NO:1).
12. The method of claim 10 wherein said WISP antagonist is a WISP-1 monoclonal antibody.
13. The method of claim 12 wherein said WISP-1 monoclonal antibody is a human antibody, chimeric antibody or humanized antibody.
14. The method of claim 9 wherein said effective amount of WISP antagonist is contacted with the chondrocyte precursor cells in vivo in a mammal.
15. The method of claim 9 wherein said effective amount of WISP antagonist is contacted with the chondrocyte precursor cells in vitro and subsequently transplanted into a mammal.
16. A method of treating a cartilagenous disorder in a mammal, comprising administering an effective amount of WISP antagonist to said mammal.
17. The method of claim 16 wherein said WISP antagonist is selected from the group consisting of a WISP-1 antibody, WISP-1 immunoadhesin, WISP-1 polypeptide, and WISP-1 variant.
18. The method of claim 17 wherein said WISP-1 polypeptide consists of Domain 1 amino acids 24 to 117 of human WISP-1 (SEQ ID NO:1).
19. The method of claim 17 wherein said WISP antagonist is a WISP-1 monoclonal antibody.
20. The method of claim 19 wherein said WISP-1 monoclonal antibody is a human antibody, chimeric antibody or humanized antibody.
21. The method of claim 16 wherein said cartilagenous disorder is a degenerative cartilagenous disorder.
22. The method of claim 16 wherein said cartilagenous disorder is an articular cartilagenous diorder.
23. The method of claim 22 wherein said articular cartilagenous disorder is osteoarthritis or rheumatoid arthritis.
24. The method of claim 16 wherein said mammal is also treated using one or more surgical techniques.
25. The method of claim 24 wherein said effective amount of WISP antagonist is administered to the mammal prior to, after, and/or simultaneous with the surgical technique(s).
26. A kit or article of manufacture, comprising WISP antagonist and a carrier, excipient and/or stabilizer, and printed instructions for using said WISP antagonist to treat a cartilagenous disorder.

### BRIEF DESCRIPTION OF THE DRAWINGS

FIGS. 1A-E. In Situ Hybridization Analysis Of WISP-1 Expression During Mouse Development. Left panels show dark-field images and right panels show corresponding bright-field images. (A) Base of the skull dorsal of the oropharynx (*) at E12.5. At E15.5, WISP-1 is expressed in osteoblasts and mesenchymal cells adjacent to bones undergoing endochondral ossification (B, vertebras; C, ribs) and intramembranous ossification (D, ossification within palatal shelf of maxilla). WISP-1 expression was similarly distributed in human embryo lower limb (E, lateral border of head of tibia). Original magnification: ×100 (A); ×40 (B); ×200 (C); ×100 (D); ×200 (E).

FIGS. 2A-D. Immunofluorescent Localization Of WISP-1 In Rat Embryo E18. Differentiating osteoblasts lining the calvaria (A), femur (B), and ribs (C, D). S, skull; P, periosteum; C, cartilage primordium. Original magnification: ×100 (A); ×200 (B); ×200 (C); ×400 (D).

FIGS. 3A-J. WISP-1 Is Induced In Differentiating Osteoblasts. (A) WISP-1 expression in different cell types. WISP-1 (B, E, H) and osteocalcin expression (C, F, I) and alkaline phosphatase activity (D, G, J) in MC3T3-E1 cells after ascorbic acid treatment (B-D), in ST2 cells after BMP-2 treatment (E-G) and in C2C12 cells after BMP-2 treatment (H-J).

FIGS. 4A-F. In Situ WISP-1 Binding Analysis In Mouse Embryo. At E14, WISP-1 binding revealed an intense fluorescent signal associated with costal (A) and vertebral (B) condensed mesenchymal cells. At E17, WISP-1 bound to osteoblasts and perichondral mesenchyme of developing bones; mesenchyme surrounding cartilage primordium of rib (C), calvaria (D), mesenchyme surrounding cartilage primordium of distal part of radius (E, F), P, perichondrium; C, cartilage primordium. S, skull. Original magnification: ×200 (A); ×40 (B); ×100 (C); ×200 (D); ×200 (E); ×400 (F).

FIGS. 5A-B. WISP-1 Binding To Dedifferentiated Chondrocytes. The binding of WISP-1 to dedifferentiated primary porcine chondrocytes showed an irregular pattern associated with patches and point of focal adhesion (A). Intense staining was found at the point of contact of adjacent cells (B). Original magnification ×200.

FIGS. 6A-E. WISP-1 Represses Chondrogenic Differentiation Of ATDC5 Cells. A, Western blot analysis of WISP-1 production by the ATDC5/control, ATDC5/WISP-1L and ATDC5/WISP-1H cell lines. Saturation density (B) and photomicrograph (C) of ATDC5 cell lines grown to confluency. D, proliferation of ATDC5 (empty squares), ATDC5/control (filled squares), ATDC5/WISP-1L (empty circles) and ATDC5/WISP-1H cells (filled circles). E, Relative expression of collagen 2 in ATDC5/control, ATDC5/WISP-1L and ATDC5/WISP-1H cells before (black bars) and after induced chondrocytic differentiation by BMP-2 (gray bars) or GDF-5 (white bars).

FIGS. 7A-E. In Situ Hybridization Analysis Of WISP-1 Expression During Fracture Repair. Left panels show bright-field images and right panels show corresponding dark-field images. Photomicrographs showing the localization of WISP-1 expression at day 3 (A), 5 (B), 7 (C), 14 (D), 21 (E) and 28 (F) after fracture. Each image (magnification ×200) is oriented with the medullary cavity in the upper right; the cortex (*) and fracture callus (arrow heads) occupy the majority of the photomicrograph.

FIGS. 8A-8C show the encoding DNA (SEQ ID NO:2) and amino acid (SEQ ID NO:1) sequences for human WISP-1.

FIGS. 9A-B. WISP-1 promotes BMP-2-induced osteoblastic differentiation. C2C12 cells were transiently transfected with an empty vector (black bars) or WISP-1 expression construct (grey bars). Forty-eight hours after transfection, the culture media was replaced by media containing 5% FBS (A) or media containing 5% FBS and 300 ng/ml BMP-2 (2) and alkaline phosphatase activity was measured at the indicated time.

FIGS. 10A-B. WISP-1 knock-down represses osteoblastic differentiation. C2C12 cells were transiently tranfected with a vector expressing a control shRNA or a vector expressing a shRNA targeting WISP-1. Twenty four hours after transfection, the culture media was replaced by media containing 5% FBS or media containing 5% FBS and 300 ng/ml BMP-2 and WISP-1 expression (A) and alkaline phosphatase activity (B) was measured after 48 hours.

### DETAILED DESCRIPTION OF THE INVENTION

I. Definitions

The term “WISP polypeptide” refers to the family of native-sequence human and mouse WISP proteins and variants described herein whose genes are induced at least by Wnt-1. This term includes WISP-1, WISP-2, and WISP-3 and variants thereof. Such WISP-1, WISP-2 and WISP-3 proteins are described further below and in PCT application WO99/21998 published May 6, 1999 and in Pennica et al., Proc. Natl. Acad. Sci., 95:14717-14722 (1998).

The terms “WISP-1 polypeptide”, “WISP-1 homologue”, “WISP-1 orthologue” and grammatical variants thereof, as used herein, encompass native-sequence WISP-1 protein and variants (which are further defined herein). The WISP-1 polypeptide may be isolated from a variety of sources, such as from human tissue types or from another source, or prepared by recombinant or synthetic methods, or by any combination of these and similar techniques.

The terms “WISP-2 polypeptide”, “WISP-2 homologue”, “WISP-2 orthologue” “PRO261”, and “PRO261 polypeptide” and grammatical variants thereof, as used herein, encompass native-sequence WISP-2 protein and variants (which are further defined herein). The WISP-2 polypeptide may be isolated from a variety of sources, such as from human tissue types or from another source, or prepared by recombinant or synthetic methods, or by any combination of these and similar techniques.

The terms “WISP-3 polypeptide”, “WISP-3 homologue”, “WISP-3 orthologue” and grammatical variants thereof, as used herein, encompass native-sequence WISP-3 protein and variants (which are further defined herein). The WISP-3 polypeptide may be isolated from a variety of sources, such as from human tissue types or from another source, or prepared by recombinant or synthetic methods, or by any combination of these and similar techniques.

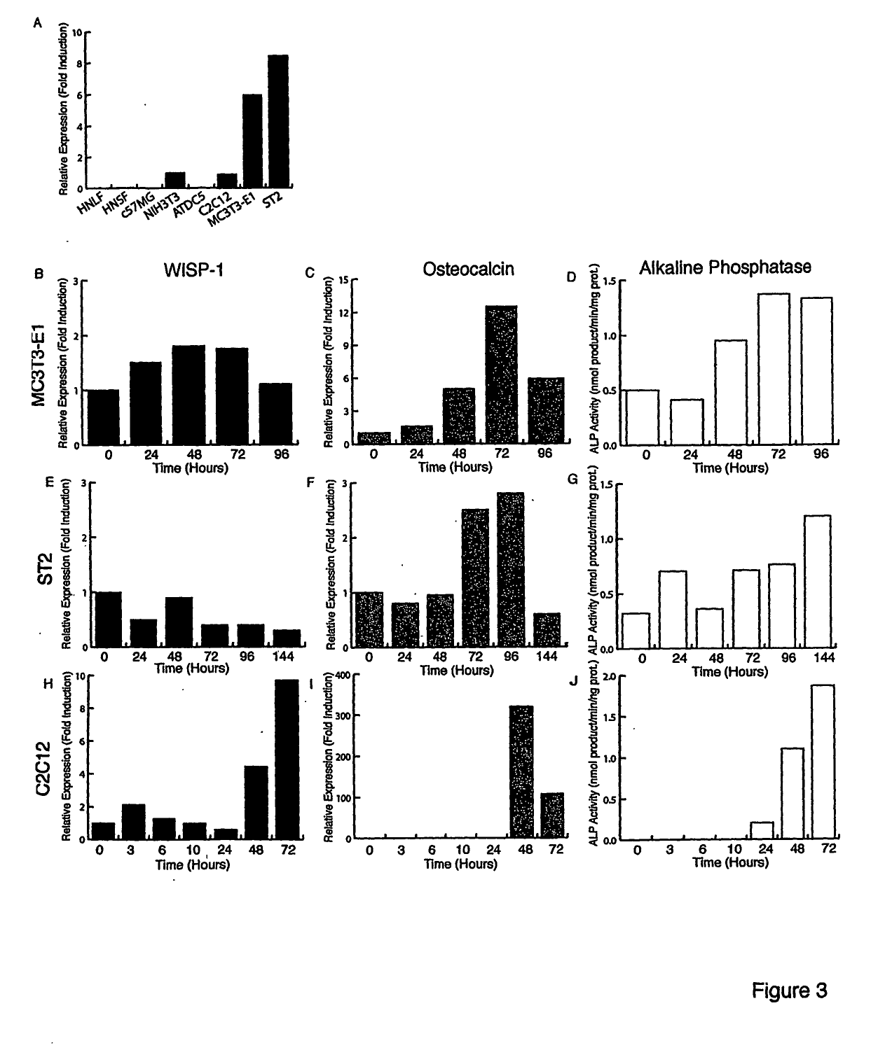

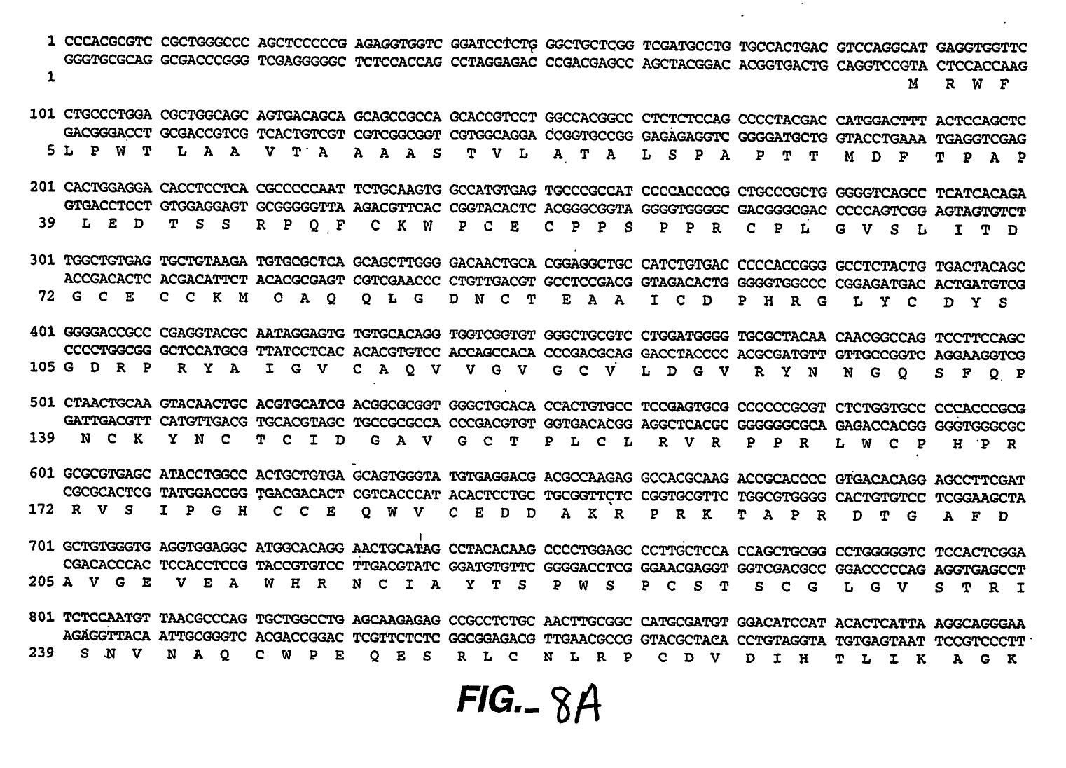

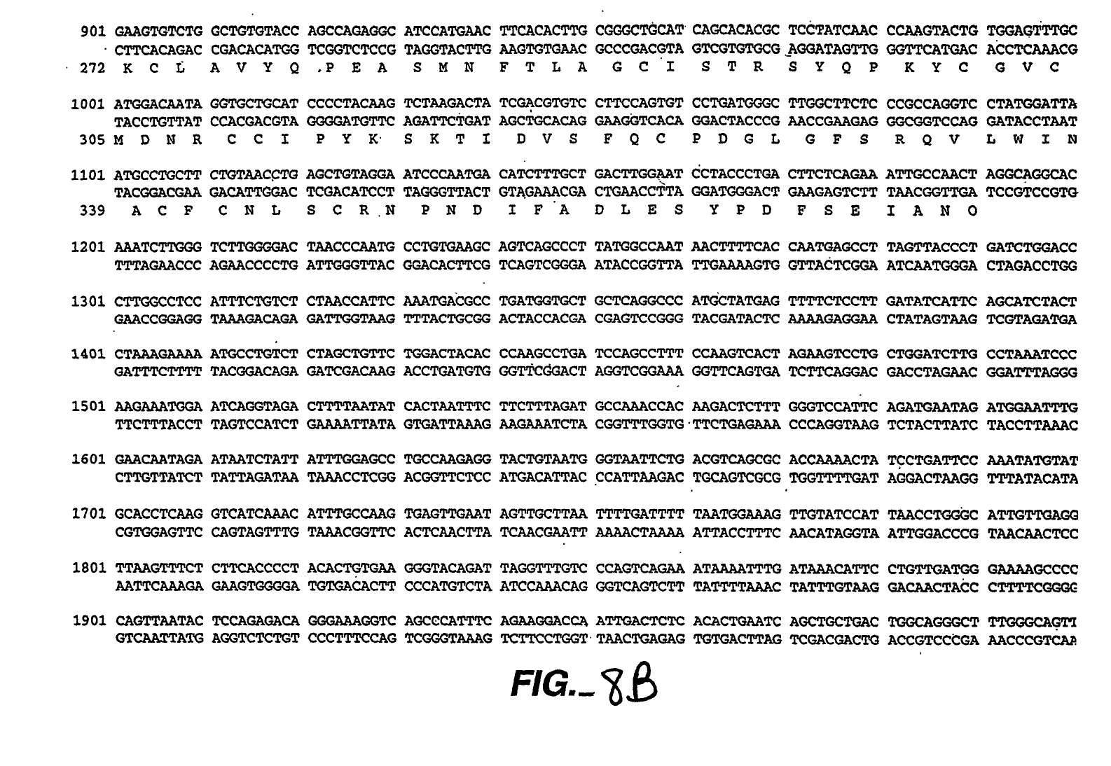

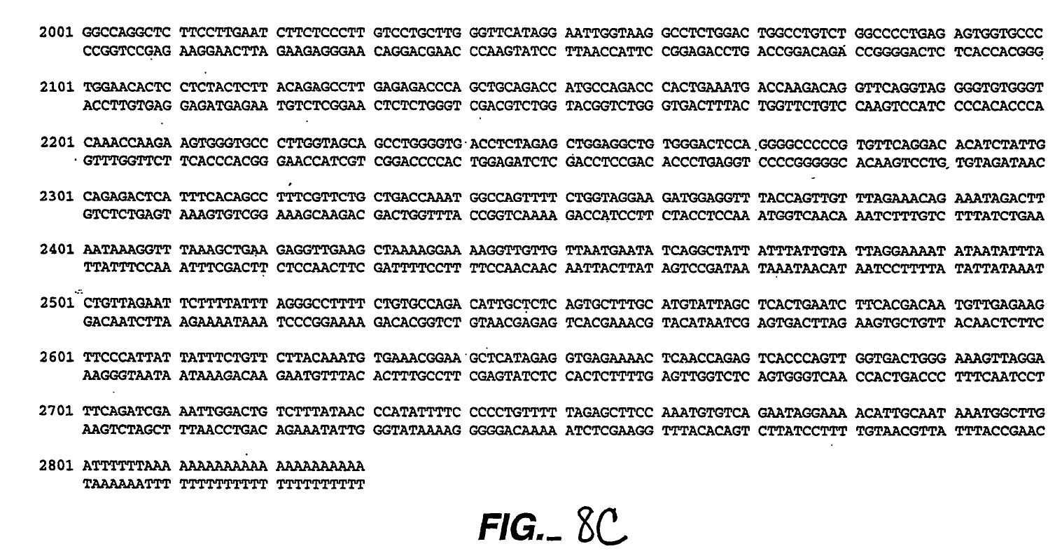

A “native-sequence WISP-1 polypeptide” comprises a polypeptide having the same amino acid sequence as a WISP-1 polypeptide derived from nature. Such native-sequence WISP-1 polypeptides can be isolated from nature or can be produced by recombinant or synthetic means. The term “native-sequence WISP-1 polypeptide” specifically encompasses naturally occurring truncated or secreted forms of a WISP-1 polypeptide disclosed herein, naturally occurring variant forms (e.g., alternatively spliced forms or splice variants), and naturally occurring allelic variants of a WISP-1 polypeptide. In one embodiment of the invention, the native-sequence WISP-1 polypeptide is a mature or full-length native-sequence human WISP-1 polypeptide comprising amino acids 23 to 367 of FIG. 8 herein (also provided previously in FIGS. 3A and 3B (SEQ ID NO:3) shown in WO99/21998 published May 6, 1999) or amino acids 1 to 367 of FIG. 8 herein (previously provided in FIGS. 3A and 3B (SEQ ID NO:4) shown in WO99/21998), respectively, with or without a N-terminal methionine. Optionally, the human WISP-1 polypeptide comprises the contiguous sequence of amino acids 23 to 367 or amino acids 1 to 367 of FIG. 8 herein. Optionally, the human WISP-1 polypeptide is encoded by a polynucleotide sequence having the coding nucleotide sequence as in ATCC deposit no. 209533.

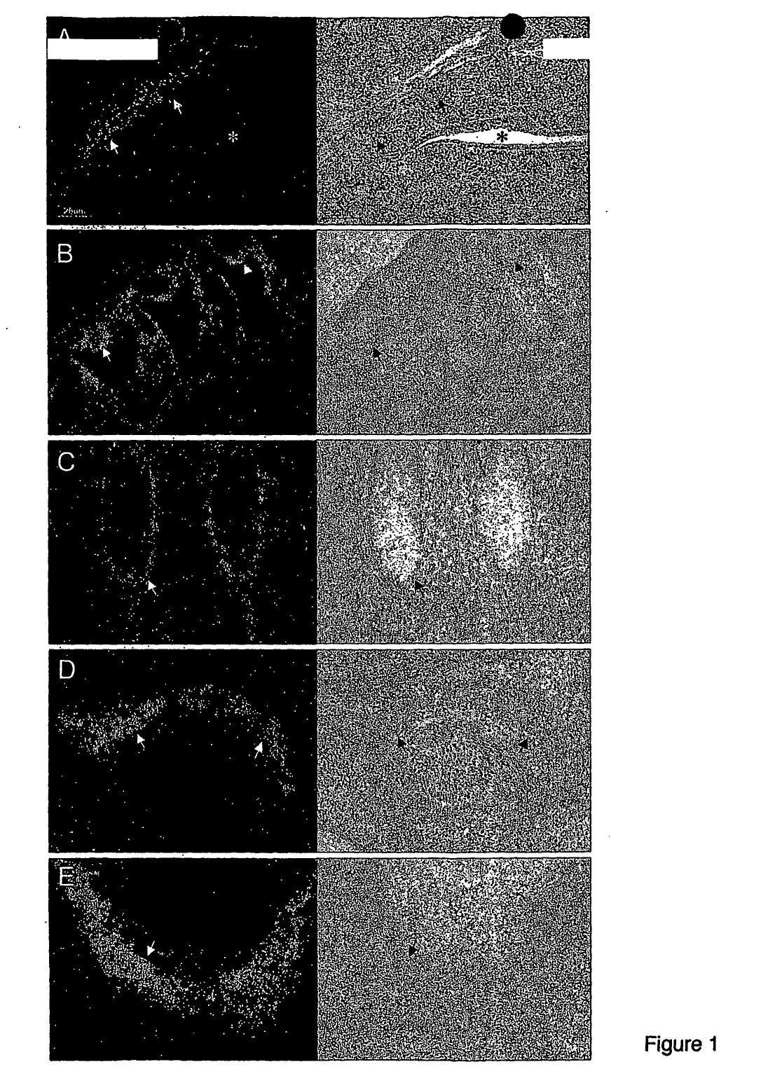

In another embodiment of the invention, the native-sequence WISP-1 polypeptide is the full-length or mature native-sequence human WISP-1 polypeptide comprising amino acids 23 to 367 or 1 to 367 of FIG. 8 herein wherein the valine residue at position 184 or the alanine residue at position 202 has/have been changed to an isoleucine or serine residue, respectively, with or without a N-terminal methionine. In another embodiment of the invention, the native-sequence WISP-1 polypeptide is the full-length or mature native-sequence human WISP-1 polypeptide comprising amino acids 23 to 367 or 1 to 367 of FIG. 8 herein wherein the valine residue at position 184 and the alanine residue at position 202 has/have been changed to an isoleucine or serine residue, respectively, with or without a N-terminal methionine. In another embodiment of the invention, the native-sequence WISP-1 polypeptide is a mature or full-length native-sequence mouse WISP-1 polypeptide comprising amino acids 23 to 367 of FIG. 8 herein (previously provided in FIG. 1 (SEQ ID NO:11) shown in WO99/21998), or amino acids 1 to 367 of FIG. 8 herein (previously provided in FIG. 1 (SEQ ID NO:12) shown in WO99/21998), respectively, with or without a N-terminal methionine.

In another embodiment of the invention, the native-sequence WISP-1 polypeptide is one which is encoded by a nucleotide sequence comprising one of the human WISP-1 splice or other native-sequence variants, including SEQ ID NOS:23, 24, 25, 26, 27, 28, or 29 shown in WO99/21998, with or without a N-terminal methionine.

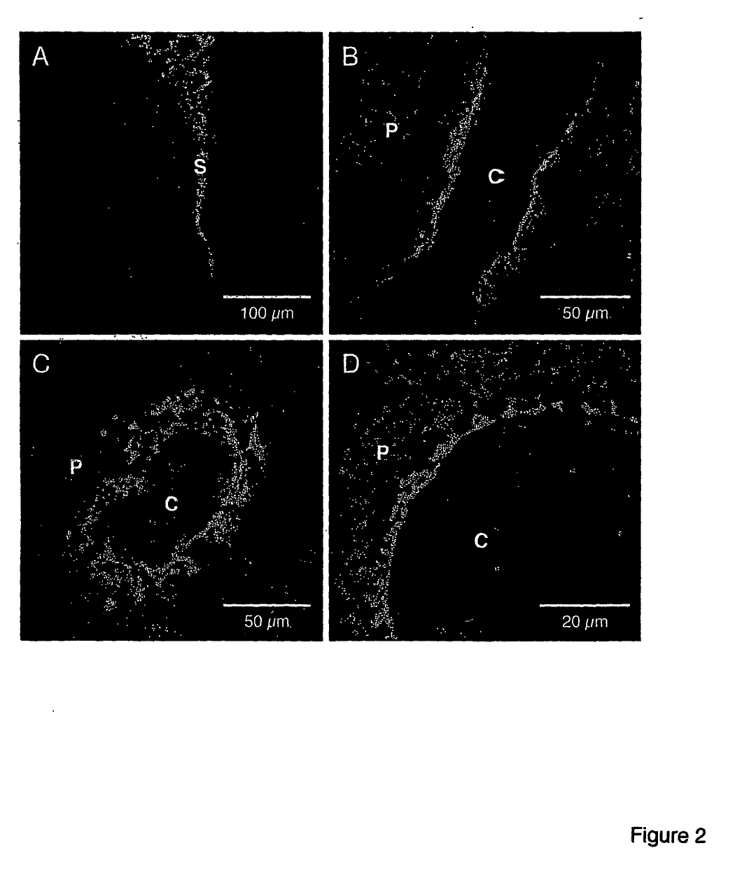

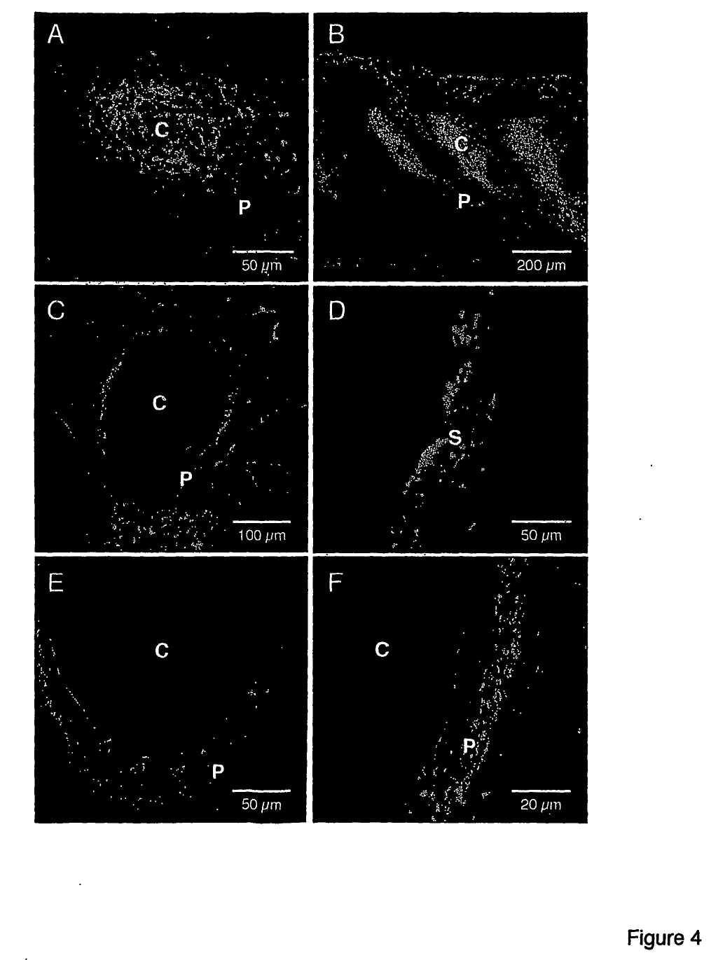

A “native-sequence WISP-2 polypeptide” or a “native-sequence PRO261 polypeptide” comprises a polypeptide having the same amino acid sequence as a WISP-2 polypeptide derived from nature. Such native-sequence WISP-2 polypeptides can be isolated from nature or can be produced by recombinant or synthetic means. The term “native-sequence WISP-2 polypeptide” specifically encompasses naturally occurring truncated or secreted forms of a WISP-2 polypeptide disclosed herein, naturally occurring variant forms (e.g., alternatively spliced forms or splice variants), and naturally occurring allelic variants of a WISP-2 polypeptide. In one embodiment of the invention, the native-sequence WISP-2 polypeptide is a mature or full-length native-sequence human WISP-2 polypeptide comprising amino acids 1-24 up to 250, previously provided in FIG. 4 (SEQ ID NOS:15, 16, and 56-77) shown in WO99/21998), including amino acids 24 to 250 and amino acids 1 to 250, with or without a N-terminal methionine. Optionally, the human WISP-2 polypeptide comprises the contiguous sequence of amino acids 24 to 250 or amino acids 1 to 250. Optionally, the human WISP-2 polypeptide is encoded by a polynucleotide sequence having the coding nucleotide sequence as in ATCC deposit no. 209391. In another embodiment of the invention, the native-sequence WISP-2 polypeptide is a mature or full-length native-sequence mouse WISP-2 polypeptide comprising amino acids 1-24 up to 251 of the FIG. 2 (SEQ ID NOS:19, 20, and 78-99) shown in WO99/21998, including amino acids 24 to 251 and amino acids 1 to 251 of the FIG. 2 (SEQ ID NOS:19 and 20, respectively) shown in WO99/21998, with or without a N-terminal methionine.

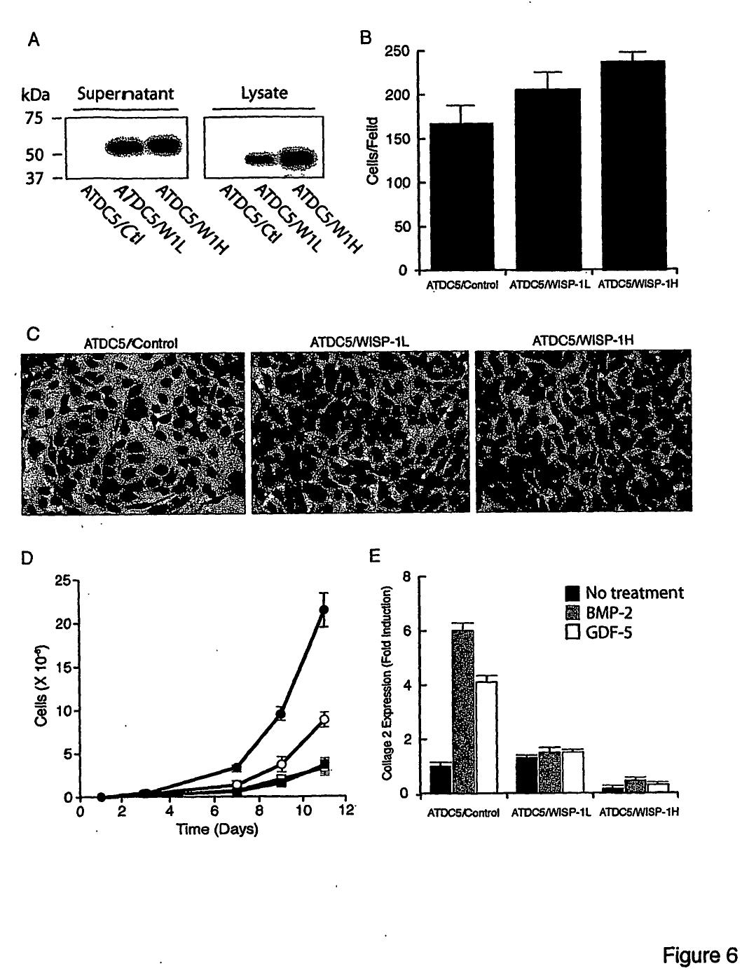

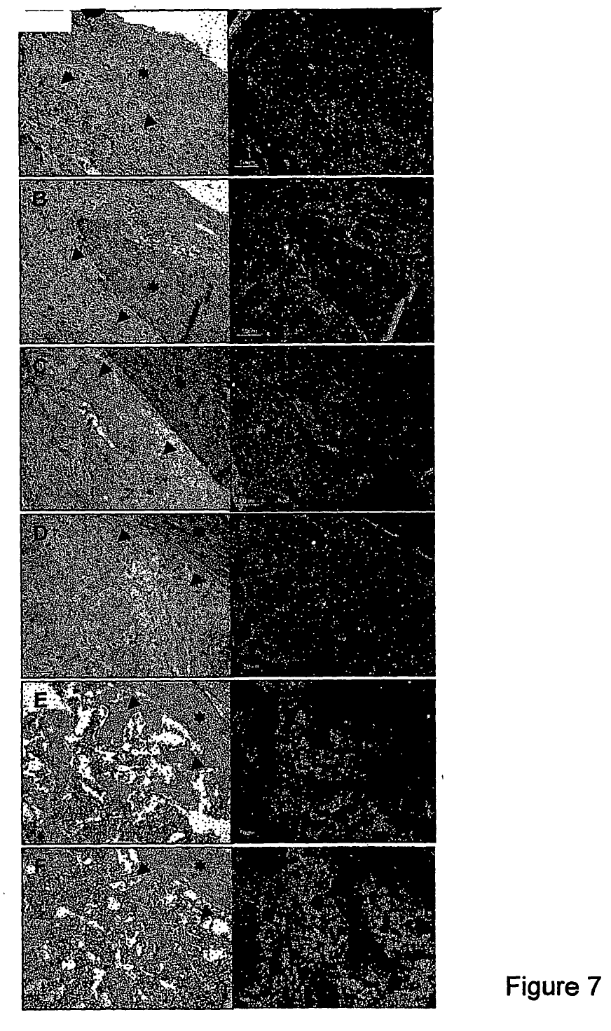

A “native-sequence WISP-3 polypeptide” comprises a polypeptide having the same amino acid sequence as a WISP-3 polypeptide derived from nature. Such native-sequence WISP-3 polypeptides can be isolated from nature or can be produced by recombinant or synthetic means. The term “native-sequence WISP-3 polypeptide” specifically encompasses naturally occurring truncated or other forms of a WISP-3 polypeptide disclosed herein, naturally occurring variant forms (e.g., alternatively spliced forms or splice variants), and naturally occurring allelic variants of a WISP-3 polypeptide. In one embodiment of the invention, the native-sequence WISP-3 polypeptide is a mature or full-length, native-sequence human WISP-3 polypeptide comprising amino acids 34 to 372 of previously provided in FIGS. 6A and 6B (SEQ ID NO:32) of WO99/21998) or amino acids 1 to 372 of previously provided in FIGS. 6A and 6B (SEQ ID NO:33) shown in WO99/21998), respectively, with or without a N-terminal methionine. In another embodiment of the invention, the native-sequence WISP-3 polypeptide is a mature or full-length, native-sequence human WISP-3 polypeptide comprising amino acids 16 to 354 of previously provided in FIGS. 7A and 7B (SEQ ID NO:36) shown in WO 99/21998) or amino acids 1 to 354 of previously provided in FIGS. 7A and 7B (SEQ ID NO:37) shown in WO99/21998), respectively, with or without a N-terminal methionine. Optionally, the human WISP-3 polypeptide comprises the contiguous sequence of amino acids 34 to 372 or amino acids 1 to 372. Optionally, the human WISP-3 polypeptide comprises the contiguous sequence of amino acids 16 to 354 or 1 to 354. Optionally, the human WISP-3 polypeptide is encoded by a polynucleotide sequence having the coding nucleotide sequence as in ATCC deposit no. 209707.

The term “WISP-1 variant” means an active WISP-1 polypeptide as defined below having at least about 80%, preferably at least about 85%, more preferably at least about 90%, most preferably at least about 95% amino acid sequence identity with human mature WISP-1 having the deduced amino acid sequence of amino acids 23 to 367 of human WISP-1 or the deduced amino acid sequence of amino acids 1 to 367 of FIG. 8. Such variants include, for instance, WISP-1 polypeptides wherein one or more amino acid residues are added to, or deleted from (i.e., fragments), the N- or C-terminus of the full-length or mature sequences of WISP-1, including variants from other species, but excludes a native-sequence WISP-1 polypeptide.

The term “WISP-2 variant” or “PRO261 variant” means an active WISP-2 polypeptide as defined below having at least about 80%, preferably at least about 85%, more preferably at least about 90%, most preferably at least about 95% amino acid sequence identity with human mature WISP-2 having the putative deduced amino acid sequence of amino acids 24 to 250, and/or with human full-length WISP-2 having the deduced amino acid sequence of amino acids 1 to 250. Such variants include, for instance, WISP-2 polypeptides wherein one or more amino acid residues are added to, or deleted from (i.e., fragments), the N- or C-terminus of the full-length and putative mature sequences of WISP-2, including variants from other species, but excludes a native-sequence WISP-2 polypeptide.

The term “WISP-3 variant” means an active WISP-3 polypeptide as defined below having at least about 80%, preferably at least about 85%, more preferably at least about 90%, most preferably at least about 95% amino acid sequence identity with human mature WISP-3 having the deduced amino acid sequence of amino acids 34 to 372, and/or with human full-length WISP-3 having the deduced amino acid sequence of amino acids 1 to 372, and/or with human mature WISP-3 having the deduced amino acid sequence of amino acids 16 to 354, or with human full-length WISP-3 having the deduced amino acid sequence of amino acids 1 to 354. Such variants include, for instance, WISP-3 polypeptides wherein one or more amino acid residues are added to, or deleted from (i.e., fragments), the N- or C-terminus of the full-length or mature sequences of WISP-3, including variants from other species, but excludes a native-sequence WISP-3 polypeptide.

“Percent (%) amino acid sequence identity” with respect to the WISP polypeptide sequences identified herein is defined as the percentage of amino acid residues in a candidate sequence that are identical with the amino acid residues in such WISP sequences identified herein, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are obtained by using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

“Stringent conditions” are those that (1) employ low ionic strength and high temperature for washing, 0.015 M sodium chloride/0.0015 M sodium citrate/0.1% sodium dodecyl sulfate at 50° C.; (2) employ during hybridization a denaturing agent, such as formamide, 50% (vol/vol) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC and 0.1% SDS; or (4) employ a buffer of 10% dextran sulfate, 2×SSC (sodium chloride/sodium citrate), and 50% formamide at 55° C., followed by a high-stringency wash consisting of 0.1×SSC containing EDTA at 55° C.

“Moderately stringent conditions” are described in Sambrook et al., Molecular Cloning: A Laboratory Manual (New York: Cold Spring Harbor Laboratory Press, 1989), and include the use of a washing solution and hybridization conditions (e.g., temperature, ionic strength, and percent SDS) less stringent than described above. An example of moderately stringent conditions is a condition such as overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc., as necessary to accommodate factors such as probe length and the like.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the WISP natural environment will not be present. Ordinarily, however, isolated polypeptide will be prepared by at least one purification step.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

A “liposome” is a small vesicle composed of various types of lipids, phospholipids and/or surfactant which is useful for delivery of a drug (such as the WISP polypeptides and WISP variants disclosed herein) to a mammal. The components of the liposome are commonly arranged in a bilayer formation, similar to the lipid arrangement of biological membranes.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” in the context of the WISP polypeptides or WISP variants of the invention refers to form(s) of proteins of the invention which retain the biologic and/or immunologic activities of a native or naturally-occurring WISP polypeptide, wherein “biological” activity refers to a biological function (either inhibitory or stimulatory). caused by a native or naturally-occurring WISP polypeptide other than the ability to serve as an antigen in the production of an antibody against an antigenic epitope possessed by a native or naturally-occurring polypeptide of the invention. Similarly, an “immunological” activity refers to the ability to serve as an antigen in the production of an antibody against an antigenic epitope possessed by a native or naturally-occurring polypeptide of the invention.

“Biological activity” in the context of a WISP antagonist herein is used to refer to the ability of such molecules to inhibit or block the effects of WISP-1 on chondrocyte differentiation (i.e., differentiation of a precursor cell into a mature chondrocyte). Optionally, the cartilage is articular cartilage and the regeneration and/or destruction of the cartilage is associated with an injury or a degenerative cartilagenous disorder. For example, such biological activity may be quantified by in vitro chondrocyte differentiation assays and gene expression analysis.

The term “WISP-1 antagonist” refers to any molecule that partially or fully blocks, inhibits, or neutralizes a biological activity of WISP-1 and include but are not limited to, antibodies, immunoadhesins, WISP-1 immunoadhesins, WISP-1 fusion proteins, covalently modified forms of WISP-1, WISP-1 variants and fusion proteins thereof, WISP-1 antibodies, and higher oligomer forms of WISP-1 (dimers, aggregates) or homo- or heteropolymer forms of WISP-1. To determine whether a WISP-1 antagonist molecule partially or fully blocks, inhibits or neutralizes a biological activity of WISP-1, assays may be conducted to assess the effect(s) of the antagonist molecule on, for example, various cells (as described in the Examples). Preferably, the WISP-1 antagonists employed in the methods described herein will be capable of blocking, inhibiting or neutralizing WISP-1 effects on chondrocyte differentiation, which may optionally be determined in assays such as described herein.

The term “antibody” is used in the broadest sense and specifically covers, for example, single monoclonal antibodies, antibody compositions with polyepitopic specificity, single chain antibodies, and fragments of antibodies. “Antibody” as used herein includes intact immunoglobulin or antibody molecules, polyclonal antibodies, multispecific antibodies (i.e., bispecific antibodies formed from at least two intact antibodies) and immunoglobulin fragments (such as Fab, F(ab′)₂, or Fv), so long as they exhibit any of the desired antagonistic properties described herein.

Antibodies are typically proteins or polypeptides which exhibit binding specificity to a specific antigen. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains [Chothia et al., J. Mol. Biol., 186:651-663 (1985); Novotny and Haber, Proc. Natl. Acad. Sci. USA, 82:4592-4596 (1985)]. The light chains of antibodies from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called alpha, delta, epsilon, gamma, and mu, respectively.

“Antibody fragments” comprise a portion of an intact antibody, generally the antigen binding or variable region of the intact antibody. Examples of antibody fragments include Fab, Fab′, F(ab′)2, and Fv fragments, diabodies, single chain antibody molecules, and multispecific antibodies formed from antibody fragments.

The term “variable” is used herein to describe certain portions of the variable domains which differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies [see Kabat, E. A. et al., Sequences of Proteins of Immunological Interest, National Institutes of Health, Bethesda, Md. (1987)]. The constant domains are not involved directly in binding an antibody to an antigen, but exhibit various effector functions, such as participation of the antibody in antibody-dependent cellular toxicity.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The monoclonal antibodies herein include chimeric, hybrid and recombinant antibodies produced by splicing a variable (including hypervariable) domain of the antibody of interest with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity or properties. See, e.g. U.S. Pat. No. 4,816,567 and Mage et al., in Monoclonal Antibody Production Techniques and Applications, pp. 79-97 (Marcel Dekker, Inc.: New York, 1987).

Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature, 256:495 (1975), or may be made by recombinant DNA methods such as described in U.S. Pat. No. 4,816,567. The “monoclonal antibodies” may also be isolated from phage libraries generated using the techniques described in McCafferty et al., Nature, 348:552-554 (1990), for example.

“Humanized” forms of non-human (e.g. murine) antibodies are specific chimeric immunoglobulins, immunoglobulin chains, or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin.

A “human antibody” is one which possesses an amino acid sequence which corresponds to that of an antibody produced by a human and/or has been made using any of the techniques for making human antibodies known in the art or as disclosed herein. This definition of a human antibody includes antibodies comprising at least one human heavy chain polypeptide or at least one human light chain polypeptide, for example an antibody comprising murine light chain and human heavy chain polypeptides. Human antibodies can be produced using various techniques known in the art. In one embodiment, the human antibody is selected from a phage library, where that phage library expresses human antibodies (Vaughan et al. Nature Biotechnology, 14:309-314 (1996): Sheets et al. PNAS, (USA) 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology, 10: 779-783 (1992); Lonberg et al., Nature, 368: 856-859 (1994); Morrison, Nature, 368:812-13 (1994); Fishwild et al., Nature Biotechnology, 14: 845-51 (1996); Neuberger, Nature Biotechnology, 14: 826 (1996); Lonberg and Huszar, Intern. Rev. Immunol., 13:65-93 (1995). Alternatively, the human antibody may be prepared via immortalization of human B lymphocytes producing an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region (using herein the numbering system according to Kabat et al., supra). The Fc region of an immunoglobulin generally comprises two constant domains, a CH2 domain and a CH3 domain, and optionally comprises a CH4 domain.

By “Fc region chain” herein is meant one of the two polypeptide chains of an Fc region.

The “CH2 domain” of a human IgG Fc region (also referred to as “Cγ2” domain) usually extends from an amino acid residue at about position 231 to an amino acid residue at about position 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985). The CH2 domain herein may be a native sequence CH2 domain or variant CH2 domain.

The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protroberance” in one chain thereof and a corresponding introduced “cavity” in the other chain thereof; see U.S. Pat. No. 5,821,333). Such variant CH3 domains may be used to make multispecific (e.g. bispecific) antibodies as herein described.

“Hinge region” is generally defined as stretching from about Glu216, or about Cys226, to about Pro230 of human IgGl (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions. The hinge region herein may be a native sequence hinge region or a variant hinge region. The two polypeptide chains of a variant hinge region generally retain at least one cysteine residue per polypeptide chain, so that the two polypeptide chains of the variant hinge region can form a disulfide bond between the two chains. The preferred hinge region herein is a native sequence human hinge region, e.g. a native sequence human IgG1 hinge region.

A “functional Fc region” possesses at least one “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays known in the art for evaluating such antibody effector functions.

A “native sequence Fc region” comprises an amino acid sequence identical to the amino acid sequence of a Fc region found in nature. A “variant Fc region” comprises an amino acid sequence which differs from that of a native sequence Fc region by virtue of at least one amino acid modification. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably at least about 90% sequence identity therewith, more preferably at least about 95% sequence identity therewith.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol., 9:457-92 (1991). To assess ADCC activity of a molecule of interest, an in vitro ADCC assay, such as that described in U.S. Pat. Nos. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA), 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

The terms “Fc receptor” and “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol., 15:203-234 (1997)). FcRs are reviewed in Ravetch and Kinet, Annu. Rev. Immunol., 9:457-92 (1991); Capel et al., Immunomethods, 4:25-34 (1994); and de Haas et al., J. Lab. Clin. Med., 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol., 117:587 (1976); and Kim et al., J. Immunol., 24:249 (1994)).

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202:163 (1996), may be performed.

An “affinity matured” antibody is one with one or more alterations in one or more CDRs thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology, 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene, 169:147-155 (1995); Yelton et al. J. Immunol., 155:1994-2004 (1995); Jackson et al., J. Immunol., 154(7):3310-9 (1995); and Hawkins et al, J. Mol. Biol., 226:889-896 (1992).

The term “immunospecific” as used in “immunospecific binding of antibodies” for example, refers to the antigen specific binding interaction that occurs between the antigen-combining site of an antibody and the specific antigen recognized by that antibody.

The term “cartilagenous disorder” refers generally to a disease manifested by symptoms of pain, stiffness and/or limitation of motion of the affected body parts. Included within the scope of “cartilagenous disorder” is “degenerative cartilagenous disorder”—a disorder characterized, at least in part, by degeneration or metabolic derangement of connective tissues of the body, including not only the joints or related structures, including muscles, bursae (synovial membrane), tendons and fibrous tissue, but also the growth plate. In one embodiment, the term includes “articular cartilage disorder” which is characterized by disruption of the smooth articular cartilage surface and degradation of the cartilage matrix. Additional pathologies include nitric oxide production, and inhibition or reduction of matrix synthesis.

Included within the scope of “articular cartilage disorder” are osteoarthritis (OA) and rheumatoid arthritis (RA). OA is characterized by localized asymmetric destruction of the cartilage commensurate with palpable bony enlargements at the joint margins. OA typically affects the interphalangeal joints of the hands, the first carpometacarpal joint, the hips, the knees, the spine, and some joints in the midfoot, while large joints, such as the ankles, elbows and shoulders tend to be spared. OA can be associated with metabolic diseases such as hemochromatosis and alkaptonuria, developmental abnormalities such as developmental dysplasia of the hips (congenital dislocation of the hips), limb-length discrepancies, including trauma and inflammatory arthritides such as gout, septic arthritis, and neuropathic arthritis. OA may also develop after extended biomechanical instability, such as resulting from sports injury or obesity.

Rheumatoid arthritis (RA) is a systemic, chronic, autoimmune disorder characterized by symmetrical synovitis of the joint and typically affects small and large diarthroid joints alike. As RA progresses, symptoms may include fever, weight loss, thinning of the skin, multiorgan involvement, scleritis, corneal ulcers, the formation of subcutaneous or subperiosteal nodules and even premature death. The symptoms of RA often appear during youth and can include vasculitis, atrophy of the skin and muscle, subcutaneous nodules, lymphadenopathy, splenomegaly, leukopaenia and chronic anaemia.

Furthermore, the term “degenerative cartilagenous disorder” may include systemic lupus erythematosus and gout, amyloidosis or Felty's syndrome. Additionally, the term covers the cartilage degradation and destruction associated with psoriatic arthritis, osteoarthrosis, acute inflammation (e.g., yersinia arthritis, pyrophosphate arthritis, gout arthritis (arthritis urica), septic arthritis), arthritis associated with trauma, ulcerative colitis (e.g., Crohn's disease), multiple sclerosis, diabetes (e.g., insulin-dependent and non-insulin dependent), obesity, giant cell arthritis and Sjögren's syndrome.

Examples of other immune and inflammatory diseases, at least some of which may be treatable by the methods of the invention include, juvenile chronic arthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis) autoimmune inflammatory diseases (e.g., allergic encephalomyelitis, multiple sclerosis, insulin-dependent diabetes mellitus, autoimmune uveoretinitis, thyrotoxicosis, scleroderma, systemic lupus erythematosus, rheumatoid arthritis, inflammatory bowel disease (e.g., Crohn's disease, ulcerative colitis, regional enteritis, distal ileitis, granulomatous enteritis, regional ileitis, terminal ileitis), autoimmune thyroid disease, pernicious anemia) and allograft rejection, diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis, Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases including viral diseases such as AIDS (HIV infection), hepatitis A, B, C, D, and E, herpes, etc., bacterial infections, fungal infections, protozoal infections, parasitic infections, and respiratory syncytial virus, human immunodeficiency virus, etc.) and allergic disorders, such as anaphylactic hypersensitivity, asthma, allergic rhinitis, atopic dermatitis, vernal conjunctivitis, eczema, urticaria and food allergies, etc.

“Treatment” is an intervention performed with the intention of preventing the development or altering the pathology of a disorder. Accordingly, “treatment” refers to both therapeutic treatment and prophylactic or preventative measures, wherein the object is to prevent or slow down (lessen) the targeted pathological condition or disorder. Those in need of treatment include those already with the disorder as well as those in which the disorder is to be prevented. In treatment of a degenerative cartilagenous disorder, a therapeutic agent may directly decrease or increase the magnitude of response of a pathological component of the disorder, or render the disease more susceptible to treatment by other therapeutic agents, e.g. antibiotics, antifungals, anti-inflammatory agents, chemotherapeutics, etc.

The term “effective amount” is the minimum concentration of WISP antagonist which causes, induces or results in either a detectable improvement or repair of cartilage. Furthermore a “therapeutically effective amount” is the minimum concentration (amount) of WISP antagonist administered to a mammal which would be effective in at least attenuating a pathological symptom (e.g. causing, inducing or resulting in either a detectable improvement or repair in cartilage) which occurs as a result of injury or a degenerative cartilagenous disorder.

“Cartilage agent” may be a growth factor, cytokine, small molecule, antibody, piece of RNA or DNA, virus particle, peptide, or chemical having a beneficial effect upon cartilage, including peptide growth factors, catabolism antagonists and osteo-, synovial- or anti-inflammatory factors. Alternatively, “cartilage agent” may be a peptide growth factor—such as any of the fibroblast growth factors (e.g., FGF-1, FGF-2, . . . FGF-21, etc.), IGF's (I and II), TGF-βs (1-3), BMPs (1-7), or members of the epidermal growth factor family such as EGF, HB-EGF, TGF-β—which could enhance the intrinsic reparative response of cartilage, for example by altering proliferation, differentiation, migration, adhesion, or matrix production by chondrocytes. Alternatively, a “cartilage agent” may be a factor which antagonizes the catabolism of cartilage (e.g., IL-1 receptor antagonist (IL-1ra), NO inhibitors, IL1-beta convertase (ICE) inhibitors, factors which inhibit activity of IL-6, IL-8, LIF, IFN-gamma, or TNF-alpha activity, tetracyclines and variants thereof, inhibitors of apoptosis, MMP inhibitors, aggrecanase inhibitors, inhibitors of serine and cysteine proteinases such as cathepsins and urokinase or tissue plasminogen activator (uPA and tPA). Alternatively still, cartilage agent includes factors which act indirectly on cartilage by affecting the underlying bone (i.e., osteofactors, e.g. bisphosphonates or osteoprotegerin) or the surrounding synovium (i.e., synovial factors) or anti-inflammatory factors (e.g., anti-TNF-alpha (including anti-TNF-alpha antibodies such as Remicade®, as well as TNF receptor immunoadhesins such as Enbrel®), IL-1ra, IL-4, IL-10, IL-13, NSAIDs). For a review of cartilage agent examples, please see Martel-Pelletier et al., Front. Biosci. 4: d694-703 (1999); Hering, T. M., Front. Biosci. 4: d743-761 (1999).

“Chronic” administration refers to administration of the factor(s) in a continuous mode as opposed to an acute mode, so as to maintain the initial therapeutic effect (activity) for an extended period of time. “Intermittent” administration is treatment that is done not consecutively without interruption, but rather is cyclic in nature.

“Mammal” for purposes of treatment refers to any animal classified as a mammal, including humans, domestic and farm animals, and zoo, sports, or pet animals, such as dogs, horses, cats, cattle, pigs, hamsters, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®, hyaluronic acid (HA).

II. Methods and Compositions of the Invention

During vertebrate embryogenesis, most skeletal elements are first formed by cartilagenous templates that are progressively replaced by bone in a process called endochondral ossification (For review articles, see, e.g.,—Karsenty, Nature, 423: 316-318 (2003); Karsenty and Wagner, Dev Cell, 2: 389-406 (2002); Kronenberg, Nature, 423: 332-336 (2003); Mariani and Martin, Nature, 423: 319-325 (2003). This process begins with the proliferation and condensation of committed osteochondroprogenitor mesenchymal cells into aggregates. Cells at the center of these aggregates differentiate into chondrocytes and initiate the synthesis of cartilage. Spindle shaped cells surrounding the cartilage templates align longitudinally to form the perichondrium that separates the chondrocytes from the adjacent tissue. The chondrocytes at the distal ends of the templates continue to proliferate while the cells in the central region of the cartilage elements exit the cell cycle and become hypertrophic. Differentiation into hypertrophic chondrocytes is accompanied by the differentiation of the mesenchymal cells of the perichondrium into osteoblasts. Osteoblasts are responsible for the deposition of bone matrix forming the bone collar surrounding the hypertrophic region of the cartilage. The invasion of hypertrophic cartilage by blood vessels and osteogenic cells results in the replacement of the cartilage by bone. Alternately, in some skeletal elements, especially the flat bones of the skull, the osteochondroprogenitor cells bypass the cartilagenous template formation and directly differentiate into osteoblasts. This process is called intramembranous ossification. The Wnt/β-catenin pathway constitutes one of the molecular mechanisms regulating several aspect of bone development including chondrocyte and osteoblast differentiation and joint formation. Gong et al., Cell, 107: 513-523 (2001); Hartmann et al., Development, 127: 3141-3159 (2000); Hartmann and Tabin, Cell, 104: 341-351 (2001); Rudnicki and Brown, Dev Biol, 185:104-118 (1997).

To investigate the role of WISP-1 in osteogenic processes, its tissue and cellular expression was characterized and its activity in chondroblastic and osteoblastic cell culture models was evaluated. During embryonic development, WISP-1 expression appeared to be restricted to osteoblasts and to osteoblastic progenitor cells of the perichondral mesenchyme. In vitro, WISP-1 induction occurred early during osteoblastic differentiation and was maintained in mature osteoblasts. Using in situ and cell binding analysis, WISP-1 interaction with perichondral mesenchyme and undifferentiated chondrocytes was demonstrated. The effect of WISP-1 was evaluated on chondrocyte progenitors by generating stably transfected mouse chondrocytic cell lines. In these cells, WISP-1 increased proliferation and saturation density but repressed chondrocytic differentiation. Because of the similarity between skeletogenesis and bone healing, WISP-1 spatiotemporal expression in a fracture repair model was also analyzed. WISP-1 expression recapitulated the pattern observed during skeletal development. Such experiments are further described in the Examples section below. The data demonstrated that WISP-1 is an osteoblastic factor that regulates chondrocytic differentiation and proliferation and it is believed that WISP-1 plays an important regulatory role during bone development and fracture repair.

In accordance with the methods of the present invention, various WISP antagonists may be employed for treatment of cartilage disorders as well as various other immune and immune related conditions. Such WISP antagonists include WISP-1 antibodies and WISP-1 variants thereof (as well as fusion proteins thereof such as epitope tagged forms or Ig-fusion constructs thereof). The WISP antagonists may be used in vivo as well as ex vivo. Optionally, the WISP antagonists are used in the form of pharmaceutical compositions, described in further detail below.

It is contemplated that WISP-1 polypeptide variants can be prepared. WISP-1 variants can be prepared by introducing appropriate nucleotide changes into the encoding DNA, and/or by synthesis of the desired polypeptide. Those skilled in the art will appreciate that amino acid changes may alter post-translational processes of the WISP-1 polypeptide, such as changing the number or position of glycosylation sites or altering the membrane anchoring characteristics.

Variations in the WISP-1 polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the polypeptide that results in a change in the amino acid sequence as compared with the native sequence polypeptide. Optionally the variation is by substitution of at least one amino acid with any other amino acid in one or more of the domains of the WISP-1 polypeptide. Guidance in determining which amino acid residue may be inserted, substituted or deleted without adversely affecting the desired activity may be found by comparing the sequence of the WISP-1 polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

WISP-1 polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the WISP-1 polypeptide.

WISP-1 polypeptide fragments may be prepared by any of a number of conventional techniques. Desired peptide fragments may be chemically synthesized. An alternative approach involves generating polypeptide fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR.

In particular embodiments, conservative substitutions of interest are shown in the Table below under the heading of preferred substitutions. If such substitutions result in a change in biological activity, then more substantial changes, denominated exemplary substitutions in the Table, or as further described below in reference to amino acid classes, are introduced and the products screened.

TABLE

| Original Residue | Exemplary Substitutions | Preferred Substitutions |
|---|---|---|
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | |
| | norleucine | leu |
| Leu (L) | norleucine; ile; val; | |
| | met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | |
| | ala; norleucine | leu |

Substantial modifications in function or immunological identity of the WISP-1 polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the WISP-1 polypeptide variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244:1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

Any cysteine residue not involved in maintaining the proper conformation of the WISP-1 polypeptide also may be substituted, generally with serine, to improve the oxidative stability of the molecule and prevent aberrant crosslinking. Conversely, cysteine bond(s) may be added to the WISP-1 polypeptide to improve its stability.

The description below relates primarily to production of WISP-1 polypeptides by culturing cells transformed or transfected with a vector containing WISP-1 polypeptide-encoding nucleic acid. It is, of course, contemplated that alternative methods, which are well known in the art, may be employed to prepare WISP-1 polypeptides. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the WISP-1 polypeptide may be chemically synthesized separately and combined using chemical or enzymatic methods to produce the desired WISP-1 polypeptide. The methods and techniques described are similarly applicable to production of WISP-1 variants, modified forms of WISP-1 and WISP-1 antibodies.

1. Isolation of DNA Encoding WISP-1 Polypeptide

DNA encoding WISP-1 polypeptide may be obtained from a cDNA library prepared from tissue believed to possess the WISP-1 polypeptide mRNA and to express it at a detectable level. Accordingly, human WISP-1 polypeptide DNA can be conveniently obtained from a cDNA library prepared from human tissue. The WISP-1 polypeptide-encoding gene may also be obtained from a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding WISP-1 polypeptide is to use PCR methodology [Sambrook et al., supra; Dieffenbach et al., PCR Primer: A Laboratory Manual (Cold Spring Harbor Laboratory Press, 1995)].

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected CDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for WISP-1 polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for WISP-1 polypeptide-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated WISP-1 polypeptide are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for WISP-1 polypeptide production and cultured in conventional nutrient media modified as appropriate for inducing promoters, selecting transformants, or amplifying the genes encoding the desired sequences.

3. Selection and Use of a Replicable Vector

The nucleic acid (e.g., cDNA or genomic DNA) encoding WISP-1 polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The WISP-1 may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the WISP-1 polypeptide-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the WISP-1 polypeptide-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the WISP-1 polypeptide-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding WISP polypeptide.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

WISP polypeptide transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the WISP-1 polypeptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the WISP-1 polypeptide coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding WISP-1 polypeptide.

Still other methods, vectors, and host cells suitable for adaptation to the synthesis of WISP polypeptide in recombinant vertebrate cell culture are described in Gething et al., Nature, 293:620-625 (1981); Mantei et al., Nature, 281:40-46 (1979); EP 117,060; and EP 117,058.

4. Culturing the Host Cells

The host cells used to produce the WISP polypeptide of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

5. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), semi-quantitative PCR, DNA array gene expression analysis, or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence WISP polypeptide or against a synthetic peptide based on the DNA sequences provided herein or against exogenous sequence fused to WISP DNA and encoding a specific antibody epitope.

6. Purification of WISP Polypeptide

Forms of WISP polypeptide may be recovered from culture medium or from host cell lysates. If membrane-bound, it can be released from the membrane using a suitable detergent solution (e.g. Triton-X 100) or by enzymatic cleavage. Cells employed in expression of WISP-1 polypeptide can be disrupted by various physical or chemical means, such as freeze-thaw cycling, sonication, mechanical disruption, or cell lysing agents.

It may be desired to purify WISP-1 polypeptide from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the WISP-1 polypeptide. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular WISP-1 polypeptide produced.

Soluble forms of WISP-1 may be employed as antagonists in the methods of the invention. Such soluble forms of WISP-1 may comprise modifications, as described below (such as by fusing to an immunoglobulin, epitope tag or leucine zipper). Immunoadhesin molecules are further contemplated for use in the methods herein. WISP-1 immunoadhesins may comprise various forms of WISP-1, such as the full length polypeptide as well as soluble forms of the WISP-1 or a fragment thereof. In particular embodiments, the molecule may comprise a fusion of the WISP-1 polypeptide with an immunoglobulin or a particular region of an immunoglobulin. For a bivalent form of the immunoadhesin, such a fusion could be to the Fc region of an IgG molecule. The Ig fusions preferably include the substitution of a soluble (transmembrane domain deleted or inactivated) form of the polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions, see also U.S. Pat. No. 5,428,130 issued Jun. 27, 1995 and Chamow et al., TIBTECH, 14:52-60 (1996).

The simplest and most straightforward immunoadhesin design combines the binding domain(s) of the adhesin (e.g. the WISP-1) with the Fc region of an immunoglobulin heavy chain. Ordinarily, when preparing the immunoadhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, C_{H}2 and C_{H}3 domains of the constant region of an immunoglobulin heavy chain. Fusions are also made to the C-terminus of the Fc portion of a constant domain, or immediately N-terminal to the C_{H}1 of the heavy chain or the corresponding region of the light chain. The precise site at which the fusion is made is not critical; particular sites are well known and may be selected in order to optimize the biological activity, secretion, or binding characteristics of the immunoadhesin.

In a preferred embodiment, the adhesin sequence is fused to the N-terminus of the Fc region of immunoglobulin G₁ (IgG₁). It is possible to fuse the entire heavy chain constant region to the adhesin sequence. However, more preferably, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In a particularly preferred embodiment, the adhesin amino acid sequence is fused to (a) the hinge region and C_{H}2 and C_{H}3 or (b) the C_{H}1, hinge, C_{H}2 and C_{H}3 domains, of an IgG heavy chain.

For bispecific immunoadhesins, the immunoadhesins are assembled as multimers, and particularly as heterodimers or heterotetramers. Generally, these assembled immunoglobulins will have known unit structures. A basic four chain structural unit is the form in which IgG, IgD, and IgE exist. A four chain unit is repeated in the higher molecular weight immunoglobulins; IgM generally exists as a pentamer of four basic units held together by disulfide bonds. IgA globulin, and occasionally IgG globulin, may also exist in multimeric form in serum. In the case of multimer, each of the four units may be the same or different.

Various exemplary assembled immunoadhesins within the scope herein are schematically diagrammed below:

(a) AC_{L}-AC_{L};

(b) AC_{H}-(AC_{H}, AC_{L}-AC_{H}, AC_{L}-V_{H}C_{H}, or V_{L}C_{L}-AC_{H});

(c) AC_{L}-AC_{H}-(AC_{L}-AC_{H}, AC_{L}-V_{H}C_{H}, V_{L}C_{L}-AC_{H}, or V_{L}C_{L}-V_{H}C_{H})

(d) AC_{L}-V_{H}C_{H}-(AC_{H}, or AC_{L}-V_{H}C_{H}, or V_{L}C_{L}-AC_{H});

(e) V_{L}C_{L}-AC_{H}-(AC_{L}-V_{H}C_{H}, or V_{L}C_{L}-AC_{H}); and

(f) (A-Y)ₙ-(V_{L}C_{L}-V_{H}C_{H})₂,

wherein each A represents identical or different adhesin amino acid sequences;

V_{L} is an immunoglobulin light chain variable domain;

V_{H} is an immunoglobulin heavy chain variable domain;

C_{L} is an immunoglobulin light chain constant domain;

C_{H} is an immunoglobulin heavy chain constant domain;

n is an integer greater than 1;

Y designates the residue of a covalent cross-linking agent.

In the interests of brevity, the foregoing structures only show key features; they do not indicate joining (J) or other domains of the immunoglobulins, nor are disulfide bonds shown. However, where such domains are required for binding activity, they shall be constructed to be present in the ordinary locations which they occupy in the immunoglobulin molecules.

Alternatively, the adhesin sequences can be inserted between immunoglobulin heavy chain and light chain sequences, such that an immunoglobulin comprising a chimeric heavy chain is obtained. In this embodiment, the adhesin sequences are fused to the 3′ end of an immunoglobulin heavy chain in each arm of an immunoglobulin, either between the hinge and the C_{H}2 domain, or between the C_{H}2 and C_{H}3 domains. Similar constructs have been reported by Hoogenboom et al., Mol. Immunol., 28:1027-1037 (1991).

Although the presence of an immunoglobulin light chain is not required in the immunoadhesins of the present invention, an immunoglobulin light chain might be present either covalently associated to an adhesin-immunoglobulin heavy chain fusion polypeptide, or directly fused to the adhesin. In the former case, DNA encoding an immunoglobulin light chain is typically coexpressed with the DNA encoding the adhesin-immunoglobulin heavy chain fusion protein. Upon secretion, the hybrid heavy chain and the light chain will be covalently associated to provide an immunoglobulin-like structure comprising two disulfide-linked immunoglobulin heavy chain-light chain pairs. Methods suitable for the preparation of such structures are, for example, disclosed in U.S. Pat. No. 4,816,567, issued 28 Mar. 1989.

Immunoadhesins are most conveniently constructed by fusing the cDNA sequence encoding the adhesin portion in-frame to an immunoglobulin cDNA sequence. However, fusion to genomic immunoglobulin fragments can also be used (see, e.g. Aruffo et al., Cell, 61:1303-1313 (1990); and Stamenkovic et al., Cell, 66:1133-1144 (1991)). The latter type of fusion requires the presence of Ig regulatory sequences for expression. cDNAs encoding IgG heavy-chain constant regions can be isolated based on published sequences from cDNA libraries derived from spleen or peripheral blood lymphocytes, by hybridization or by polymerase chain reaction (PCR) techniques. The cDNAs encoding the “adhesin” and the immunoglobulin parts of the immunoadhesin are inserted in tandem into a plasmid vector that directs efficient expression in the chosen host cells.

In another embodiment, the WISP-1 or WISP-1 antagonist may be covalently modified by linking the polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337, or other like molecules such as polyglutamate. Such pegylated forms may be prepared using techniques known in the art.

Leucine zipper forms of these molecules are also contemplated by the invention. “Leucine zipper” is a term in the art used to refer to a leucine rich sequence that enhances, promotes, or drives dimerization or trimerization of its fusion partner (e.g., the sequence or molecule to which the leucine zipper is fused or linked to). Various leucine zipper polypeptides have been described in the art. See, e.g., Landschulz et al., Science, 240:1759 (1988); U.S. Pat. No. 5,716,805; WO 94/10308; Hoppe et al., FEBS Letters, 344:1991 (1994); Maniatis et al., Nature, 341:24 (1989). Those skilled in the art will appreciate that a leucine zipper sequence may be fused at either the 5′ or 3′ end of the WISP-1 or WISP-1 antagonist molecule.

The WISP-1 polypeptides of the present invention may also be modified in a way to form chimeric molecules by fusing the polypeptide to another, heterologous polypeptide or amino acid sequence. Preferably, such heterologous polypeptide or amino acid sequence is one which acts to oligimerize the chimeric molecule. In one embodiment, such a chimeric molecule comprises a fusion of the WISP-1 polypeptide with a tag polypeptide which provides an epitope to which an anti-tag antibody can selectively bind. The epitope tag is generally placed at the amino- or carboxyl-terminus of the polypeptide. The presence of such epitope-tagged forms of the polypeptide can be detected using an antibody against the tag polypeptide. Also, provision of the epitope tag enables the polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

It is contemplated that anti-WISP-1 antibodies may also be employed in the presently disclosed methods. The anti-WISP-1 may be monoclonal antibodies.

Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include a WISP-1 polypeptide or a fusion protein thereof, such as a WISP-1-IgG fusion protein. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against WISP-1. Preferably, the binding specificity of monoclonal antibodies produced by the hybridoma cells is determined-by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium or RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences, Morrison, et al., Proc. Nat. Acad. Sci. 81, 6851 (1984), or by covalently joining to the immunoglobulin coding sequence all or part of the coding sequence for a non-immunoglobulin polypeptide.

Typically such non-immunoglobulin polypeptides are substituted for the constant domains of an antibody of the invention, or they are substituted for the variable domains of one antigen-combining site of an antibody of the invention to create a chimeric bivalent antibody comprising one antigen-combining site having specificity for WISP-1 and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate.

Single chain Fv fragments may also be produced, such as described in Iliades et al., FEBS Letters, 409:437-441 (1997). Coupling of such single chain fragments using various linkers is described in Kortt et al., Protein Engineering, 10:423-433 (1997). A variety of techniques for the recombinant production and manipulation of antibodies are well known in. the art. Illustrative examples of such techniques that are typically utilized by skilled artisans are described in greater detail below.

(i) Humanized Antibodies

Generally, a humanized antibody has one or more amino acid residues introduced into it from a non-human source. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody.

Accordingly, such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

(ii) Human Antibodies

Human monoclonal antibodies can be made by the hybridoma method. Human myeloma and mouse-human heteromyeloma cell lines for the production of human monoclonal antibodies have been described, for example, by Kozbor, J. Immunol. 133, 3001 (1984), and Brodeur, et al., Monoclonal Antibody Production Techniques and Applications, pp. 51-63 (Marcel Dekker, Inc., New York, 1987).

It is now possible to produce transgenic animals (e.g. mice) that are capable, upon immunization, of producing a repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g. Jakobovits et al., Proc. Natl. Acad. Sci. USA 90, 2551-255 (1993); Jakobovits et al., Nature 362, 255-258 (1993).

Mendez et al. (Nature Genetics 15: 146-156 [1997]) have further improved the technology and have generated a line of transgenic mice designated as “Xenomouse II” that, when challenged with an antigen, generates high affinity fully human antibodies. This was achieved by germ-line integration of megabase human heavy chain and light chain loci into mice with deletion into endogenous J_{H} segment as described above. The Xenomouse II harbors 1,020 kb of human heavy chain locus containing approximately 66 V_{H} genes, complete D_{H} and J_{H} regions and three different constant regions (μ, δ and χ), and also harbors 800 kb of human κ locus containing 32 Vκ genes, Jκ segments and Cκ genes. The antibodies produced in these mice closely resemble that seen in humans in all respects, including gene rearrangement, assembly, and repertoire. The human antibodies are preferentially expressed over endogenous antibodies due to deletion in endogenous J_{H} segment that prevents gene rearrangement in the murine locus.

Alternatively, the phage display technology (McCafferty et al., Nature 348, 552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimicks some of the properties of the B-cell. Phage display can be performed in a variety of formats; for their review see, e.g. Johnson, Kevin S. and Chiswell, David J., Current Opinion in Structural Biology 3, 564-571 (1993). Several sources of V-gene segments can be used for phage display. Clackson et al., Nature 352, 624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222, 581-597 (1991), or Griffith et al., EMBO J. 12, 725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technol. 10, 779-783 [1992]). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the nM range. A strategy for making very large phage antibody repertoires (also known as “the mother-of-all libraries”) has been described by Waterhouse et al., Nucl. Acids Res. 21, 2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e. the epitope governs (imprints) the choice of partner. When the process is repeated in order to replace the remaining rodent V domain, a human antibody is obtained (see PCT patent application WO 93/06213, published 1 Apr. 1993). Unlike traditional humanization of rodent antibodies by CDR grafting, this technique provides completely human antibodies, which have no framework or CDR residues of rodent origin.

As discussed below, the antibodies of the invention may optionally comprise monomeric antibodies, dimeric antibodies, as well as multivalent forms of antibodies. Those skilled in the art may construct such dimers or multivalent forms by techniques known in the art. Methods for preparing monovalent antibodies are also well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

(iii) Bispecific Antibodies

Bispecific antibodies are monoclonal, preferably human or humanized, antibodies that have binding specificities for at least two different antigens. In the present case, one of the binding specificities is for WISP-1. For example, bispecific antibodies specifically binding WISP-1 or WISP-1 variants and another CNN family member (e.g., WISP-2, WISP-3, CTGF, Cyr61, or Nov) or other molecules such as CD44 are within the scope of the present invention.

Methods for making bispecific antibodies are known in the art. Traditionally, the recombinant production of bispecific antibodies is based on the coexpression of two immunoglobulin heavy chain-light chain pairs, where the two heavy chains have different specificities (Millstein and Cuello, Nature 305, 537-539 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in PCT application publication No. WO 93/08829 (published 13 May 1993), and in Traunecker et al., EMBO 10, 3655-3659 (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in PCT Publication No. WO 94/04690, published on Mar. 3, 1994.

For further details of generating bispecific antibodies see, for example, Suresh et al., Methods in Enzymology 121, 210 (1986).

(iv) Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (PCT application publication Nos. WO 91/00360 and WO 92/200373; EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

(v) Antibody Fragments

In certain embodiments, the anti-WISP-1 antibody (including murine, human and humanized antibodies, and antibody variants) is an antibody fragment. Various techniques have been developed for the production of antibody fragments. Traditionally, these fragments were derived via proteolytic digestion of intact antibodies (see, e.g., Morimoto et al., J. Biochem. Biophys. Methods 24:107-117 (1992) and Brennan et al., Science 229:81 (1985)). However, these fragments can now be produced directly by recombinant host cells. For example, Fab′-SH fragments can be directly recovered from E. coli and chemically coupled to form F(ab′)₂ fragments (Carter et al., Bio/Technology 10:163-167 (1992)). In another embodiment, the F(ab′)₂ is formed using the leucine zipper GCN4 to promote assembly of the F(ab′)₂ molecule. According to another approach, Fv, Fab or F(ab′)₂ fragments can be isolated directly from recombinant host cell culture. A variety of techniques for the production of antibody fragments will be apparent to the skilled practitioner. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields an F(ab′)₂ fragment that has two antigen combining sites and is still capable of cross-linking antigen.

The Fab fragments produced in the antibody digestion also contain the constant domains of the light chain and the first constant domain (CH₁) of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

Antibodies are glycosylated at conserved positions in their constant regions (Jefferis and Lund, Chem. Immunol. 65:111-128 [1997]; Wright and Morrison, TibTECH 15:26-32 [1997]). The oligosaccharide side chains of the immunoglobulins affect the protein's function (Boyd et al., Mol. Immunol. 32:1311-1318 [1996]; Wittwe and Howard, Biochem. 29:4175-4180 [1990]), and the intramolecular interaction between portions of the glycoprotein which can affect the conformation and presented three-dimensional surface of the glycoprotein (Hefferis and Lund, supra; Wyss and Wagner, Current Opin. Biotech. 7:409-416 [1996]). Oligosaccharides may also serve to target a given glycoprotein to certain molecules based upon specific recognition structures. For example, it has been reported that in agalactosylated IgG, the oligosaccharide moiety ‘flips’ out of the inter-CH2 space and terminal N-acetylglucosamine residues become available to bind mannose binding protein (Malhotra et al., Nature Med. 1:237-243 [1995]). Removal by glycopeptidase of the oligosaccharides from CAMPATH-1H (a recombinant humanized murine monoclonal IgG1 antibody which recognizes the CDw52 antigen of human lymphocytes) produced in Chinese Hamster Ovary (CHO) cells resulted in a complete reduction in complement mediated lysis (CMCL) (Boyd et al., Mol. Immunol. 32:1311-1318 [1996]), while selective removal of sialic acid residues using neuraminidase resulted in no loss of DMCL. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., Mature Biotech. 17:176-180 [1999]).

Glycosylation variants of antibodies are variants in which the glycosylation pattern of an antibody is altered. By altering is meant deleting one or more carbohydrate moieties found in the antibody, adding one or more carbohydrate moieties to the antibody, changing the composition of glycosylation (glycosylation pattern), the extent of glycosylation, etc. Glycosylation variants may, for example, be prepared by removing, changing and/or adding one or more glycosylation sites in the nucleic acid sequence encoding the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation (including glycosylation pattern) of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, significant variations in the glycosylation pattern of the antibodies can be expected (see, e.g. Hse et al., J. Biol. Chem. 272:9062-9070 [1997]). In addition to the choice of host cells, factors which affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example using endoglycosidase H (Endo H). In addition, the recombinant host cell can be genetically engineered, e.g. make defective in processing certain types of polysaccharides. These and similar techniques are well known in the art.

The glycosylation structure of antibodies can be readily analyzed by conventional techniques of carbohydrate analysis, including lectin chromatography, NMR, Mass spectrometry, HPLC, GPC, monosaccharide compositional analysis, sequential enzymatic digestion, and HPAEC-PAD, which uses high pH anion exchange chromatography to separate oligosaccharides based on charge. Methods for releasing oligosaccharides for analytical purposes are also known, and include, without limitation, enzymatic treatment (commonly performed using peptide-N-glycosidase F/endo-β-galactosidase), elimination using harsh alkaline environment to release mainly O-linked structures, and chemical methods using anhydrous hydrazine to release both N- and O-linked oligosaccharides.

Triabodies are also within the scope of the invention. Such antibodies are described for instance in Iliades et al., supra and Kortt et al., supra.

The antibodies of the present invention may be modified by conjugating the antibody to a cytotoxic agent (like a toxin molecule) or a prodrug-activating enzyme which converts a prodrug (e.g. a peptidyl chemotherapeutic agent, see WO81/01145) to an active anti-cancer drug. See, for example, WO 88/07378 and U.S. Pat. No. 4,975,278. This technology is also referred to as “Antibody Dependent Enzyme Mediated Prodrug Therapy” (ADEPT).

The enzyme component of the immunoconjugate useful for ADEPT includes any enzyme capable of acting on a prodrug in such a way so as to covert it into its more active, cytotoxic form. Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; caspases such as caspase-3; D-alanylcarboxypeptidases, useful for converting prodrugs that contain D-amino acid substituents; carbohydrate-cleaving enzymes such as beta-galactosidase and neuraminidase useful for converting glycosylated prodrugs into free drugs; beta-lactamase useful for converting drugs derivatized with beta-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

The enzymes can be covalently bound to the antibodies by techniques well known in the art such as the use of heterobifunctional crosslinking reagents. Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature, 312: 604-608 (1984).

Further antibody modifications are contemplated. For example, the antibody may be linked to one of a variety of nonproteinaceous polymers, e.g., polyethylene glycol, polypropylene glycol, polyoxyalkylenes, or copolymers of polyethylene glycol and polypropylene glycol, or other molecules such as polyglutamate. The antibody also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Osol, A., Ed., (1980). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

Degenerative cartilagenous disorders contemplated by the invention include Rheumatoid arthritis (RA). RA is a systemic, autoimmune, degenerative disease that can cause symmetrical disruptions in the synovium of both large and small diarthroidal joints. As the disease progresses, symptoms of RA may include fever, weight loss, thinning of the skin, multiorgan involvement, scleritis, corneal ulcers, formation of subcutaneous or subperiosteal nodules and premature death. RA symptoms typically appear during youth, extra-articular manifestations can affect any organ system, and joint destruction is symmetrical and occurs in both large and small joints alike. Extra-articular symptoms can include vasculitis, atrophy of the skin and muscle, subcutaneous nodules, lymphadenopathy, splenomegaly, leukopaenia and chronic anaemia. RA tends to be heterogeneous in nature with a variable disease expression and is associated with the formation of serum rheumatoid factor in 90% of patients sometime during the course of the illness. RA patients typically also have a hyperactive immune system. The majority of people with RA have a genetic susceptibility associated with increased activation of class II major histocompatibility complex molecules on monocytes and macrophages. These histocompatibility complex molecules are involved in the presentation of antigen to activated T cells bearing receptors for these class II molecules. The genetic predisposition to RA is supported by the prevalence of the highly conserved leukocyte antigen DR subtype Dw4, Dw14 and Dw15 in human patients with very severe disease.

Osteoarthritis (OA) is another degenerative cartilagenous disorder that involves a localized disease that affects articular cartilage and bone and results in pain and diminished joint function. OA may be classified into two types: primary and secondary. Primary OA refers to the spectrum of degenerative joint diseases for which no underlying etiology has been determined. Typically, the joint affected by primary OA are the interphalangeal joints of the hands, the first carpometacarpal joints, the hips, the knees, the spine, and some joints in the midfoot. Large joints, such as the ankles, elbows and shoulders tend to be spared in primary OA. In contrast, secondary OA often occurs as a result of defined injury or trauma. Secondary arthritis can also be found in individuals with metabolic diseases such as hemochromatosis and alkaptonuria, developmental abnormalities such as developmental dysplasia of the hips (congenital dislocation of the hips) and limb-length discrepancies, obesity, inflammatory arthritides such as rheumatoid arthritis or gout, septic arthritis, and neuropathic arthritis.

The degradation associated with OA initially appears as fraying and fibrillation of the articular cartilage surface as proteoglycans are lost from the matrix. With continued joint use, surface fibrillation progresses, defects penetrate deeper into the cartilage, and pieces of cartilage tissue are lost. In addition, bone underlying the cartilage (subchondral bone) thickens, and, as cartilage is lost, bone becomes slowly exposed. With asymmetric cartilage destruction, disfigurement can occur. Bony nodules, called osteophytes, often form at the periphery of the cartilage surface and occasionally grow over the adjacent eroded areas. If the surface of these bony outgrowths is permeated, vascular outgrowth may occur and cause the formation of tissue plugs containing fibrocartilage.

Since cartilage is avascular, damage which occurs to the cartilage layer but does not penetrate to the subchondral bone, leaves the job of repair to the resident chondrocytes, which have little intrinsic potential for replication. However, when the subchondral bone is penetrated, its vascular supply allows a triphasic repair process to take place. The suboptimal cartilage which is synthesized in response to this type of damage, termed herein “fibrocartilage” because of its fibrous matrix, has suboptimal biochemical and mechanical properties, and is thus subject to further wear and destruction. In a diseased or damaged joint, increased release of metalloproteinases (MMPs) such as collagenases, gelatinases, stromelysins, aggrecanases, and other proteases, leads to further thinning and loss of cartilage. In vitro studies have shown that cytokines such as IL-1alpha, IL-1beta, TNF-alpha, PDGF, GM-CSF, IFN-gamma, TGF-beta, LIF, IL-2 and IL-6, IL-8 can alter the activity of synovial fibroblast-like cells, macrophage, T cells, and/or osteoclasts, suggesting that these cytokines may regulate cartilage matrix turnover in vivo.

The mechanical properties of cartilage are determined by its biochemical composition. While the collagen architecture contributes to the tensile strength and stiffness of cartilage, the compressibility (or elasticity) is due to its proteoglycan component. In healthy articular cartilage, type II collagen predominates (comprising about 90-95%), however, smaller amounts of types V, VI, IX, and XI collagen are also present. Cartilage proteoglycans (PG) include hydrodynamically large, aggregating PG, with covalently linked sulfated glycosaminoglycans, as well as hydrodynamically smaller nonaggregating PG such as decorin, biglycan and lumican.

Injuries to cartilage may fall into three categories: (1) microdamage or blunt trauma, (2) chondral fractures, and (3) osteochondral fractures.

Microdamage to chondrocytes and cartilage matrix may be caused by a single impact, through repetitive blunt trauma, or with continuous use of a biomechanically unstable joint. Metabolic and biochemical changes such as those found in the early stages of degenerative arthritis can be replicated in animal models involving repetitive loading of articular cartilage. Radin et al., Clin. Orthop. Relat. Res. 131: 288-93 (1978). Such experiments, along with the distinct pattern of cartilage loss found in arthritic joints, highlight the role that biomechanical loading plays in the loss of homeostasis and integrity of articular cartilage in disease. Radin et al., J Orthop Res. 2: 221-234 (1984); Radin et al., Semin Arthritis Rheum (suppl. 2) 21: 12-21 (1991); Wei et al., Acta Orthop Scand 69: 351-357 (1998). While chondrocytes may initially be able to replenish cartilage matrix with proteoglycans at a basal rate, concurrent damage to the collagen network may increase the rate of loss and result in irreversible degeneration. Buckwalter et al., J. Am. Acad. Orthop. Surg. 2: 192-201 (1994).

Chondral fractures are characterized by disruption of the articular surface without violation of the subchondral plate. Chondrocyte necrosis at the injury site occurs, followed by increased mitotic and metabolic activity of the surviving chondrocytes bordering the injury which leads to lining of the clefts of the articular surface with fibrous tissue. The increase in chondrocyte activity is transitory, and the repair response results in insufficient amount and quality of new matrix components.

Osteochondral fractures, the most serious of the three types of injuries, are lesions crossing the tidemark into the underlying subchondral plate. In this type of injury, the presence of subchondral vasculature elicits the three-phase response typically encountered in vascular tissues: (1) necrosis, (2) inflammation, and (3) repair. Initially the lesion fills with blood and clots. The resulting fibrin clot activates an inflammatory response and becomes vascularized repair tissue, and the various cellular components release growth factors and cytokines including transforming growth factor.beta (TGF-beta), platelet-derived growth factor (PDGF), bone morphogenic proteins, and insulin-like growth factors I and II. Buckwalter et al., J. Am. Acad. Orthop. Surg. 2: 191-201 (1994).

The initial repair response associated with osteochondral fractures is characterized by recruitment, proliferation and differentiation of precursors into chondrocytes. Mesenchymal stem cells are deposited in the fibrin network, which eventually becomes a fibrocartilagenous zone. F. Shapiro et al., J. Bone Joint Surg. 75: 532-53 (1993); N. Mitchell and N. Shepard, J. Bone Joint Surg. 58: 230-33 (1976). These stem cells, which are believed to come from the underlying bone marrow rather than the adjacent articular surface, progressively differentiate into chondrocytes. At six to eight weeks after injury, the repair tissue contains chondrocyte-like cells in a matrix of proteoglycans and predominantly type II collagen, with some type I collagen. T. Furukawa et al., J. Bone Joint Surg. 62: 79-89 (1980); J. Cheung et al., Arthritis Rheum. 23: 211-19 (1980); S. O. Hjertquist & R. Lemperg, Calc. Tissue Res. 8: 54-72 (1971). However, this newly deposited matrix degenerates, and the chondroid tissue is replaced by more fibrous tissue and fibrocartilage and a shift in the synthesis of collagen from type II to type I. H. S. Cheung et al., J. Bone Joint Surg. 60: 1076-81 (1978); D. Hamerman, “Prospects for medical intervention in cartilage repair,” Joint cartilage degradation: Basic and clinical aspects, Eds. Woessner J F et al., (1993); Shapiro et al., J. Bone Joint Surg. 75: 532-53 (1993); N. Mitchell & N. Shepard, J. Bone Joint Surg. 58: 230-33 (1976); S. O. Hjertquist & R. Lemperg, Calc. Tissue Res. 8: 54-72 (1971). Early degenerative changes include surface fibrillation, depletion of proteoglycans, chondrocyte cloning and death, and vertical fissuring from the superficial to deep layers. At one year post-injury, the repair tissue is a mixture of fibrocartilage and hyaline cartilage, with a substantial amount of type I collagen, which is not found in appreciable amounts in normal articular cartilage. T. Furukawa, et al., J. Bone Joint Surg. 62: 79-89 (1980).

While inflammation does not appear to be the initiating event in osteoarthritis, inflammation does occur in osteoarthritic joints. The inflammatory cells (i.e. monocytes, macrophages, and neutrophils) which invade the synovial lining after injury and during inflammation produce metalloproteinases as well as catabolic cyokines which can contribute to further release of degradative enzymes. Although inflammation and joint destruction do not show perfect correlation in all animal models of arthritis, agents such as IL-4, IL-10 and IL-13 which inhibit inflammation also decrease cartilage and bone pathology in arthritic animals (reviewed in Martel-Pelletier J. et al. Front. Biosci. 4: d694-703). Application of agents which inhibit inflammatory cytokines may slow OA progression by countering the local synovitis which occurs in OA patients.

OA involves not only the degeneration of articular cartilage leading to eburnation of bone, but also extensive remodelling of subchondral bone resulting in the so-called sclerosis of this tissue. These bony changes are often accompanied by the formation of subchondral cysts as a result of focal resorption. Agents which inhibit bone resorption, i.e. osteoprotegerin or bisphosphonates, have shown promising results in animal models of arthritis. Kong et al. Nature 402: 304-308 (1999).

In systemic lupus erythematosus, the central mediator of disease is the production of auto-reactive antibodies to self proteins/tissues and the subsequent generation of immune-mediated inflammation. These antibodies either directly or indirectly mediate tissue injury. Although T lymphocytes have not been shown to be directly involved in tissue damage, T lymphocytes are required for the development of auto-reactive antibodies. The genesis of the disease is thus T lymphocyte dependent. Multiple organs and systems are affected clinically including kidney, lung, musculoskeletal system, mucocutaneous, eye, central nervous system, cardiovascular system, gastrointestinal tract, bone marrow and blood.

Juvenile chronic arthritis is a chronic idiopathic inflammatory disease which begins often at less than 16 years of age and which has some similarities to RA. Some patients which are rheumatoid factor positive are classified as juvenile rheumatoid arthritis. The disease is sub-classified into three major categories: pauciarticular, polyarticular, and systemic. The arthritis can be severe and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing spondylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. The cell most implicated as key to induction of the disease is the CD8+ T lymphocyte, a cell which targets antigen presented by class I MHC molecules. CD8+ T cells may react against the class I MHC allele HLA-B27 as if it were a foreign peptide expressed by MHC class I molecules. It has been hypothesized that an epitope of HLA-B27 may mimic a bacterial or other microbial antigenic epitope and thus induce a CD8+ T cells response.

The WISP antagonists employed in the invention may be prepared by any suitable method, including recombinant expresssion techniques. Recombinant expression technology is well known to those skilled in the art, and optional materials and methods are described in PCT application, WO 99/21998. Optionally, the WISP antagonists are expressed using host cell such as CHO cells, E. coli or yeast cells. The WISP antagonists may comprise full length polypeptides (defined herein), or variant forms thereof, as well as other modified forms of the WISP polypeptides (such as by fusing or linking to an immunoglobulin, epitope tag, leucine zipper or other non-proteinaceous polymer).

Immunoadhesin molecules are contemplated for use in the methods herein. WISP immunoadhesins may comprise various forms of WISP, such as the full length polypeptide as well as variant or fragment forms thereof. In one embodiment, the molecule may comprise a fusion of the WISP with an immunoglobulin or a particular region of an immunoglobulin. For a bivalent form of the immunoadhesin, such a fusion could be to the Fc region of an IgG molecule. For the production of immunoglobulin fusions, see also U.S. Pat. No. 5,428,130 issued Jun. 27, 1995 and Chamow et al., TIBTECH, 14:52-60 (1996).

In another embodiment, the WISP antagonist may be covalently modified by linking the polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337. Such pegylated forms of the WISP antagonist may be prepared using techniques known in the art.

Leucine zipper forms of these molecules are also contemplated by the invention. “Leucine zipper” is a term in the art used to refer to a leucine rich sequence that enhances, promotes, or drives dimerization or trimerization of its fusion partner (e.g., the sequence or molecule to which the leucine zipper is fused or linked to). Various leucine zipper polypeptides have been described in the art. See, e.g., Landschulz et al., Science, 240:1759 (1988); U.S. Pat. No. 5,716,805; WO 94/10308; Hoppe et al., FEBS Letters, 344:1991 (1994); Maniatis et al., Nature, 341:24 (1989). Those skilled in the art will appreciate that a leucine zipper sequence may be fused at either the 5′ or 3′ end of the WISP polypeptide.

The WISP antagonists of the present invention may also be modified in a way to form chimeric molecules by fusing the antagonist polypeptide to another, heterologous polypeptide or amino acid sequence. Preferably, such heterologous polypeptide or amino acid sequence is one which acts to oligimerize the chimeric molecule. In one embodiment, such a chimeric molecule comprises a fusion of the WISP polypeptide with a tag polypeptide which provides an epitope to which an anti-tag antibody can selectively bind. The epitope tag is generally placed at the amino- or carboxyl-terminus of the polypeptide. The presence of such epitope-tagged forms of the WISP polypeptide can be detected using an antibody against the tag polypeptide. Also, provision of the epitope tag enables the WISP polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

Formulations of WISP antagonists employable with the invention can be prepared by mixing the WISP antagonist having the desired degree of purity with optional pharmaceutically acceptable carriers, excipients or stabilizers (Remington's Pharmaceutical Sciences 16th edition, Osol, A. Ed. [1980]). Such therapeutic formulations can be in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, dextrins, or hyaluronan; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN®, PLURONICS® or polyethylene glycol (PEG).

The WISP antagonists also may be prepared by entrapping in microcapsules prepared, for example by coacervation techniques or by interfacial polymerization, for example, hydroxymethylcellulose or gelatin-microcapsules and poly-(methylmethacrylate) microcapsules, respectively. Such preparations can be administered in colloidal drug delivery systems (for example, liposomes, albumin microspheres, microemulsions, nano-particles and nanocapsules) or in macroemulsions. Such techniques are disclosed in Remington's Pharmaceutical Sciences, 16th Edition (or newer), Osol A. Ed. (1980).

Where sustained-release or extended-release administration of the WISP antagonists is desired in a formulation with release characteristics suitable for the treatment of any disease or disorder requiring administration of such polypeptides, microencapsulation is contemplated. Microencapsulation of recombinant proteins for sustained release has been successfully performed. See, e.g., Johnson et al., Nat. Med. 2: 795-799 (1996); Yasuda, Biomed. Ther. 27: 1221-1223 (1993); Hora et al., Bio/Technology 8: 755-758 (1990); Cleland, “Design and Production of Single Immunization Vaccines Using Polylactide Polyglycolide Microsphere Systems” in Vaccine Design: The Subunit and Adjuvant Approach, Powell and Newman, eds., (Plenum Press: New York, 1995), pp. 439-462; WO 97/03692, WO 96/40072, WO 96/07399 and U.S. Pat. No. 5,654,010.

Suitable examples of sustained-release preparations include semipermeable matrices of solid hydrophobic polymers containing the active molecule, which matrices are in the form of shaped articles, e.g. films, or microcapsules. Examples of sustained-release matrices include one or more polyanhydrides (e.g., U.S. Pat. Nos. 4,891,225; 4,767,628), polyesters such as polyglycolides, polylactides and polylactide-co-glycolides (e.g., U.S. Pat. No. 3,773,919; U.S. Pat. No. 4,767,628; U.S. Pat. No. 4,530,840; Kulkarni et al., Arch. Surg. 93: 839 (1966)), polyamino acids such as polylysine, polymers and copolymers of polyethylene oxide, polyethylene oxide acrylates, polyacrylates, ethylene-vinyl acetates, polyamides, polyurethanes, polyorthoesters, polyacetylnitriles, polyphosphazenes, and polyester hydrogels (for example, poly(2-hydroxyethyl-methacrylate), or poly(vinylalcohol)), cellulose, acyl substituted cellulose acetates, non-degradable polyurethanes, polystyrenes, polyvinyl chloride, polyvinyl fluoride, poly(vinylimidazole), chlorosulphonated polyolefins, polyethylene oxide, copolymers of L-glutamic acid and gamma-ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. Additional non-biodegradable polymers which may be employed are polyethylene, polyvinyl pyrrolidone, ethylene vinylacetate, polyethylene glycol, cellulose acetate butyrate and cellulose acetate propionate.

Alternatively, sustained release formulations may be composed of degradable biological materials. Biodegradable polymers are attractive drug formulations because of their biocompatibility, high responsibility for specific degradation, and ease of incorporating the active drug into the biological matrix. For example, hyaluronic acid (HA) may be crosslinked and used as a swellable polymeric delivery vehicle for biological materials. U.S. Pat. No. 4,957,744; Valle et al., Polym. Mater. Sci. Eng. 62: 731-735 (1991). HA polymer grafted with polyethylene glycol has also been prepared as an improved delivery matrix which reduced both undesired drug leakage and the denaturing associated with long term storage at physiological conditions. Kazuteru, M., J. Controlled Release 59:77-86 (1999). Additional biodegradable polymers which may be used are poly(caprolactone), polyanhydrides, polyamino acids, polyorthoesters, polycyanoacrylates, poly(phosphazines), poly(phosphodiesters), polyesteramides, polydioxanones, polyacetals, polyketals, polycarbonates, polyorthocarbonates, degradable and nontoxic polyurethanes, polyhydroxylbutyrates, polyhydroxyvalerates, polyalkylene oxalates, polyalkylene succinates, poly(malic acid), chitin and chitosan.

Alternatively, biodegradable hydrogels may be used as controlled release delivery vehicles for biological materials and drugs. Through the appropriate choice of macromers, membranes can be produced with a range of permeability, pore sizes and degradation rates suitable for a wide variety of biomolecules.

Alternatively, sustained-release delivery systems for biological materials and drugs can be composed of dispersions. Dispersions may further be classified as either suspensions or emulsions. In the context of delivery vehicles for biological materials, suspensions are a mixture of very small solid particles which are dispersed (more or less uniformly) in a liquid medium. The solid particles of a suspension can range in size from a few nanometers to hundreds of microns, and include microspheres, microcapsules and nanospheres. Emulsions, on the other hand, are a mixture of two or more immiscible liquids held in suspension by small quantities of emulsifiers. Emulsifiers form an interfacial film between the immiscible liquids and are also known as surfactants or detergents. Emulsion formulations can be both oil in water (o/w) wherein water is in a continuous phase while the oil or fat is dispersed, as well as water in oil (w/o), wherein the oil is in a continuous phase while the water is dispersed. One example of a suitable sustained-release formulation is disclosed in WO 97/25563. Additionally, emulsions for use with biological materials include multiple emulsions, microemulsions, microdroplets and liposomes. Microdroplets are unilamellar phospholipid vesicles that consist of a spherical lipid layer with an oil phase inside. E.g., U.S. Pat. No. 4,622,219 and U.S. Pat. No. 4,725,442. Liposomes are phospholipid vesicles prepared by mixing water-insoluble polar lipids with an aqueous solution.

Alternatively, the sustained-release formulations of WISP antagonists may be developed using poly-lactic-coglycolic acid (PLGA), a polymer exhibiting a strong degree of biocompatibility and a wide range of biodegradable properties. The degradation products of PLGA, lactic and glycolic acids, are cleared quickly from the human body. Moreover, the degradability of this polymer can be adjusted from months to years depending on its molecular weight and composition. For further information see Lewis, “Controlled Release of Bioactive Agents from Lactide/Glycolide polymer,” in Biogradable Polymers as Drug Delivery Systems M. Chasin and R. Langeer, editors (Marcel Dekker: New York, 1990), pp. 1-41.

The encapsulated polypeptides or polypeptides in extended-release formulation may be imparted by formulating the polypeptide with a “water-soluble polyvalent metal salts” which are non-toxic at the release concentration and temperature. Exemplary “polyvalent metals” include the following cations: Ca²⁺, Mg²⁺, Zn²⁺, Fe²⁺, Fe³⁺, Cu²⁺, Sn²⁺, Sn⁴⁺, Al²⁺ and Al³⁺, Exemplary anions which form water-soluble salts with the above polyvalent metal cations include those formed by inorganic acids and/or organic acids. Such water-soluble salts have solubility in water (at 20° C.) of at least about 20 mg/ml, alternatively 100 mg/ml, alternatively 200 mg/ml.

Suitable inorganic acids that can be used to form the “water soluble polyvalent metal salts” include hydrochloric, sulfuric, nitric, thiocyanic and phosphoric acid. Suitable organic acids that can be used include aliphatic carboxylic acid and aromatic acids. Aliphatic acids within this definition may be defined as saturated or unsaturated C₂₋₉ carboxylic acids (e.g., aliphatic mono-, di- and tri-carboxylic acids). Commonly employed water soluble polyvalent metal salts which may be used to help stabilize the encapsulated polypeptides of this invention include, for example: (1) the inorganic acid metal salts of halides (e.g., zinc chloride, calcium chloride), sulfates, nitrates, phosphates and thiocyanates; (2) the aliphatic carboxylic acid metal salts calcium acetate, zinc acetate, calcium proprionate, zinc glycolate, calcium lactate, zinc lactate and zinc tartrate; and (3) the aromatic carboxylic acid metal salts of benzoates (e.g., zinc benzoate) and salicylates.

In order for the formulations to be used for in vivo administration, they should be sterile. The formulation may be readily rendered sterile by filtration through sterile filtration membranes, prior to or following lyophilization and reconstitution. The therapeutic compositions herein generally are placed into a container having a sterile access port, for example, an intravenous solution bag or vial having a stopper pierceable by a hypodermic injection needle.

For treatment of the mammal in vivo, the route of administration is in accordance with known methods, e.g., injection or infusion by intravenous, intraperitoneal, intramuscular, intraarterial, intralesional or intraarticular routes, topical administration, by sustained release or extended-release means. Optionally the active compound or formulation is injected directly or locally into the afflicted cartilagenous region or articular joint. The treatment contemplated by the invention may also take the form of gene therapy.

Dosages and desired drug concentrations of pharmaceutical compositions employable with the present invention may vary depending on the particular use envisioned. The determination of the appropriate dosage or route of administration is well within the skill of an ordinary physician. Animal experiments can provide reliable guidance for the determination of effective doses for human therapy. Interspecies scaling of effective doses can be performed following the principles laid down by Mordenti, J. and Chappell, W. “The use of interspecies scaling in toxicokinetics” in Toxicokinetics and New Drug Development, Yacobi et al., Eds., Pergamon Press, New York 1989, pp. 42-96.

When in vivo administration of WISP antagonists are employed, normal dosage amounts may vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344 or 5,225,212. It is anticipated that different formulations will be effective for different treatments and different disorders, and that administration intended to treat a specific organ or tissue, may necessitate delivery in a manner different from that to another organ or tissue.

The formulations used herein may also contain more than one active compound as necessary for the particular indication being treated, preferably those with complementary activities that do not adversely affect each other. The WISP antagonist may be administered in combination with a cytotoxic agent, cytokine or growth inhibitory agent. Such molecules are present in combinations and amounts that are effective for the intended purpose. It may be desirable to also administer antibodies against other immune disease associated or tumor associated antigens, such as antibodies which bind to CD20, CD11a, CD 40, CD18, ErbB2, EGFR, ErbB3, ErbB4, or vascular endothelial growth factor (VEGF). Alternatively, or in addition, two or more antibodies binding the same or two or more different antigens disclosed herein may be coadministered to the patient. Sometimes, it may be beneficial to also administer one or more cytokines to the patient. In one embodiment, the polypeptides of the invention are coadministered with a growth inhibitory agent. For example, the growth inhibitory agent may be administered first, followed by a WISP antagonist of the invention. Still other agents may be administered in combination with WISP antagonist, such as agents like decorin, biglycan, dermatan sulfate or heparin. Simultaneous administration or sequential administration is also contemplated.

The present method may also be administered in combination with any standard cartilage surgical technique. Standard surgical techniques are surgical procedures which are commonly employed for therapeutic manipulations of cartilage, including: cartilage shaving, abrasion chondroplasty, laser repair, debridement, chondroplasty, microfracture with or without subchondral bone penetration, mosaicplasty, cartilage cell allografts, stem cell autografts, costal cartilage grafts, chemical stimulation, electrical stimulation, perichondral autografts, periosteal autografts, cartilage scaffolds, shell (osteoarticular) autografts or allografts, or osteotomy. These techniques are described and discussed in greater detail in Frenkel et al., Front. Bioscience 4: d671-685 (1999).

In an optional embodiment, the WISP antagonists are used in combination with microfracture surgery. Microfracture surgery techniques are known in the art and generally entail surgical drilling into the mammal's bone marrow cavity. Fibrin clots then form, filling the defect in the mammals's body. Subsequently, fibrocartilage forms.

It is contemplated that WISP antagonists can be employed to treat cartilage or chondrocyte cells ex vivo. Such ex vivo treatment may be useful in transplantation and particularly, autologous transplantation. For instance, treatment of cells or tissue(s) containing such cartilage or chondrocyte cells with WISP antagonist, and optionally, with one or more other therapies, such as described above, can be employed to regenerate cartilage tissue of induce differentiation of precursor chondrocyte cells prior to transplantation in a recipient mammal.

Cells or tissue(s) containing cartilage or chondrocyte cells are first obtained from a donor mammal. The cells or tissue(s) may be obtained surgically and preferably, are obtained aseptically. The cells or tissue(s) are then treated with WISP antagonist, and optionally, with one or more other therapies, such as described above.

The treated cells or tissue(s) can then be infused or transplanted into a recipient mammal. The recipient mammal may be the same individual as the donor mammal or may be another, heterologous mammal.

The progress or effectiveness of the therapies described herein can be readily monitored by conventional techniques and assays known to the skilled practicioner.

The activity or effects of the WISP antagonists described herein on cartilage or chondrocytes can be determined without undue experimentation using various in vitro or in vivo assays. By way of example, several such assays are described below.

In one assay, the synthetic and prophylactic potential of WISP antagonist on intact cartilage can be tested. To this end, proteoglycan (PG) synthesis and breakdown, and nitric oxide release are measured in treated articular cartilage explants. Proteoglycans are the second largest component of the organic material in articular cartilage (Kuettner, K. E. et al., Articular Cartilage Biochemistry, Raven Press, New York, USA (1986), p. 456; Muir, H., Biochem. Soc. Tran. 11: 613-622 (1983); Hardingham, T. E., Biochem. Soc. Trans. 9: 489-497 (1981). Since proteoglycans help determine the physical and chemical properties of cartilage, the decrease in cartilage PGs which occurs during joint degeneration leads to loss of compressive stiffness and elasticity, an increase in hydraulic permeability, increased water content (swelling), and changes in the organization of other extracellular components such as collagens. Thus, PG loss is an early step in the progression of degenerative cartilaginous disorders, one which further perturbs the biomechanical and biochemical stability of the joint. PGs in articular cartilage have been extensively studied because of their likely role in skeletal growth and disease. Mow, V. C., & Ratcliffe, A. Biomaterials 13: 67-97 (1992). Proteoglycan breakdown, which is increased in diseased joints, can be measured by quantitating PGs released into the media by articular cartilage explants using the colorimetric DMMB assay. Farndale and Buttle, Biochem. Biophys. Acta 883: 173-177 (1985). Incorporation of ³⁵S-sulfate into proteoglycans is used to measure proteoglycan synthesis.

The evidence linking interleukin-lalpha, IL-1beta, and degenerative cartilagenous diseases is substantial. For example, high levels of IL-1alpha (Pelletier J P et al., “Cytokines and inflammation in cartilage degradation” in Osteoarthritic Edition of Rheumatic Disease Clinics of North America, Eds. RW Moskowitz, Philadelphia, W.D. Saunders Company, 1993, p. 545-568) and IL-1 receptors (Martel-Pelletier et al., Arthritis Rheum. 35: 530-540 (1992) have been found in diseased joints, and IL-1alpha induces cartilage matrix breakdown and inhibits synthesis of new matrix molecules. Baragi et al., J. Clin. Invest. 96: 2454-60 (1995); Baragi et al., Osteoarthritis Cartilage 5: 275-82 (1997); Evans et al., J. Leukoc. Biol. 64: 55-61 (1998); Evans et al., J. Rheumatol. 24: 2061-63 (1997); Kang et al., Biochem. Soc. Trans. 25: 533-37 (1997); Kang et al., Osteoarthritis Cartilage 5: 139-43 (1997). Because of the association of IL-1alpha with disease, the WISP polypeptide can also be assayed in the presence of IL-1alpha.

The production of nitric oxide (NO) can be induced in cartilage by catabolic cytokines such as IL-1. Palmer, R M J et al., Biochem. Biophys. Res. Commun. 193: 398-405 (1993). NO has also been implicated in the joint destruction which occurs in arthritic conditions. Ashok et al., Curr. Opin. Rheum. 10: 263-268 (1998). Unlike normal (undiseased or uninjured) cartilage, osteoarthritic cartilage produced significant amounts of nitric oxide ex vivo, even in the absence of added stimuli such as interleukin-1 or lipopolysaccharide (LPS). In vivo animal models suggest that inhibition of nitric oxide production reduces progression of arthritis. Pelletier, JP et al., Arthritis Rheum. 7: 1275-86 (1998); van de Loo et al., Arthritis Rheum. 41: 634-46 (1998); Stichtenoth, D. O. and Frolich J. C., Br. J. Rheumatol. 37: 246-57 (1998). In vitro, nitric oxide exerts detrimental effects on chondrocyte function, including inhibition of collagen and proteoglycan synthesis, inhibition of adhesion to the extracellular matrix, and enhancement of cell death (apoptosis). Higher concentrations of nitrite are found in synovial fluid from osteoarthritic patients than in fluid from rheumatoid arthritic patients. Renoux et al., Osteoarthritis Cartilage 4: 175-179 (1996). Furthermore, animal models suggest that inhibition of nitric oxide production reduces progression of arthritis. Pelletier, J. P. et al., Arthritis Rheum. 7: 1275-86 (1998); van de Loo et al., Arthritis Rheum. 41: 634-46 (1998); Stichtenoth, D. O. & Frolich, J. C., Br. J. Rheumatol. 37: 246-57 (1998). Since NO also has effects on other cells, the presence of NO within the articular joint could increase vasodilation and permeability, potentiate cytokine release by leukocytes, and stimulate angiogenic activity. Since NO likely play a role in both the erosive and the inflammatory components of joint diseases, a factor which decreases nitric oxide production would likely be beneficial for the treatment of degenerative cartilagenous disorders.

The assay to measure nitric oxide production is based on the principle that 2,3-diaminonapthalene (DAN) reacts with nitrite under acidic conditions to form 1-(H)-naphthotriazole, a fluorescent product. As NO is quickly metabolized into nitrite (NO₂⁻¹) and nitrate (NO₃⁻¹), detection of nitrite is one means of detecting (albeit undercounting) the actual NO produced by cartilage.

The ability of a WISP antagonist to enhance, promote or maintain the viability of chondrocytes in cultures in the absence of serum or other growth factors can also be examined. Articular chondrocytes are first prepared by removal of the extracellular matrix and cultured in a monolayex, which is believed to approximate the latter stages of cartilage disorders when the matrix has been depleted. The assay is a colorimetric assay that measures the metabolic activity of the cultured cells based on the ability of viable cells to cleave the yellow tetrazolium salt MTT to form purple formazan crystals. This cellular reduction reaction involves the pyridine nucleotide cofactors NADH and NADPH. Berridge, M. V. & Tan, A. S., Arch. Biochem. Biophys. 303: 474 (1993). The solubilized product is spectrophotometrically quantitated on an ELISA reader.

Yet another assay examines the effects of WISP polypeptides on proteoglycan synthesis in patellae (kneecaps) of mice. This assay uses intact cartilage (including the underlying bone) and thus tests factors under conditions which approximate the in vivo environment of cartilage. Compounds are either added to patellae in vitro, or are injected into knee joints in vivo prior to analysis of proteoglycan synthesis in patellae ex vivo. As has been shown previously, in vivo treated patellae show distinct changes in PG synthesis ex vivo (Van den Berg et al., Rheum. Int. 1: 165-9 (1982); Vershure, P. J. et al., Ann. Heum. Dis. 53: 455-460 (1994); and Van de Loo et al., Arthrit. Rheum. 38: 164-172 (1995). In this model, the contralateral joint of each animal can be used as a control.

A guinea pig model can be employed to measure the effects of WISP polypeptides on both the stimulation of PG synthesis and inhibition of PG release in articular cartilage explants from a strain of guinea pigs, Dunkin Hartley (DH), which spontaneously develops knee osteoarthritis (OA). Most other animal models which cause rapidly progressing joint breakdown resemble secondary OA more than the slowly evolving human primary OA. In contrast, DH guinea pigs have naturally occurring slowly progressive, non-inflammatory OA-like changes. Because the highly reproducible pattern of cartilage breakdown in these guinea pigs is similar to that seen in the human disorder, the DH guinea pig is a well-accepted animal model for osteoarthritis. Young et al., “Osteoarthritis”, Spontaneous animal models of human disease vol. 2, pp. 257-261, Acad. Press, New York. (1979); Bendele et al., Arthritis Rheum. 34: 1180-1184; Bendele et al., Arthritis Rheum. 31: 561-565 (1988); Jimenez et al., Laboratory Animal Sciences 47 (6): 598-601 (1997); Wei et al., Acta Orthop Scand 69: 351-357 (1998)). Initially, these animals develop a mild OA that is detectable by the presence of minimal histologic changes. However, the disease progresses, and by 16-18 months of age, moderate to severe cartilage degeneration within the joints is observed. As a result, the effect of the WISP polypeptide on the cartilage matrix of the DH guinea pigs over the progression of the disease would be indicative of the therapeutic effect of the compound in the treatment of OA at different stages of joint destruction.

The metabolic changes associated with diabetes mellitus (diabetes) affect may other organ and musculo-skeletal systems of the afflicted organism. For example, in humans, the incidence of musculoskeletal injuries and disorders is increased with the onset of diabetes, and diabetes is considered a risk factor for the development of arthritis.

A syndrome similar to diabetes can be induced in animals by administration of streptozotocin (STZ). Portha B. et al., Diabete Metab. 15: 61-75 (1989). By killing pancreatic cells which produce insulin, STZ decreases the amount of serum insulin in treated animals. STZ-induced diabetes is associated with atrophy and depressed collagen content of connective tissues including skin, bone and cartilage. Craig, R. G. et al., Biochim. Biophys. Acta 1402: 250-260 (1998). In this assay, the patellae of treated STZ-treated mice are incubated in the presence of the WISP polypeptide and the resulting matrix synthesis is analyzed. The ability of the WISP polypeptide to increase or restore the level of PG synthesis to that of untreated controls is indicative of the therapeutic potential.

In another embodiment of the invention, kits and articles of manufacture containing materials useful for the diagnosis or treatment of the disorders described above are provided. The article of manufacture comprises a container and an instruction. Suitable containers include, for example, bottles, vials, syringes, and test tubes. The containers may be formed from a variety of materials such as glass or plastic. The container holds a composition which is effective for diagnosing or treating the degenerative cartilagenous disorder, and may have a sterile access port (for example the container may be an intravenous solution bag or a vial having a stopper pierceable by a hypodermic injection needle). The active agent in the composition will typically be a WISP antagonist. The composition can comprise any or multiple ingredients disclosed herein. The instruction on, or associated with, the container indicates that the composition is used for diagnosing or treating the condition of choice. For example, the instruction could indicate that the composition is effective for the treatment of osteoarthritis arthritis, rheumatoid arthritis or any other degenerative cartilagenous disorder. The article of manufacture may further comprise a second container comprising a pharmaceutically-acceptable buffer, such as phosphate-buffered saline, Ringer's solution and dextrose solution. Alternatively, the composition may contain any of the carriers, excipients and/or stabilizers mentioned herein. It may further include other materials desirable from a commercial and user standpoint, including other buffers, diluents, filters, needles, syringes, and package inserts with instructions for use.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va. Unless otherwise noted, the present invention uses standard procedures of recombinant DNA technology, such as those described hereinabove and in the following textbooks: Sambrook et al., Molecular Cloning: A Laboratory Manual, Cold Spring Harbor Press N.Y., 1989; Ausubel et al., Current Protocols in Molecular Biology, Green Publishing Associates and Wiley Interscience, N.Y., 1989; Innis et al., PCR Protocols: A Guide to Methods and Applications, Academic Press, Inc., N.Y., 1990; Harlow et al., Antibodies: A Laboratory Manual, Cold Spring Harbor Press, Cold Spring Harbor, 1988; Gait, M. J., Oligonucleotide Synthesis, IRL Press, Oxford, 1984; R. I. Freshney, Animal Cell Culture, 1987; Coligan et al., Current Protocols in Immunology, 1991.

In the assays described below, the following methods and materials were employed:

Materials:

Full length murine WISP-1 (Pennica et al., Proc. Natl. Acad. Sci., 95:14717-14722 (1998); WO 99/21998) was cloned into an expression vector encoding the human IgG1 Fc region downstream of the WISP-1 sequence as described previously for TNFR1 (Ashkenazi et al., Proc. Natl. Acad. Sci., 88:10535-10539 (1991)). The resulting recombinant fusion protein (WISP-1-Fc) was synthesized in a baculovirus expression system using Sf9 insect cells and purified to homogeneity from serum-free conditioned medium by affinity chromatography on a protein A-Sepharose Fast Flow (Pharmacia Biotech, Sweden) column. Unadsorbed proteins were washed out with 50 mM sodium phosphate buffer containing 1 M NaCl. WISP-1-Fc was eluted with 100 mM glycine pH 2.5 and the pH was neutralized with 0.1 volume of 3M Tris-HCl pH 8. After dialysis (20 mM Tris-HCl, pH 7.5, 150 mM) the purified protein was concentrated by ultrafiltration using Centriprep-30 (Millipore Corp., Bedford, Mass.) and the purity estimated by SDS-PAGE and silver staining.

Fatty acid ultra free bovine serum albumin (BSA) fraction V and the complete EDTA-free protease inhibitor cocktail tablets were from Roche Molecular Biochemicals (Indianapolis, Ind.). The biotinylated horse anti-mouse IgG was purchased from Jackson ImmunoResearch Laboratories (West Grove, Pa.). FITC conjugated streptavidin and Hoechst 33342 were from Molecular Probes (Eugene, Oreg.). The Renaissance TSA indirect amplification kit was bought from NEN Life Science Products (Boston, Mass.). Vectashield mounting media was obtained from Vector (Burlingame, Calif.) and the Tissue-Tek OCT compound was from Miles (Elkhart,. Ind.). Collagenase type 2, bovine insulin, transferrin and sodium selenite were purchased from Sigma (St-Louis, Mo.). Recombinant human BMP-2 was purchased from R & D Systems (Minneapolis, Minn.) and recombinant human GDF-5 from Antigenix America Inc. (Huntington, N.Y.). WISP-1 monoclonal antibody was generated as previously described. Desnoyers et al., J Biol Chem, 276: 47599-47607 (2001).

In Situ Hybridization

Localization of gene expression was executed as described previously (Holcomb et al., Embo J, 19: 4046-4055 (2000) using ³³P-labeled sense and antisense riboprobes transcribed from a 740 bp PCR product corresponding to nucleotides 440-1180 of mouse WISP-1 (NM_{—}018865).

Immunofluorescence

Sections (10 μm) of OCT embedded rat E18 embryos were washed with PBS and the non-specific binding sites were blocked for 20 minutes in PBS/ 3% BSA containing 1.5% normal horse serum. Avidin and biotin binding sites were blocked with the avidin/biotin blocking kit from Vector (Burlingame, Calif.) and the slides were incubated with 1 pg/ml mouse monoclonal anti-WISP-1 antibody (clone 9C10) in PBS/3% BSA containing 1.5% normal horse serum for 1 hour, washed and fixed in PBS/4% paraformaldehyde for 10 minutes. The sections were washed and incubated for 30 minutes with 1:200 biotinylated horse anti-mouse IgG in HBS-C/3% BSA. The slides were washed, fixed and the signal amplified using the TSA indirect amplification kit according to the manufacturer instructions. The slides were incubated for 30 minutes with streptavidin conjugated FITC (1:1000). The sections were washed, mounted in Vectashield mounting media containing 1 μg/ml Hoechst 33342 and visualized under a Nikon Eclipse 800 fluorescent microscope.

In Situ Ligand Binding

Binding of WISP-1-Fc to rat embryo sections was evaluated using the in situ ligand binding procedure previously described Desnoyers et al., J Biol Chem, 276: 47599-47607 (2001); Desnoyers et al., J Histochem Cytochem, 49: 1509-1518 (2001). No signal was detected when WISP-1-Fc was omitted or the anti human IgG antibody replaced by an irrelevant antibody (anti-gp 120). The binding pattern described for WISP-1-Fc was unique and different from the binding pattern observed for a control protein (human IgG).

Primary Porcine Chondrocytes Isolation

The metacarpo-phalangeal joint of 4-6 month old female pigs was aseptically opened, and articular cartilage was dissected free of the underlying bone. The cartilage was pooled, minced, washed and and digested overnight at 37° C. with collagenase. The digest was filtered through a 50 μm sieve and the cells were washed, seeded at 25,000 cell/cm² in Ham-F12 containing 10% FBS and 4 μg/ml gentamycin and maintained at 37° C. under 5% CO². Cells were fed every 3 days and reseeded every 5 days. After 11 days in culture, 50-60% of the primary chondrocytes had lost their chondrocytic character and reverted to a mesenchymal phenotype characterized by a spindloid bipolar shape and a switch from collagen 2 to collagen 1 expression.

Cell Binding

Binding of WISP-1-Fc to dedifferentiated porcine primary chondrocytes was executed as previously described (Desnoyers et al., J Histochem Cytochem, 49: 1509-1518 (2001). No signal was detected when WISP-1-Fc was omitted or the anti human IgG antibody replaced by an irrelevant antibody (anti-gp 120).

Cell Culture

Normal human dermal fibroblasts (NHDF) and normal human lung fibroblasts (NHLF) were purchased from Cambrex (Walkersville, Md.). C57MG mouse mammary epithelial cell line was given by Dr. Diane Pennica (Genentech, Calif.). NIH/3T3 mouse fibroblasts, MC3T3-E1 clone 14 mouse calvaria preosteoblasts, and the mouse C2C12 skeletal muscle myoblasts were purchased from American Type Culture Collection (Manassas, Va.). ST2 mouse bone marrow stromal cells and ATDC5 mouse embryonal carcinoma-derived chondrogenic cell line were purchased from RIKEN (Tsukuba, Japan).

MC3T3-E1 cells were maintained in a mixture (1:1) of DME and Ham F-12 (DME/F12) medium supplemented with 10% FBS until they reached confluency. Osteoblastic differentiation was induced as previously described (Wang et al. J Bone Miner Res, 14: 893-903 (1999). Briefly, cells were grown to confluency in α-modified Eagle's medium containing 10% FBS and treated with 50 μg/ml ascorbic acid. The inorganic phosphate concentration was raised to 3 mM and the cells were treated an additional 2 days. ST2 cells were maintained in RPMI-1640 containing 10% FBS and C2C12 cells in DME/F12 medium supplemented with 15% FBS. To induce osteoblastic differentiation, cells were grown to confluency and treated with 300 ng/ml BMP-2 (Katagiri et al. J Cell Biol, 127: 1755-1766 (1994); Gong et al. Cell, 107: 513-523 (2001).

ATDC5 cells were maintained in DME/F12 medium supplemented with 5% FBS, 10 μg/ml bovine insulin, 10 μg/ml human transferrin and 30 nM sodium selenite. ATDC5 cells expressing high level of WISP-1 (ATDC5/WISP-1H) or lower level of WISP-1 (ATDC5/WISP-1L) were generated by cotransfecting human WISP-1 in a pRK vector with pSVi puromycin plasmid using Fugene6 according to the manufacturer's instructions (Roche). After 48 hours, cells were selected in media containing 2 μg/ml puromycin. After 2 weeks, clones were isolated and WISP-1 expression was evaluated by immunofluorescence. Control cell lines were generated using the same procedure following the transfection of the empty pRK vector. Chondrocytic differentiation was induced by treating ATDC5 cells with BMP-2 or GDF-5 as previously described (Nakamura et al. Exp Cell Res, 250: 351-363 (1999).

ATDC5 cell proliferation was measured by seeding 10⁴ cells in 10 cm2 petri dishes in culture media supplemented with 0.5% FBS. At indicated time points, the viable cells were counted using a hemacytometer after trypsinization.

Immunoprecipitation and Western Blot Analysis

Stably transfected ATDC5 cells (2×10⁶) were cultured overnight in 4 ml of 1:1 Ham's F-12:DMEM media. A specific monoclonal antibody (Desnoyers et al. J Biol Chem, 276: 47599-47607 (2001) was used to immunoprecipitate WISP-1 from culture media and lysates using a previously described protocol (Tice et al. J Biol Chem, 277: 14329-14335 (2002). The immunoprecipitate was electrophoresed on SDS-PAGE (BIO-RAD) and electrotransferred to PVDF membrane (BIO-RAD). WISP-1 was immunodetected with a biotinylated monoclonal antibody and visualized with the West Femto chemiluminescent substrate (Pierce). An equivalent of 0.5×10⁶ cells/lane and 0.2×10⁶ cells/lane were analyzed for supernatant and cell lysate respectively.

Real Time RT-PCR Analysis

Total RNA was extracted from cells with Tri-Reagent (Molecular Research Center, Cincinnati, Ohio). Specific primers and fluorogenic probes were used to amplify and quantitate gene expression (Winer et al. Anal Biochem, 270: 41-49 (1999). The gene specific signals were normalized to the glyceraldehyde-3-phosphate dehydrogenase housekeeping gene. Triplicate sets of data were averaged for each condition. All TaqMan RT-PCR reagents were purchased from Applied Biosystems (Foster City, Calif.).

Alkaline Phosphatase Assay

Cells were washed twice with PBS and lysed with 20 mM Tris, pH 7.4, 150 mM NaCl, 1% Triton X-100 for 5 minutes on ice. Twenty microliters of the lysate was added to 80 pl of Attophos substrate (Roche) and incubated for 5 minutes at room temperature. The fluorescence was measured (excitation, 420 nm ; emission, 560 nm) and the alkaline phosphatase activity was determined by comparison to a standard curve of enzymatic product. Cell lysates were analyzed for protein content using the micro-BCA Assay kit (Pierce), and alkaline phosphatase activity was normalized for total protein concentration.

Mouse Femoral Fracture Healing Model

A midshaft, fixed femur fracture was created in anesthetized 6 to 8 weeks old male C57BL6 mice (Charles River Laboratories) following a previously described procedure (Bonnarens and Einhorn, J Orthop Res, 2: 97-101 (1984). All animal experimentation was conducted in accordance with National Guidelines.

Tissue Distribution of WISP-1

In situ hybridization (ISH) was performed to elucidate the spatiotemporal profile of WISP-1 expression during embryonic skeletogenesis. At E10.5, before ossification begins, WISP-1 was weakly expressed in the perichondrial mesenchyme from cartilage primordium of developing endochondral bones (data not shown). As skeletal development progressed, WISP-1 expression increased in the mesenchymal cell layer. surrounding the cartilage anlagen. At E12.5 WISP-1 expression was found in osteoblasts of bones undergoing endochondral or intramembranous ossification (FIG. 1A). Some expression was also found in the myocardium and subcutaneous mesoderm (data not shown). At E15.5, WISP-1 expression was high in osteoblasts and associated periosteal cells of vertebrae, ribs and along the diaphysis forming the cortex of the long bone after ossification has begun. WISP-1 expression was more prominent at sites of intramembranous ossification (FIG. 1D). The signal was predominant in osteoblasts and periosteal cells of the developing calvarium and maxilla. WISP-1 was low or undetectable in chondrocytes and other cells surrounding osteogenic cells.

The presence of WISP-1 protein at sites of developing bone was assessed by immunofluorescence in E18 rat embryos. An intense fluorescent staining pattern was observed that closely matched the ISH expression profile (FIG. 2). WISP-1 was found in osteoblasts at all sites of endochondral and intramembranous ossification. The staining was intense in osteoblasts lining the developing calvaria, mandible, clavicle, vertebrae and ribs. No staining was observed in the perichondrium and chondroblasts.

WISP-1 is Expressed by Differentiating Osteoblasts

WISP-1 expression was measured in various cell types (FIG. 3A). Although absent in primary human normal lung and skin fibroblasts, C57MG mammary epithelial cells or ATDC5 chondrogenic cells, WISP-1 was expressed in NIH3T3 fibroblast cells and C2C12 skeletal muscle progenitor cells. Higher levels of WISP-1 expression were found in MC3T3-E1 calvaria preosteoblasts and ST2 osteoblastic bone marrow stromal cells.

WISP-1 expression was monitored during osteoblast differentiation using the MC3T3-E1 and ST2 osteogenic cell lines (Wang et al. J Bone Miner Res 14: 893-903 (1999); Gong et al. Cell, 107: 513-523 (2001). When placed in differentiating medium, these cells progressively adopted an osteoblast phenotype as demonstrated by their increase in osteocalcin expression and alkaline phosphatase activity (FIG. 3). In these cells, the level of WISP-1 expression did not change during the osteoblastic differentiation and remained elevated at all time. Because WISP-1 is expressed in preosteoblastic cells, it could represent an early event that precedes the commitment of MC3T3-E1 and ST2 cells to the osteoblastic lineage. To test this, WISP-1 expression was measured in an osteoblastic transdifferentiation model using the C2C12 skeletal muscle progenitor cells (Katagiri et al. J Cell Biol, 127: 1755-66 (1994). In these cells WISP-1 expression rapidly increased upon induction of the osteogenic transdifferentiation with BMP-2 (FIG. 3H). These results suggest that WISP-1 is predominantly expressed by cells of the osteoblastic lineage and that its induction occurs early during the acquisition of this phenotype.

WISP-1 Binds to the Perichondrium

To better understand the role of WISP-1 in skeletal development, its in situ binding to sagittal sections of rat embryo was analyzed. At embryonic stage E14, WISP-1 interacted with the perichondrial mesenchyme and the condensing prechondroblastic cells of cartilage primordium (FIG. 4). At stage E18, WISP-1 bound only to mesenchymal cells of the perichondrium and no fluorescence associated to the chondroblasts or chondrocytes was found. No signal was detected when WISP-1 was omitted or replaced by a control protein or when an unrelated antibody was used.

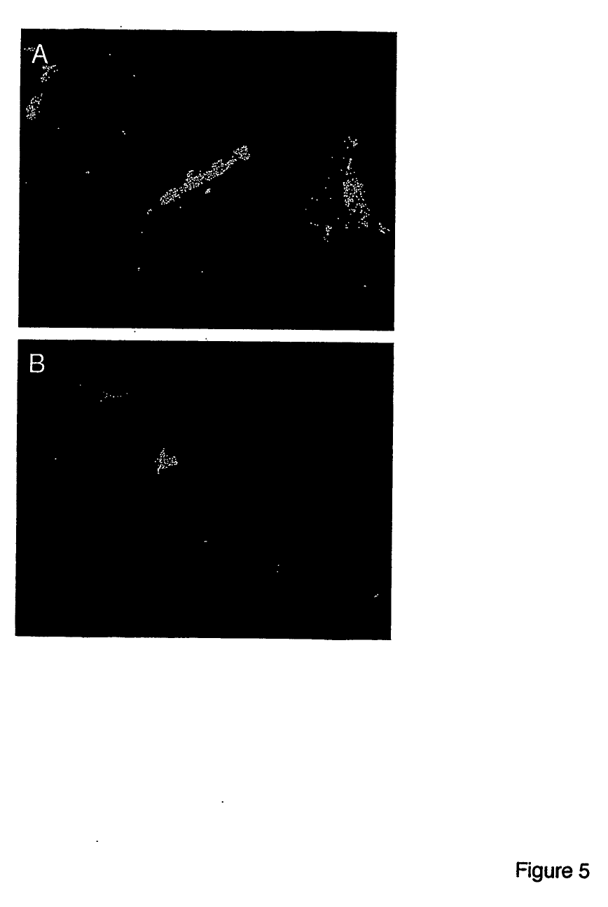

The interaction of WISP-1 with mesenchymal cells was evaluated using primary porcine chondrocytes that had adopted a mesenchymal phenotype after 11 days in culture. WISP-1 binding revealed an irregular pattern associated with patches and points of focal adhesion (FIG. 5a). Intense fluorescent staining was observed at points of contact between adjacent cells (FIG. 5b). WISP-1 interaction with mesenchymal cells could be involved in cell-cell communication.

WISP-1 Acts On Chondrocytic Progenitors

WISP-1 activity on chondrocyte progenitors was investigated by generating ATDC5 chondrogenic cell lines stably transfected with WISP-1. A cell line expressing a high level of WISP-1 (ATDC5/WISP-1H), a cell line expressing a low level of WISP-1 (ATDC5/WISP-1L) and a cell line transfected with an empty vector (ATDC5/Control) were analyzed. Compared to ATDC5/WISP-1L cells, ATDC5/WISP-1H cells had a WISP-1 RNA level 1.8 fold higher (data not shown) and a protein level 2 fold higher (FIG. 6A). When grown to confluency the WISP-1 expressing cell lines demonstrated an increased density compared to the control cell line (FIG. 6C). The saturation density of ATDC5/WISP-1H cell line increased by 1.8 fold and the ATDC5/WISP-1L by 1.6 fold compared to the ATDC5/control cell line (FIG. 6B). No significant differences were found between the density of the ATDC5/control cell line and the parental cell line at confluency (data not shown). The WISP-1 transfectants also demonstrated an increased proliferation compared to the ATDC5/control and the parental cell line. After 11 days, the ATDC5/WISP-1H and the ATDC5/WISP-1L cell population increased by 6 and 2.5 fold respectively compared to the ATDC5/control cell line (FIG. 6D). The growth rate of the ATDC5/contol cell line and the parental cell line were identical.

The differentiation state of the various ATDC5 cell lines was assessed by evaluating their collagen 2 expression level. Before the chondrocytic differentiation was induced, the level of collagen 2 expression was comparable in ATDC5/control and ATDC5/WISP-1L cells but reduced 10 fold in the ATDC5/WISP-1H cells compared to the control cell line (FIG. 6E). The induction of chondrocytic differentiation by BMP-2 or GDF-5, significantly increased collagen 2 expression in ATDC5/control cells. On the other hand, collagen 2 induction was greatly diminished in ATDC5/WISP-1L cells and nearly abolished in ATDC5/WISP-1H cells. These results indicate that WISP-1 increases pre-chondrogenic cells proliferation and saturation density and prevents their progression along the chondrocytic lineage.

WISP-1 Expression is Induced during Bone Fracture Repair.

Because signals regulating embryonic bone formation are recapitulated during fracture repair, WISP-1 temporal expression was evaluated in a mouse model of bone fracture healing (Vortkamp.et al. Mech Dev, 71: 65-76 (1998). WISP-1 signal was prominent at day 3 post-fracture and gradually decreased until day 21 where it could no longer be detected (FIG. 7).

At day 3 and 5 post-fracture, WISP-1 was found in mesenchymal cells within the provisional callus formed along the periosteal surface. Weak expression was also observed in osteoblastic cells lining the periosteum adjacent to the fracture site. At day 7, the osteoblasts along the islands of woven bone within the provisional callus were expressing WISP-1. At day 14 post-fracture, WISP-1 expression was strongest over osteoblasts aggregated along bone spicules bridging islands of woven bone within the hard callus. By day 21, WISP-1 signal was absent from the remodeled bony callus. WISP-1 temporal expression pattern implies a role in early fracture repair that would mirror its function during bone development.

Skeletogenesis involves the commitment of mesenchymal progenitor cells to chondrogenic and osteogenic lineages and their terminal differentiation in chondrocytes or osteoblasts (See, e.g., Karsenty G, Nature, 423: 316-318 (2003); Karsenty and Wagner, Dev Cell, 2: 389-406 (2002). Factors involved in the differentiation process are present in the committed progenitor cells of the appropriate lineage before the terminal differentiation has taken place. During mouse development, WISP-1 expression was initiated at day 10.5 in pluripotent mesenchymal cells surrounding the cartilagenous skeletal templates. WISP-1 expression progressively increased during the mesenchymal condensation of the developing skull and appendicular skeleton and reached a maximum in newly differentiated osteoblasts. By day 15.5, WISP-1 was located in all osteoblasts regardless of their future mode of ossification. Although WISP-1 is expressed early during development, it was never found in mesenchymal cell aggregates that will later differentiate into chondrocytes through the endochondral process. WISP-1 expression was restricted to cells of the osteoblastic lineage at sites of endochondral and intramembrous ossification. Using the skeletal muscle progenitor C2C12 cell line, WISP-1 expression gradually increased in cells induced to transdifferentiate along the osteoblastic lineage. Because WISP-1 expression appears early in lineage specific progenitor cells, it is likely to play a role during the osteoblastic differentiation process.

The in situ ligand binding analysis described above identified the potential site of WISP-1 action to the perichondral mesenchyme of developing bones. WISP-1 interaction with mesenchymal cells was confirmed using cultured dedifferentiated primary chondrocytes. WISP-1 binds to cells of fibroblastic phenotype through its interaction with decorin and biglycan (Desnoyers et al. J Biol Chem, 276: 47599-47607 (2001). Decorin and biglycan are small leucine-rich repeat proteoglycans highly expressed at sites of cartilage and bone formation during development (Wilda et al. J Bone Miner Res, 15: 2187-96 (2000). Their importance in osteogenesis has been demonstrated in null mice models and human diseases (Ameye and Young, Glycobiology, 12: 107R-116R (2002); Chen et al. J Bone Miner Res, 17: 331-340 (2002); Corsi et al. J Bone Miner Res, 17: 1180-1189 (2002). WISP-1 likely bound to the surface of mesenchymal cells of the perichondrium through its interaction with decorin and biglycan. In vivo, WISP-1 secreted by mesenchymal cells of the osteoblastic lineage could bind to decorin and biglycan present in the extracellular matrix (ECM). The concept of a growth factor and cytokine depot has been suggested for the proteoglycans (Iozzo, Proteoglycans: Structure, Biology and Molecular Interactions, 1-4 (2000). This specific interaction would modulate WISP-1 diffusion range, availability and activity. The importance of intercellular communication mediated by extracellular matrix proteins during limb development has been demonstrated (Lonai, J Anat, 202: 43-50 (2003). Consequently, WISP-1 tethered to the ECM could act in a paracrine fashion on neighboring mesenchymal cells committed to the chondrogenic lineage.

In chondrocytic cell lines stably transfected with WISP-1, WISP-1 increased proliferation, saturation density and promoted the expression of genes associated with undedifferentiated mesenchymal cells while repressing genes linked to chondrocyte differentiation. In addition, it attenuated the induction of chondrocytic differentiation by added exogenous growth factors. Taken together, these results suggest that WISP-1 is a negative regulator of chondrocyte differentiation.

Chondrocyte proliferation, commitment and differentiation depends on their local environment, autocrine and paracrine regulation (Quarto et al. Endocrinology, 138: 4966-4976 (1997). Wnt genes were shown to be important paracrine regulators of chondrocyte and osteoblast differentiation during vertebrate skeletal development. Wnt-1, Wnt-5a, Wnt-7a, Wnt-14 negatively regulate chondrogenesis whereas Wnt-4 and Wnt-8 promote chondrocyte maturation (Rudnicki and Brown, Dev Biol, 185: 104-18 (1997); Hartmann and Tabin, Development, 127: 3141-59 (2000); Hartmann and Tabin, Cell, 104: 341-51 (2001); Enomoto-Iwamoto et al. Dev Biol, 251: 142-56 (2002). Wnt signaling also promotes osteoblast differentiation and regulates bone accrual during development (Harada and Rodan, Nature, 423: 349-355 (2003). Wnt regulatory activity requires the integrity of its pathway, suggesting that Wnt/p-catenin target genes are involved in the osteoblastic and chondrocytic differentiation of mesenchymal progenitor cells (Hartmann and Tabin, Development, 127: 3141-59 (2000); Gong et al. Cell, 107: 513-23 (2001). Because WISP-1 is a Wnt/β-catenin downsream gene, it could constitute an effector of the Wnt regulatory cascade acting during skeletogenesis (Pennica et al., Proc Natl Acad Sci U S A, 95: 14717-14722 (1998); Xu et al. Genes Dev, 14: 585-95 (2000).

During endochondral ossification, proliferation and condensation of mesenchymal cells is stopped by their differentiation into hypertrophic chondrocytes. The appropriate size and shape of the bones depends on a balance between proliferation and differentiation of mesenchymal cells forming the cartilage anlagens (Kronenberg, Nature,423:332-6 (2003). In vitro, WISP-1 negatively regulates chondrocytic differentiation. Because it is expressed at sites of endochondral ossification during development, WISP-1 could prevent premature completion of chondrocytic differentiation and insure adequate morphogenesis of the skeletal structure. Alternately, WISP-1 expressed at an early stage during osteoblastic differentiation could contribute to phenotype definition by preventing precursor cells from reverting to a chondrocytic lineage.

Because several pathways regulating embryonic skeletal development are reactivated during bone healing, WISP-1 expression patterns were analyzed during fracture repair (Vortkamp et al., Mech Dev, 71: 65-76 (1998). Bone healing proceeds through three distinct phases, namely inflammation, reparation and remodeling (Bolander, Proc Soc Exp Biol Med, 200: 165-170 (1992); Sandberg et al., Clin Orthop, 289: 292-312 (1993). The first phase begins with the activation of the inflammatory cell response and the recruitment and proliferation of mesenchymal stem cells surrounding the fracture site. During the reparation phase, endochondral and intramembranous bone synthesis takes place. Mesenchymal cells of the subperiostal bone differentiate into chondrocytes to form.the fibrocartilagenous soft callus. Chondrocytes of the soft callus that progressively differentiate into hypertrophic chondrocytes are invaded by blood vessels and osteogenic cells and are ultimately replaced by bone. Also, the periosteal mesenchymal cells adjacent to the injured bone directly differentiate into osteoblasts and start the production of bone matrix to form the hard callus. The formation of primary bone is followed by extensive remodeling until the damaged skeletal element regains original shape and size. During the bone healing process, WISP-1 expression recapitulated the pattern observed during embryonic development.

Soon after bone fracture, WISP-1 is expressed in mesenchymal cells surrounding the site of injury. WISP-1 could prevent premature chondrocytic differentiation and promote growth and accumulation of mesenchymal cells at the fracture site. During the reparation stage, WISP-1 expression was limited to the osteoblasts lining the periosteum and the islands of woven bone within the provisional callus. This suggests that WISP-1 could play a role in the production of the bone matrix. By 3 weeks post fracture, the bones were reunited by hard callus and, at this stage, bone remodeling is taking place. No WISP-1 expression could be detected at 21 days post fracture indicating that WISP-1 is not likely implicated in the bone remodeling process. The Wnt signaling pathway is induced during bone repair and WISP-1 could constitute a critical element of the Wnt downstream genes involved in fracture healing (Hadjiargyrou et al., J Biol Chem, 277: 30177-30182 (2002).

Other members of the CCN family were found to have functions related to skeletogenesis and bone homeostasis. Cyr61 is expressed in chondrocytes of the developing limbs, ribs, vertebrae and craniofacial elements where it promotes chondrogenic differentiation (O'Brien and Lau, Cell Growth Differ, 3: 645-654 (1992); Wong et al. Dev Biol, 192: 492-508 (1997). During embryogenesis, CTGF expression is associated with condensed connective tissue and osteoblasts around bone and cartilage and promotes chondrocyte and osteoblast proliferation and differentiation and is involved in mineralization (Friedrichsen et al. Cell Tissue Res, 312: 175-88 (2003); Safadi et al., J Cell Physiol, 196: 51-62 (2003). NOV expression is found in chondrocytes, osteoclasts and osteoblasts and may play a role in sustaining the growth of osteoblast-like cells (Manara et al., Am J Pathol, 160: 849-859 (2002). WISP-2 expression is localized to osteoblasts and chondrocytes where it is thought to play a role in bone turnover (Kumar et al., J Biol Chem, 274: 17123-17131 (1999). WISP-3 mutations are responsible for progressive pseudorheumatoid dysplasia and its association with post-natal growth regulation and cartilage homeostasis has been proposed (Hurvitz et al., Nat Genet, 23: 94-8 (1999).

During bone development, the various CCN family members show either overlapping or exclusive expression patterns and reported activities for individual members are either similar or opposing. In addition, several types of receptors including integrins (Lau and Lam, Exp Cell Res, 248: 44-57 (1999); Grzeszkiewicz et al., J Biol Chem, 276: 21943-50 (2001); Leu et al., J Biol Chem, 278: 33801-33808 (2003), low density lipoprotein-related protein (Segarini et al., J Biol Chem, 276: 40659-40667 (2001) and Notch (Sakamoto et al., J Biol Chem, 277: 29399-29405 (2002) were reported for this family.

### Example

An assay was conducted to examine binding specificity of certain WISP-1 antibodies. Full length mouse WISP-1 (GenBank accession number NM_{—}018865)and full length human WISP-1 (GenBank accession number AF100779) were cloned into an expression vector encoding the human IgG₁ Fc region downstream of the WISP-1 sequence. The resulting recombinant fusion protein (WISP-1-Fc) was synthesized in a baculovirus expression system using Sf9 insect cells and purified to homogeneity from serum-free conditioned medium by affinity chromatography on a Protein A-Sepharose 4 Fast Flow (Amersham Pharmacia Biotech). Full length human WISP-1 was also expressed with an amino terminal hexa-histidine tag (WISP-1-His) in an E. coli strain. The cell lysate was subjected to chromatography on a Ni²⁺-NTA agarose column (Qiagen). WISP-1-His was eluted with a 0 to 500 mM imidazole gradient. Fractions containing the eluted WISP-1-His were then pooled and dialyzed. Human WISP-1 from a mammalian expression system was obtained by lysing NRK cells stably transfected with human WISP-1 (Arnold Levine; Princeton University, Princeton, N.J.) with SDS-PAGE sample buffer. A control cell lysate was generated with NRK cells stably transfected with an empty vector.

WISP-1 (50 ng) from various expression systems was electrophoresed on a SDS polyacrylamide gel and electro-transferred onto polyvinyldifluoride (PVDF) membranes and probed with different WISP-1 monoclonal antibodies.

WISP-1 antibodies 3D11.D7 (also referred to herein as “3D11”), 11C2.C10 (also referred to herein as “11C2”), 9C11.C7 (also referred to herein as “9C11”) and 5D4.F6 (also referred to herein as “5D4”) bound specifically to WISP-1 generated from baculovirus, bacterial and mammalian expression systems. These antibodies did not bind to the murine WISP-1 from baculovirus and did not recognize any protein from the control lysate. The WISP-1 antibodies 6F8, 3A7, 10H12, 3A11, 6E3, 3H10, 5G1, and 10B1 recognized both human and murine WISP-1 only when generated with the baculovirus expression system. These antibodies did not recognize human WISP-1 when produced in a bacterial or mammalian expression system. The antibody from clone 9C10 did not bind to any protein after Western blot.

These results suggest that WISP-1 antibodies 3D11, 11C2, 9C11 and 5D4 specifically recognize human WISP-1 and can be used for WISP-1 detection by Western blot.

### Example

An assay was conducted to identify the epitopes recognized by the WISP-1 antibodies 11C2, 9C11, 5D4 and 3D11.

Full length human WISP-1 (GenBank accession number AF100779) was cloned into a pIRESpuro2 expression vector (Clontech Laboratories, Palo Alto, Calif.) encoding 6 histidines downstream of the WISP-1 sequence. Deletion mutants were also generated by removing one, two or three domains of human WISP-1. The resulting contructs were also cloned into the pIRESpuro2 expression vector. The nomenclature used to identify the different WISP-1 constructs refer to the domains they contain. Domain 1 is the insulin-like growth factor binding protein domain (IFGBP), domain 2 is the von Willebrand factor C (VWFc) domain, domain 3 is the thrombospondin (TSP) domain, and the domain 4 is the C-terminal (CT) domain. The variable region resides between domain 2 and 3.

The sequences encoding these domains of WISP-1 are as follows:

Sequences of WISP-1 Constructs

| | |
|---|---|
| Domain 1: | |
| GAATTCACCATGAGGTGGTTCCTGCCCTGGACGCTG | (SEQ ID NO:3) |
| GCAGCAGTGACAGCAGCAGCCGCCAGCACCGTCCTG | |
| GCCACGGCCCTCTCTCCAGCCCCTACGAGCATGGAC | |
| TTTACTCCAGCTCCACTGGAGGACACCTCCTCACGC | |
| CCCCAATTCTGCAAGTGGCCATGTGAGTGCCCGCCA | |
| TCCCCACCCCGCTGCCCGCTGGGGGTCAGCCTCATC | |
| ACAGATGGCTGTGAGTGCTGTAAGATGTGCGCTCAG | |
| CAGCTTGGGGACAACTGCACGGAGGCTGCCATCTGT | |
| GACCCCCACCGGGGCCTCTACTGTGACTACAGCGGG | |
| GACCGCCCGAGGTACGCAATAGGAGTGTGTGCACAG | |
| GCGGCCGCACACCACCATCACCATCACCATCACTAA | |
| GTGAGGCCGCATAGATAACTGATCCAGTGTGCTGGA | |
| ATTAATTC | |
| Domain 2: | |
| GAATTCACCATGAGGTGGTTCCTGCCCTGGACGCTG | (SEQ ID NO:4) |
| GCAGCAGTGACAGCAGCAGCCGCCAGCACCGTCCTG | |
| GCCACTGCAGTGGTCGGTGTGGGCTGCGTCCTGGAT | |
| GGGGTGCGCTACAACAACGGCCAGTCCTTCCAGCCT | |
| AACTGCAAGTACAACTGCACGTGCATCGACGGCGCG | |
| GTGGGCTGCACACCACTGTGCCTCCGAGTGCGCCCC | |
| CCGCGTCTCTGGTGCCCCCACCCGCGGCGCGTGAGC | |
| ATACCTGGCCACTGCTGTGAGCAGTGGGTATGTGCG | |
| GCCGCACACCACCATCACCATCACCATCACTAAGTG | |
| AGGCCGCATAGATAAC | |
| Domain 3: | |
| GAATTCACCATGAGGTGGTTCCTGCCCTGGACGCTG | (SEQ ID NO:5) |
| GCAGCAGTGACAGCAGCAGCCGCCAGCACCGTCCTG | |
| GCCACTGCAGCATGGCACAGGAACTGCATAGCCTAC | |
| ACAAGCCCCTGGAGCCCTTGCTCCACCAGCTGCGGC | |
| CTGGGGGTCTCCACTCGGATCTCCAATGTTAACGCC | |
| CAGTGCTGGCCTGAGCAAGAGAGCCGCCTCTGCAAC | |
| TTGCGGCCATGCGATGTGGACATCCATACACTCATT | |
| AAGGCGGCCGCACACCACCATCACCATCACCATCAC | |
| TAAGTGAGGCCGCATAGATAACTGATCCAGTGT | |
| Domain 4: | |
| GAATTCACCATGAGGTGGTTCCTGCCCTGGACGCTG | (SEQ ID NO:6) |
| GCAGCAGTGACAGCAGCAGCCGCCAGCACCGTCCTG | |
| GCCACTGCAGGGAAGAAGTGTCTGGCTGTGTACCAG | |
| CCAGAGGCATCCATGAACTTCACACTTGCGGGCTGC | |
| ATCAGCACACGCTCCTATCAACCCAAGTACTGTGGA | |
| GTTTGCATGGACAATAGGTGCTGCATCCCCTACAAG | |
| TCTAAGACTATCGACGTGTCCTTCCAGTGTCCTGAT | |
| GGGCTTGGCTTCTCCCGCCAGGTCCTATGGATTAAT | |
| GCCTGCTTCTGTAACCTGAGCTGTAGGAATCCCAAT | |
| GACATCTTTGCTGACTTGGAATCCTACCCTGACTTC | |
| TCAGAAATTGCCAACGCGGCCGCACACCACCATCAC | |
| CATCACCATCACTAAGTGAGGCCGCATAGATAACTG | |
| ATCCAGTGTG | |
| Domain 1, 2: | |
| GAATTCACCATGAGGTGGTTCCTGCCCTGGACGCTG | (SEQ ID NO:7) |
| GCAGCAGTGACAGCAGCAGCCGCCAGCACCGTCCTG | |
| GCCACGGCCCTCTCTCCAGCCCCTACGACCATGGAC | |
| TTTACTCCAGCTCCACTGGAGGACACCTCCTCACGC | |
| CCCCAATTCTGCAAGTGGCCATGTGAGTGCCCGCCA | |
| TCCCCACCCCGCTGCCCGCTGGGGGTCAGCCTCATC | |
| ACAGATGGCTGTGAGTGCTGTAAGATGTGCGCTCAG | |
| CAGCTTGGGGACAACTGCACGGAGGCTGCCATCTGT | |
| GACCCCCACCGGGGCCTCTACTGTGACTACAGCGGG | |
| GACCGCCCGAGGTACGCAATAGGAGTGTGTGCACAG | |
| GTGGTCGGTGTGGGCTGCGTCCTGGATGGGGTGCGC | |
| TACAACAACGGCCAGTCCTTCCAGCCTAACTGCAAG | |
| TACAACTGCACGTGCATCGACGGCGCGGTGGGCTGC | |
| ACACCACTGTGCCTCCGAGTGCGCCCCCCGCGTCTC | |
| TGGTGCCCCCACCCGCGGCGCGTGAGCATACCTGGC | |
| CACTGCTGTGAGCAGTGGGTATGTGCGGCCGCACAC | |
| CACCATCACCATCACCATCACTAAGTGAGGCCGCAT | |
| AGATAAC | |
| Domain 1, 2, 3: | |
| GAATTCACCATGAGGTGGTTCCTGCCCTGGACGCTG | (SEQ ID NO:8) |
| GCAGCAGTGACAGCAGCAGCCGCCAGCACCGTCCTG | |
| GCCACGGCCCTCTCTCCAGCCCCTACGACCATGGAC | |
| TTTACTCCAGCTCCACTGGAGGACACCTCCTCACGC | |
| CCCCAATTCTGCAGTGGCCATGTGAGTGCCCGCCAT | |
| CCCCACCCCGCTGCCCGCTGGGGGTCAGCCTCATCA | |
| CAGATTGGCTGTGAGTGCTGTAAGATGTGCGCTCAG | |
| CAGCTTGGGGACAACTGCACGGAGGCTGCCATCTGT | |
| GACCCCCACCGGGGCCTCTACTGTGACTACAGCGGG | |
| GACCGCCCGAGGTACGCAATAGGAGTGTGTGCACAG | |
| GTGGTCGGTGTGGGCTGCGTCCTGGATGGGGTGCGC | |
| TACAACAACGGCCAGTCCTTCCAGCCTAACTGCAAG | |
| TACAACTGCACGTGCATCGACGGCGCGGTGGGCTGC | |
| ACACCACTGTGCCTCCGAGTGCGCCCCCCGCGTCTC | |
| TGGTGCCCCCACCCGCGGCGCGTGAGCATACCTGGC | |
| CACTGCTGTGAGCAGTGGGTATGTGAGGACGACGCC | |
| AAGAGGCCACGCAAGACCGCACCCCGTGACACAGGA | |
| GCCTTCGATGCTGTGGGTGAGGTGGAGGCATGGCAC | |
| AGGAACTGCATAGCCTACACAAGCCCCTGGAGCCCT | |
| TGCTCCACCAGCTGCGGCCTGGGGGTCTCCACTCGG | |
| ATCTCCAATGTTAACGCCCAGTGCTGGCCTGAGCAA | |
| GAGAGCCGCCTCTGCAACTTGCGGCCATGCGATGTG | |
| GACATCCATACACTCATTAAGGCgGCCGCACACCAC | |
| CATCACCATCACCATCACTAAGTGAGGCCGCATAGA | |
| TAACTGATCCAGTGTGCTGGA | |
| Domain 1, 2, 4: | |
| GAATTCACCATGAGGTGGTTCCTGCCCTGGACGCTG | (SEQ ID NO:9) |
| GCAGCAGTGACAGCAGCAGCCGCCAGCACCGTCCTG | |
| GCCACGGCCCTCTCTCCAGCCCCTACGACCATGGAC | |
| TTTACTCCAGCTCCACTGGAGGACACCTCCTCACGC | |
| CCCCAATTCTGCAAGTGGCCATGTGAGTGCCCGCCA | |
| TCCCCACCCCGCTGCCCGCTGGGGGTCAGCCTCATC | |
| ACAGATGGCTGTGAGTGCTGTAAGATGTGCGCTCAG | |
| CAGCTTGGGGACAACTGCACGGAGGCTGCCATCTGT | |
| GACCCCCACCGGGGCCTCTACTGTGACTACAGCGGG | |
| GACCGCCCGAGGTACGCAATAGGAGTGTGTGCACAG | |
| GTGGTCGGTGTGGGCTGCGTCCTGGATGGGGTGCGC | |
| TACAACAACGGCCAGTCCTTCCAGCCTAACTGCAAG | |
| TACAACTGCACGTGCATCGACGGCGCGGTGGGCTGC | |
| ACACCACTGTGCCTCCGAGTGCGCCCCCCGCGTCTC | |
| TGGTGCCCCCACCCGCGGCGCGTGAGCATACCTGGC | |
| CACTGCTGTGAGCAGTGGGTATGTCTGCAGGCAGGG | |
| AAGAAGTGTCTGGCTGTGTACCAGCCAGAGGCATCC | |
| ATGAACTTCACACTTGCGGGCTGCATCAGCACACGC | |
| TCCTATCAACCCAAGTACTGTGGAGTTTGCATGGAC | |
| AATAGGTGCTGCATCCCCTACAAGTCTAAGACTATC | |
| GACGTGTCCTTCCAGTGTCCTGATGGGCTTGGCTTC | |
| TCCCGCCAGGTCCTATGGATTAATGCCTGCTTCTGT | |
| AACCTGAGCTGTAGGAATCCCAATGACATCTTTGCT | |
| GACTTGGAATCCTACCCTGACTTCTCAGAAATTGCC | |
| AACGCGGCCGCACACCACCATCACCATCACCATCAC | |
| TAAGTGAGGCCGCATAGATAACTGATCCAGTGTGCT | |
| GGAATTAATTCGCTGTCTGCGAGGGCCAGCTGTTGG | |
| GGTGAGTACTCCCTCTCAAAAGCGGGCATGACTTCT | |
| GCGCTA | |
| Domain 1, 3, 4: | |
| GAATTCACCATGAGGTGGTTCCTGCCCTGGACGCTG | (SEQ ID NO:10) |
| GCAGCAGTGACAGCAGCAGCCGCCAGCACCGTCCTG | |
| GCCACGGCCCTCTCTCCAGCCCCTACGACCATGGAC | |
| TTTACTCCAGCTCCACTGGAGGACACCTCCTCACGC | |
| CCCCAATTCTGCAAGTGGCCATGTGAGTGCCCGCCA | |
| TCCCCACCCCGCTGCCCGCTGGGGGTCAGCCTCATC | |
| ACAGATGGCTGTGAGTGCTGTAAGATGTGCGCTCAG | |
| CAGCTTGGGGACAACTGCACGGAGGCTGCCATCTGT | |
| GACCCCCACCGGGGCCTCTACTGTGACTACAGCGGG | |
| GACCGCCCGAGGTACGCAATAGGAGTGTGTGCGCAT | |
| GCTGTGGGTGAGGTGGAGGCATGGCACAGGAACTGC | |
| ATAGCCTACACAAGCCCCTGGAGCCCTTGCTCCACC | |
| AGCTGCGGCCTGGGGGTCTCCACTCGGATCTCCAAT | |
| GTTAACGCCCAGTGCTGGCCTGAGCAAGAGAGCCGC | |
| CTCTGCAACTTGCGGCCATGCGATGTGGACATCCAT | |
| ACACTCATTAAGGCAGGGAAGAAGTGTCTGGCTGTG | |
| TACCAGCCAGAGGCATCCATGAACTTCACACTTGCG | |
| GGCTGCATCAGCACACGCTCCTATCAACCCAAGTAC | |
| TGTGGAGTTTGCATGGACAATAGGTGCTGCATCCCC | |
| TACAAGTCTAAGACTATCGACGTGTCCTTCCAGTGT | |
| CCTGATGGGCTTGGCTTCTCCCGCCAGGTCCTATGG | |
| ATTAATGCCTGCTTCTGTAACCTGAGCTGTAGGAAT | |
| CCCAATGACATCTTTGCTGACTTGGAATCCTACCCT | |
| GACTTCTC | |
| Domain 2, 3, 4: | |
| GAATTCACCATGAGGTGGTTCCTGCCCTGG | (SEQ ID NO:11) |
| ACGCTGGCAGCAGTGACAGCAGCAGCCGCCAGCACC | |
| GTCCTGGCCACTGCAGTGGTCGGTGTGGGCTGCGTC | |
| CTGGATGGGGTGCGCTACAACAACGGCCAGTCCTTC | |
| CAGCCTAACTGCAAGTACAACTGCACGTGCATCGAC | |
| GGCGCGGTGGGCTGCACACCACTGTGCCTCCGAGTG | |
| CGCCCCCCGCGTCTCTGGTGCCCCCACCCGCGGCGC | |
| GTGAGCATACCTGGCCACTGCTGTGAGCAGTGGGTA | |
| TGTGAGGACGACGCCAAGAGGCCACGCAAGACCGCA | |
| CCCCGTGACACAGGAGCCTTCGATGCTGTGGGTGAG | |
| GTGGAGGCATGGCACAGGAACTGCATAGCCTACACA | |
| AGCCCCTGGAGCCCTTGCTCCACCAGCTGCGGCCTG | |
| GGGGTCTCCACTCGGATCTCCAATGTTAACGCCCAG | |
| TGCTGGCCTGAGCAAGAGAGCCGCCTCTGCAACTTG | |
| CGGCCATGCGATGTGGACATCCATACACTCATTAAG | |
| GCAGGGAAGAAGTGTCTGGCTGTGTACCAGCCAGAG | |
| GCATCCATGAACTTCACACTTGCGGGCTGCATCAGC | |
| ACACGCTCCTATCAACCCAAGTACTGTGGAGTTTGC | |
| ATGGACAATAGGTGCTGCATCCCCTACAAGTCTAAG | |
| ACTATCGACGTGTCCTTCCAGTGTCCTGATGGGCTT | |
| GGCTTCTCCCGCCAGGTCCTATGGATTAATGCCTGC | |
| TTCTGTAACCTGAGCTGTAGGAATCCCAATGACATC | |
| TTTGCTGACTTGGAATCCTACCCTGACTTCTCAGAA | |
| ATTGCCAACGCGGCCGCACACCACCATCACCATCAC | |
| CATCACTAAGTGAGGCCGCATAGATAACTGATCCAG | |
| TGTGCTGGAATTAATTCGCTGTCTGCGA | |

Cells (HEK 293T) were transfected with the different constructs, and the culture media was collected after 48 hours. One milliliter of culture media was incubated with 20 μl of cobalt-agarose for 1 hour, centrifuged and washed. The adsorbed proteins were eluted by heating the pellet at 100° C. for 5 minutes in 20 μl of SDS-PAGE sample buffer. The samples were electrophoresed, electro-transferred onto PVDF and probed with the different WISP-1 antibodies.

Antibodies 11C2, 9C11 and 5D4 recognized only WISP-1 constructs containing the 19 first amino acids of the variable region located between domain 2 and 3. The WISP-1 antibody 3D11 recognized only WISP-1 constructs containing the domain 1 (amino acids 24 to 117).

These results indicate that the antibodies 11C2, 9C11 and 5D4 recognize specifically the variable region of WISP-1 whereas the antibody 3D11 recognizes specifically the domain 1 of WISP-1.

### Example

An assay was conducted to identify the epitope recognized by the WISP-1 antibody 9C10.F5 (also referred to herein as “9C10”).

Culture media from HEK 293T cells transfected with the various WISP-1 deletion constructs (as described above) was incubated with 1 μg of WISP-1 antibody 9C10 and 20 μl of protein A-agarose for 1 hour at room temperature. The immunocomplex was precipitated by centrifugation and eluted by heating the pellet at 100° C. for 5 minutes in 20 μl of SDS-PAGE sample buffer. The samples were electrophoresed, electro-transferred onto PVDF and probed with WISP-1 antibody 11C2.

The antibody 9C10 immunoprecipitated only constructs containing the domain 1 of WISP-1. These results demonstrate that the antibody 9C10 specifically recognizes the domain 1 of WISP-1 and can be used for immunoprecipitation.

### Example

WISP-1 antibody 9C10 (100 μl of 2 μg/ml in carbonate buffer, pH 9.6) was coated to Maxisorb plates overnight at 4° C. The plates were blocked with 200 μl of PBS/3% BSA for 1 hour. A standard curve was made of serial dilutions of WISP-1-Fc (100 μl in PBS/3% BSA) and incubated for 1 hour. After the incubation, the plates were washed with 100 μl PBS/0.05% Tween and WISP-1 antibodies (100 μl of 2 μg/ml) in PBS/3% BSA (biotinylated 11C2 or 55B) were incubated for 1 hour. For biotinylated 11C2, the plates were further incubated with 2 μg/ml HRP-conjugated streptavidin. For 55B, the plates are washed and incubated with HRP-conjugated donkey anti-rabbit IgG for 1 hour. At the end of the incubation, the wells were washed 6 times with 200 μl of PBS containing 0.05% Tween-20, and the signal was visualized using 100 μl of the horseradish peroxidase chromogenic substrate TMB (Kirkegaard & Perry Laboratories). The reaction was stopped with 100 μl of 1 M phosphoric acid, and the OD at 450 nm was measured. Non-specific binding was determined in parallel incubations by omitting microtiter well coating. No signal was generated when WISP-1-Fc or a WISP-1 antibody was omitted.

Using the antibody 9C10 for capture and the antibodies 11C2 and 55B for detection, an ELISA was conducted capable of detecting concentration of WISP-1 as low as 0.4 μg/ml. This ELISA may be useful for detecting WISP-1 protein in biological fluids such as serum.

### Example

Maxisorb plates were coated overnight at 4° C. with 50 μl/well of 10 μg/ml heparin (Sigma). The non specific binding sites were blocked with 200 μl of PBS/3% BSA for 1 hour. The plates were then incubated for 1 hour with 50 μl of 6 μg/ml hWISP-1-Fc in PBS/3% BSA in the presence of serial dilutions of WISP-1 antibodies. The plates were washed with PBS/0.05% Tween and further incubated 1 hour with 50 μl of 2 μg/ml HRP conjugated anti-human IgG-Fc in PBS/3% BSA. The plates were washed, and 100 μl of HRP substrate (TMB) was added. The color development was stopped with 100 μl of 1 M phosphoric acid and the OD at 450 nm was measured.

The WISP-1 antibodies 11C2, 5D4 and 9C11 inhibited WISP-1 binding to heparin with an IC₅₀ of 1.9, 2.5 and 3.7 μg/ml, respectively. The antibody 3D11 moderately reduced WISP-1 binding to heparin with a maximal inhibition of 62% at the highest concentration tested (40 μg/ml). The antibody 9C10 did not attenuate WISP-1 heparin binding, showing an inhibition curve similar to the irrelevant antibody control.

These results demonstrate that antibodies recognizing the variable region can inhibit WISP-1 binding to heparin. Because the two WISP-1 antibodies recognizing domain 1 have little or no effect on WISP-1 binding to heparin, it is presently believed that the domain 1 is less likely to participate in this interaction.

### Example

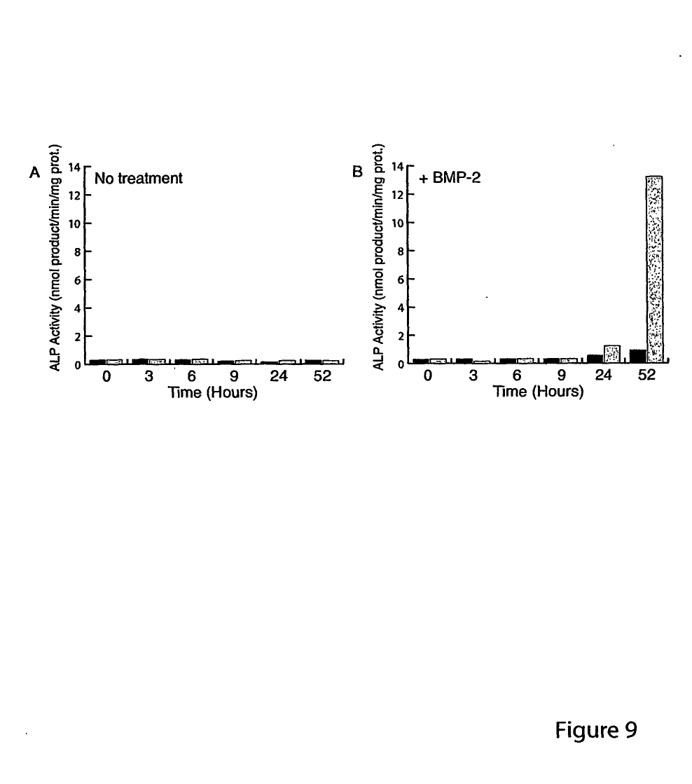

Because WISP-1 is induced during osteoblastic differentiation, its participation in this process was evaluated. C2C12 cells (ATCC) were transiently transfected with an empty vector (pIRES puro-2; BD Biosciences Clontech, Palo Alto, Calif.) (FIG. 9 black bars) or WISP-1 expression construct (WISP-1 cloned into pIRES puro-2; BD Biosciences Clontech, Palo Alto, Calif.) (FIG. 9 grey bars). Forty-eight hours after transfection, the culture media (DME/F12 medium supplemented with 15% FBS) was replaced by DME/F12 media containing 5% FBS (FIG. 9A) or DME/F12 media containing 5% FBS and 300 ng/ml BMP-2 (R & D Systems, Minneapolis, Minn.) (FIG. 9B). Alkaline phosphatase activity was measured at the indicated time using the following assay. Cells were washed twice with phosphate buffered saline (PBS) and lysed in 20 mM Tris, pH 7.4, 150 mM NaCl, 1% Triton x-100 for 5 minutes on ice. Twenty microliters of the lysate was added to 80 microliters of Attophos substrate (Roche) and incubated for 5 minutes at room temperature. The fluorescence was measured (excitation, 420 nm; emission, 560 nm) and the alkaline phosphatase activity was determined by comparison to a standard curve of enzymatic product. Cell lysates were analyzed for protein content using the micro-BCA assay kit (Pierce, Rockford, Ill.), and alkaline phosphatase activity was normalized for total protein concentration.

Although WISP-1 overexpression was not sufficient to trigger C2C12 cell osteoblastic differentiation (FIG. 9A), it greatly potentiated BMP-2 pro-osteoblastic activity (FIG. 9B). When treated with BMP-2, WISP-1 transfected cells demonstrated a 13-14 fold increase in alkaline phosphatase activity compared to cells transfected with a vector control. WISP-1 potentiation of pro-osteoblastic factors could promote lineage determination by facilitating the osteoblastic differentiation of progenitor cells.

### Example

A pSIREN-Shuttle vector (BD Biosciences Clontech, Palo Alto Calif.) expressing a small hairpin RNA (“shRNA”) construct specifically targeting WISP-1 was generated using the manufacturer's protocol (Protcol # PT3739-1). The following oligos were used to generate the WISP-1 targeting construct;

| | |
|---|---|
| forward: | |
| 5′-GATCCGATATGTGCCCAGCAGCTTTTCAAGAGA | (SEQ ID NO:12) |
| AAGCTGCTGGGCACATATCTTTTTTGCTAGCG-3′ | |
| and | |
| Reverse: | |
| 5′-AATTCGCTAGCAAAAAAGATATGTGCCCAGCAG | (SEQ ID NO:13) |
| CTTTCTCTTGAAAAGCTGCTGGGCACATATCG-3′. | |

C2C12 cells were transiently transfected with a vector expressing a control shRNA or a vector expressing a shRNA targeting WISP-1. Twenty-four hours after transfection, the culture media (described in the Example above) was replaced by media containing 5% FBS or media containing 5% FBS and 300 ng/ml BMP-2. WISP-1 expression and alkaline phosphatase activity were measured after 48 hours using the assay and materials described above.

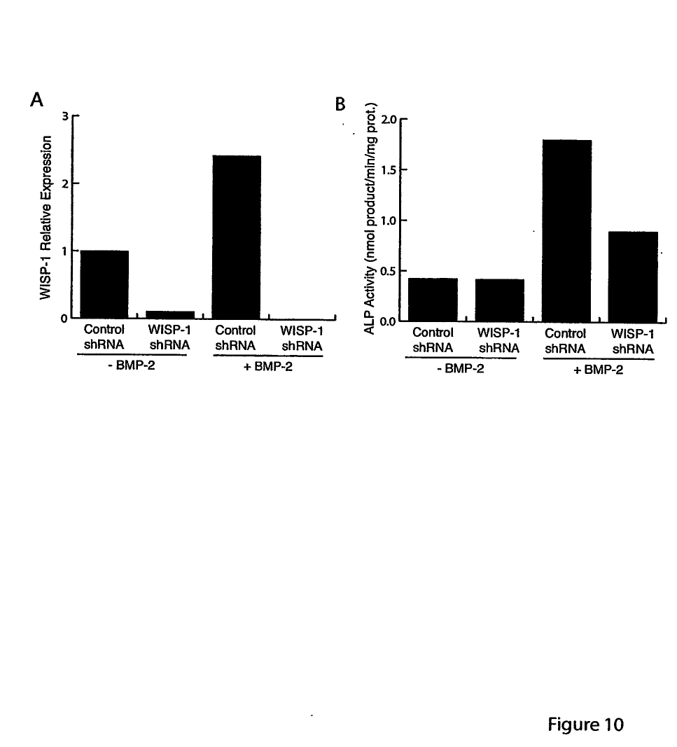

Compared to the shRNA control construct, the basal (−BMP-2) and BMP-2-induced WISP-1 expression (+BMP-2) were greatly reduced by the transfection of the WISP-1 targeting shRNA construct (FIG. 10A). Although WISP-1 knock-down was not sufficient to reduce basal alkaline phosphatase activity (−BMP-2), it significantly attenuated BMP-2-induced alkaline phosphatase activity (+BMP −2; FIG. 10B). The repression of BMP-2-induced osteoblastic differentiation by WISP-1 shRNA indicates that WISP-1 participates in osteogenesis by facilitating the osteoblastic differentiation of progenitor cells.

Deposit of Material

The following materials have been deposited with the American Type Culture Collection, 10801 University Blvd., Manassas, Va. 20110-2209, USA (ATCC):

| Material | ATCC Dep. No. | Deposit Date |
|---|---|---|
| 3D11.D7 | PTA-4624 | Sep. 4, 2002 |
| 11C2.C10 | PTA-4628 | Sep. 4, 2002 |
| 9C10.F5 | PTA-4626 | Sep. 4, 2002 |
| 5D4.F6 | PTA-4625 | Sep. 4, 2002 |
| 9C11.C7 | PTA-4627 | Sep. 4, 2002 |

This deposit was made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit. The deposit will be made available by ATCC under the terms of the Budapest Treaty, and subject to an agreement between Genentech, Inc. and ATCC, which assures permanent and unrestricted availability of the progeny of the culture of the deposit to the public upon issuance of the pertinent U.S. patent or upon laying open to the public of any U.S. or foreign patent application, whichever comes first, and assures availability of the progeny to one determined by the U.S. Commissioner of Patents and Trademarks to be entitled thereto according to 35 USC '122 and the Commissioner's rules pursuant thereto (including 37 CFR. '1.14 with particular reference to 886 OG 638).

The assignee of the present application has agreed that if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

The foregoing written description is considered to be sufficient to enable one skilled in the art to practice the invention. The present invention is not to be limited in scope by the example presented herein. Indeed, various modifications of the invention in addition to those shown and described herein will become apparent to those skilled in the art from the foregoing description and fall within the scope of the appended claims.

What is claimed is:

1. A method for treating damaged cartilage tissue comprising contacting said cartilage tissue with an effective amount of WISP antagonist.

2. The method of claim 1 wherein said WISP antagonist is selected from the group consisting of a WISP-1 antibody, WISP-1 immunoadhesin, WISP-1 polypeptide, and WISP-1 variant.

3. The method of claim 2 wherein said WISP-1 polypeptide consists of Domain 1 amino acids 24 to 117 of human WISP-1 (SEQ ID NO:1).

4. The method of claim 2 wherein said WISP antagonist is a WISP-1 monoclonal antibody.

5. The method of claim 4 wherein said WISP-1 monoclonal antibody is a human antibody, chimeric antibody or humanized antibody.

6. The method of claim 1 wherein said cartilage tissue is articular cartilage tissue.

7. The method of claim 1 wherein said effective amount of WISP antagonist is contacted with the damaged cartilage tissue in vivo in a mammal.

8. The method of claim 1 wherein said effective amount of WISP antagonist is contacted with the damaged cartilage tissue in vitro and subsequently transplanted into a mammal.

9. A method of stimulating differentiation of chondrocyte precursor cells, comprising contacting mammalian chondrocyte precursor cells with an effective amount of WISP antagonist.

10. The method of claim 9 wherein said WISP antagonist is selected from the group consisting of a WISP-1 antibody, WISP-1 immunoadhesin, WISP-1 polypeptide, and WISP-1 variant.

11. The method of claim 10 wherein said WISP-1 polypeptide consists of Domain 1 amino acids 24 to 117 of human WISP-1 (SEQ ID NO:1).

12. The method of claim 10 wherein said WISP antagonist is a WISP-1 monoclonal antibody.

13. The method of claim 12 wherein said WISP-1 monoclonal antibody is a human antibody, chimeric antibody or humanized antibody.

14. The method of claim 9 wherein said effective amount of WISP antagonist is contacted with the chondrocyte precursor cells in vivo in a mammal.

15. The method of claim 9 wherein said effective amount of WISP antagonist is contacted with the chondrocyte precursor cells in vitro and subsequently transplanted into a mammal.

16. A method of treating a cartilagenous disorder in a mammal, comprising administering an effective amount of WISP antagonist to said mammal.

17. The method of claim 16 wherein said WISP antagonist is selected from the group consisting of a WISP-1 antibody, WISP-1 immunoadhesin, WISP-1 polypeptide, and WISP-1 variant.

18. The method of claim 17 wherein said WISP-1 polypeptide consists of Domain 1 amino acids 24 to 117 of human WISP-1 (SEQ ID NO:1).

19. The method of claim 17 wherein said WISP antagonist is a WISP-1 monoclonal antibody.

20. The method of claim 19 wherein said WISP-1 monoclonal antibody is a human antibody, chimeric antibody or humanized antibody.

21. The method of claim 16 wherein said cartilagenous disorder is a degenerative cartilagenous disorder.

22. The method of claim 16 wherein said cartilagenous disorder is an articular cartilagenous diorder.

23. The method of claim 22 wherein said articular cartilagenous disorder is osteoarthritis or rheumatoid arthritis.

24. The method of claim 16 wherein said mammal is also treated using one or more surgical techniques.

25. The method of claim 24 wherein said effective amount of WISP antagonist is administered to the mammal prior to, after, and/or simultaneous with the surgical technique(s).

26. A kit or article of manufacture, comprising WISP antagonist and a carrier, excipient and/or stabilizer, and printed instructions for using said WISP antagonist to treat a cartilagenous disorder.
